(19) United States
(12) Patent Application Publication
(10) Pub. No.: US 2005/0287581 A1
(43) Pub. Date: Dec. 29, 2005

(54) Gene sequence of alpha(1,3)galactosyltransferase

(76) Inventors: John Clark, Midlothian (GB); Chris Denning, Midlothian (GB)

Correspondence Address: GERON CORPORATION, 230 CONSTITUTION DRIVE, MENLO PARK, CA, 94025, US

(21) Appl. No.: 11/198,685

(22) Filed: Aug. 4, 2005

Related U.S. Application Data
(62) Division of application No. 09/593,316, filed on Jun. 13, 2000.
(60) Provisional application No. 60/204,148, filed on May 15, 2000.

(51) Int. Cl. C12Q 1/68; C07H 21/04; C12N 9/10

(52) U.S. Cl. 435/6; 435/69.1; 435/193; 435/320.1; 435/325; 536/23.2

(57) ABSTRACT

Sequence data for the sheep α(1,3)galactosyltransferase (α1,3GT) gene is provided in this disclosure, which allows the user to construct vectors for expressing or inactivating the α1,3GT gene. This in turn enables the user to control expression of the Galα(1,3)Gal xenoantigen on the surface of cells.

### REFERENCE TO RELATED APPLICATIONS

This application is a divisional of U.S. Utility application Ser. No. 09/593,316, filed Jun. 13, 2000 (pending), through which it claims the priority benefit of U.S. Provisional Application 60/204,148, filed May 15, 2000. The priority applications are hereby incorporated herein by reference in its entirety.

### BACKGROUND

The acute shortage of human organs for transplantation is a compelling force towards the development of new sources of suitable tissue. One option that is gathering considerable attention is the use of organs from other animal species. The main challenge to overcome is rendering foreign tissue immunologically compatible with the patient being treated.

Tissue from most mammalian species would undergo hyperacute rejection when transplanted into humans. This is because human plasma contains natural antibodies against carbohydrate determinants of the animal tissue, thought to originate through prior immune stimulation by dietary antigen or mucosal microflora. Since the antibodies are pre-formed, rejection occurs within days of the transplant.

The main target for the natural antibodies mediating rejection is cell-surface oligosaccharides expressing the determinant Galα(1,3)Gal (reviewed by Joziasse et al., Biochim. Biophys. Acta 1455:403, 1999). Humans, apes and Old World monkeys differ from other mammals in that they lack α-galactosyl epitopes in complex oligosaccharides. Other mammals express the Galα(1,3)Gal epitope prominently on the surface of nucleated cells, including hepatic cells, renal cells, and vascular endothelium—which is especially problematic for xenotransplantation of whole organs.

The Galα(1,3)Gal epitope is made by a specific enzyme, α(1,3)galactosyltransferase, abbreviated in this disclosure as α1,3GT. The transferase uses UDP-galactose as a source of galactose, which it transfers specifically to an acceptor oligosaccharide, usually Galβ(1,4)GlcNAc (N-acetyl lactosamine). In mammals that don't express the Galα(1,3)Gal product, the α1,3GT locus is inactivated (Gailili et al., Proc. Natl. Acad. Sci. USA 15:7401, 1991). There are frameshift and nonsense mutations within the locus, turning it into a non-functional, processed pseudogene (Laarsen et al., J. Biol. Chem. 265:7055, 1990; Joziasse et al., J. Biol. Chem. 266:6991,1991).

In humans, N-acetyl lactosamine acceptor oligosaccharides are processed differently. The enzyme α(1,2)fucosyltransferase builds the N-acetyl lactosamine into Fucα(1,2)Galβ(1,4)GlcNAc, which is blood group H substance. This in turn serves as an acceptor substance for blood group A GlcNAc-transferase, or blood group B Gal-transferase, forming A-substance or B-substance, respectively, depending on the blood type of the individual. Naturally occurring antibodies circulating in the blood are reactive against the alternative carbohydrate determinants that are not self-antigens. Larsen et al. (Proc. Natl. Acad. Sci. USA 86:8227, 1989) isolated and characterized a cDNA encoding murine α1,3GT. Joziasse et al. (J. Biol. Chem. 267:5534, 1992) detected four distinct mRNA transcripts, which predict four different isoforms of the α1,3GT. The full-length mouse mRNA (including 5′ untranslated mRNA) was reported to span at least 35-kB of genomic DNA, distributed over nine exons ranging from 36 base pairs to ˜2600 base pairs in length. Numbering in the 5′ to 3′ direction, the coding region is distributed over Exons 4 to 9. The four transcripts are formed by alternative splicing of the pre-mRNA.

Joziasse et al. (J. Biol. Chem. 264:14290, 1989) isolated and characterized a cDNA encoding bovine cDNA. The coding sequence was predicted to be a membrane-bound protein with a large glycosylated COOH-terminal domain, a transmembrane domain, and a short NH2 terminal domain.

The porcine α1,3GT cDNA sequence has been reported from several different laboratories: Strahan et al. (Immunogenetics 41:101, 1995); U.S. Pat. No. 5,821,117; U.S. Pat. No. 5,849,991; and International Patent Application WO 95/28412. The genomic organization of porcine α1,3GT was reported by Katayama et al. (Glycoconjugate J. 15:83, 1998). Again, the coding region spans six exons, conserving the arrangement present in the mouse genome, and extending over nearly 24-kB.

It has been reported that about 95% of the naturally occurring xenospecific antibody in humans recognize the Galα(1,3)Gal epitope (McKensie et al., Transpl. Immunol. 2:81, 1994). Antibody in human serum binds specifically to pig endothelial cells in a manner that is inhibitable by Galα(1,3)Gal, or by Galα(1,6)Glu (melibiose). New age monkeys have the same naturally occurring antibody, and demonstrate hyperacute rejection of pig organ xenotransplants. The rejection reaction can be obviated in experimental animals by infusing the recipient with the free carbohydrate (Ye et al., Transplantation 58:330, 1994), or by adsorbing antibody from the circulation on a column of Galα(1,3)Gal or melibiose (Cooper et al., Xenotransplantation 3:102, 1996).

It has been suggested that xenotransplants of pig tissue could provide a source of various tissue components—heart valves, pancreatic islet cells, and perhaps large organs such as livers and kidneys (Cowley, Newsweek, Jan. 1, 2000). If xenotransplants from non-primates into humans is ever to become viable, then techniques need to be developed to suppress Galα(1,3)Gal expression. Possible genetic manipulation strategies are reviewed by Gustafsson et al. (Immunol. Rev. 141:59, 1994), Sandrin et al. (Frontiers Biosci. 2:e1-11, 1997), and Lavitrano et al. (Forum Genova 9:74, 1999).

One approach is to prevent the formation of Galα(1,3)Gal by providing another transferase that competes with α1,3GT for the N-acetyl lactosamine acceptor. International Patent Application WO 97/12035 (Nextran-Baxter) relates to transgenic animals that express at least one enzyme that masks or reduces the level of the xenoactive antigens by competing with α1,3GT. The enzymes proposed are α(1,2)fucosyltransferase (that makes H antigen in humans), α(2,6)sialyltransferase, and β(1,3)N-acetylglucosaminyltransferase. It is thought that once N-acetyl lactosamine has been converted by one of these transferases, it can no longer act as an acceptor for α1,3GT. The xenotransplantation cells of Application WO 97/12035 have at least one enzyme that reduces Galα(1,3)Gal expression, and also express a complement inhibitor such as CD59, decay accelerating factor (DAF), or membrane cofactor protein (MCP). Expression of human CD59 in transgenic pig organs enhances organ survival in an ex vivo xenogeneic perfusion model (Kroshus et al., Transplantation 61:1513, 1996).

Another approach is to disassemble Galα(1,3)Gal after it is formed. International Patent Application WO 95/33828 (Diacrin) suggests modifying cells for xenogeneic transplants by treating tissue with an α-glycosidase. Osman et al. (Proc. Natl. Acad. Sci. USA 23:4677, 1997) reported that combined transgenic expression of both α-glycosidase and α(1,2)fucosyltransferase leads to optimal reduction in Galα(1,3)Gal epitope. Splenocytes from mice overexpressing human α-glycosidase showed only a 15-25% reduction in binding of natural human anti-Galα(1,3)Gal antibodies. Mice overexpressing human α(1,2)fucosyltransferase as a transgene showed a reduction of Galα(1,3)Gal epitopes by ˜90%. Doubly transfected COS cells expressing both the glycosidase and the transferase showed negligible cell surface staining with anti-Galα(1,3)Gal, and were not susceptible to lysis by human serum containing antibody and complement.

A further alternative is to prevent Galα(1,3)Gal expression in the first place. Strahan et al. (Xenotransplantation 2:143, 1995) reported the use of antisense oligonucleotides for inhibiting pig α1,3GT, leading to a partial reduction in expression of the major target for human natural antibodies on pig vascular endothelial cells. Hayashi et al. (Transplant Proc. 29:2213, 1997) reported adenovirus-mediated gene transfer of antisense ribozyme for α1,3GT and αfucosyltransferase genes in xenotransplantation.

U.S. Pat. No. 5,849,991 (Bresatch) describes DNA constructs based on the mouse α1,3GT sequence. They are designed to disrupt expression of functional α1,3GT by undergoing homologous recombination across Exon 4, 7, 8, or 9. The constructs contain a selectable marker such as neoR, hygR or thymidine kinase. It is proposed that such constructs be introduced into mouse embryonic stem (ES) cells, and recovering cells in which at least one α1,3GT gene is inactivated. Experiments are reported which produced mice that are homozygous for inactivated α1,3GT, resulting in lack of expression of Galα(1,3)Gal epitope, as determined by specific antibody.

U.S. Pat. No. 5,821,117 (Austin Research Inst.) report cDNA sequence data for porcine α1,3GT. This was used to probe a pig genomic DNA library, and two lambda phage clones were obtained that contain different regions of the porcine transferase gene.

International Patent Application WO 95/28412 (Biotransplant) also report cDNA sequence data for porcine α1,3GT. It is proposed that genomic DNA fragments be isolated from an isogenic DNA library, and used to develop a gene-targeting cassette including a positive or negative selectable marker.

International Patent Application WO 99/21415 (Stem Cell Sciences, Biotransplant) reports construction of a DNA library from miniature swine. A vector is obtained comprising a pgk-neo cassette, and fragments of the α1,3GT gene. This is used for homologous recombination to eliminate α1,3GT activity in porcine embryonic fibroblasts.

Costa et al., Alexion Pharmaceuticals (Xenotransplantion 6:6, 1999) report experiments with transgenic mice expressing the human complement inhibitor CD59, with mice expressing α(1,2)fucosyltransferase, and with α1,3GT knock-outs. Expression of CD59 in combination of either α(1,2)fucosyltransferase or α1,3GT null phenotype prevented human serum-mediated cytolysis. Triple combination of all three modifications provided no additional protective effect.

There have been no reports of the use of α1,3GT inactivated tissue for xenotransplantation into humans. In view of the paucity of available organs for human transplantation, there is a pressing need to develop further options.

### SUMMARY OF THE INVENTION

Sequence data for the sheep α(1,3)galactosyltransferase (α1,3GT) gene is provided in this disclosure, which enables construction of vectors for expressing or inactivating the α1,3GT gene.

One embodiment of this invention is an isolated polynucleotide comprising a sequence of 30 or more consecutive nucleotides contained in (or capable of hybridization with) SEQ. ID NO:1 or 14 to 25, or which is contained in phage deposited in support of this application, but which does not appear in other known sequences, such as SEQ. ID NOs: 3, 5, 7, 9, 11, and 13. Included are polynucleotide constructs effective for expressing or inactivating a α(1,3)galactosyltransferase (α1,3GT) gene. Such polynucleotides can also be used in an assay for determining α1,3GT expression, in which the polynucleotide is combined with analyte mRNA or cDNA, and the formation of a duplex between the two is correlated with expression of α1,3GT by the cell.

Another embodiment of the invention is an isolated polypeptide comprising a sequence of 10 or more consecutive amino acids contained in or homologous to SEQ. ID NO:2, or encoded in a polynucleotide of this invention, which is not fully contained in any other known sequence, such as SEQ. ID NOs: 4, 6, 8, 10, and 12. Such polypeptides may have glycosyltransferase activity, or be immunologically cross-reactive with α1,3GT. In certain contexts, the polypeptides are used to prepare a Galα(1,3)Gal determinant by combining with a galactose acceptor saccharide in the presence of UDP-galactose.

Further embodiments of the invention are polyclonal and monoclonal antibodies (including immunoglobulin derivatives and equivalents) that bind specifically to a polypeptide of this invention, but not with other polypeptides. Some such antibodies react with α1,3GT of other mammalian species, while others are specific for ovine α1,3GT. They can be used, for example, in an assay to determine α1,3GT in a sample, in which the sample is combined with the antibody, and any complex formed is correlated with the presence of α1,3GT.

These and other embodiments of the invention will be apparent from the description that follows.

### BRIEF DESCRIPTION OF THE FIGURES

FIG. 1 is a chart providing certain sequence data of this invention, which is α(1,3)Galactosyltransferase cDNA in the sheep (α1,3GT) (SEQ. ID NO:1). The predicted protein sequence is shown below (SEQ. ID NO:2). Numbers 4 to 9 indicate the projected 5′ boundaries of Exons 4 to 9 in the genomic sequence.

FIG. 2 is a chart comparing the cDNA nucleotide sequence of sheep α1,3GT (SEQ. ID NO:1) with the bovine homolog (SEQ. ID NO:3). Identical residues are marked with an asterisk (*). The putative start codon is indicated with an arrow ( [symbol] ).

FIG. 3 is a chart comparing the protein sequence of sheep α1,3GT (SEQ. ID NO:2) with the bovine homolog (SEQ. ID NO:4). Identical residues are marked with an asterisk (*).

FIG. 4 is a half-tone reproduction, showing Southern blot analysis of sheep genomic DNA. Probes for two different α1,3GT exons yield one band, demonstrating that there is one copy of α1,3GT in the sheep genome.

FIG. 5 is a half-tone reproduction, showing Northern blot analysis of mRNA from sheep fetal fibroblasts. There is a single expressed transcription product for α1,3GT ˜3-kB in size.

FIG. 6 is a restriction map for the sheep α1,3GT gene. Phage clones were obtained for the regions shown, and used to determine intron sequences.

FIG. 7 is a map showing sequenced intron regions of α1,3GT. The areas numbered 1 to 9 in the figure correspond to SEQ. ID NOs:15, 16, 19, 20, 21, 22, 23, 24, and 25, respectively.

FIG. 8 is a map showing the design of two targeting vectors for inactivating the α1,3GT gene by eliminating one of the exons through homologous recombination. Each targeting vector comprises a selectable marker (neo), flanked on one side by an intron sequence of 1-2-kB, and on the other side by an intron sequence of 7-10-kB. A number of vectors have been obtained, including some that target Exon 4, Exon 8, and Exon9.

FIGS. 9 to 11 are drawings that compare the details of the targeting vectors p0054, p0079, and p0063, respectively, with the region of the α1,3GT gene sequence targeted for homologous recombination (shown below). These vectors are designed to replace the coding region in Exon 4 with the selectable marker neo or pac.

FIG. 12 is a drawing showing how vectors p0054 and p0079 can be truncated for use in an adeno-associated virus (AAV) vector.

FIG. 13 is a drawing that compares targeting vector p0078 (above), with the targeted Exon 8 region of α1,3GT (below), to be substituted with selectable marker neo.

FIGS. 14 and 15 are drawings that compare targeting vector p0047 and p0046, respectively, with the targeted Exon 9 region of α1,3GT (shown below), to be substituted with selectable marker neo.

FIG. 16 is a half-tone reproduction, showing PCR analysis of sheep fetal fibroblasts targeted with the p0054 vector. Using two primers for intron sequences that flank Exon 4 (upper panel), the expected product is 2.8-kB for native α1,3GT, and 2.2-kB after homologous recombination. Using a primer for the selectable marker (lower panel), amplification product is expected only after recombination. The results show that one of the samples is from a fibroblast that has successfully been targeted—replacing Exon 4 with the selectable marker. Since Exon 4 contains the translation initiation site, this would inactivate the α1,3GT gene.

FIG. 17 is a half-tone reproduction, showing PCR analysis of umbilical chord taken from a sheep fetus, generated by transfer of a α1,3GT targeted nucleus to a quiescent oocyte, which was implanted into a surrogate female at estrus. Two bands were observed: 2.8 kB, corresponding to the wild type α1,3GT gene, and 2.2 kB, appropriate for a targeted α1,3GT gene. These results are consistent with inactivation of the α1,3GT gene on one haplotype.

### DETAILED DESCRIPTION

This invention provides new sequence data for the α(1,3)galactosyltransferase (α1,3GT) gene in the Black Welsh Mountain sheep. The data includes the cDNA sequence, encompassing the entire α1,3GT encoding region, and certain portions of the genome sequence, from upstream of the coding region, from introns, and from downstream of the coding region.

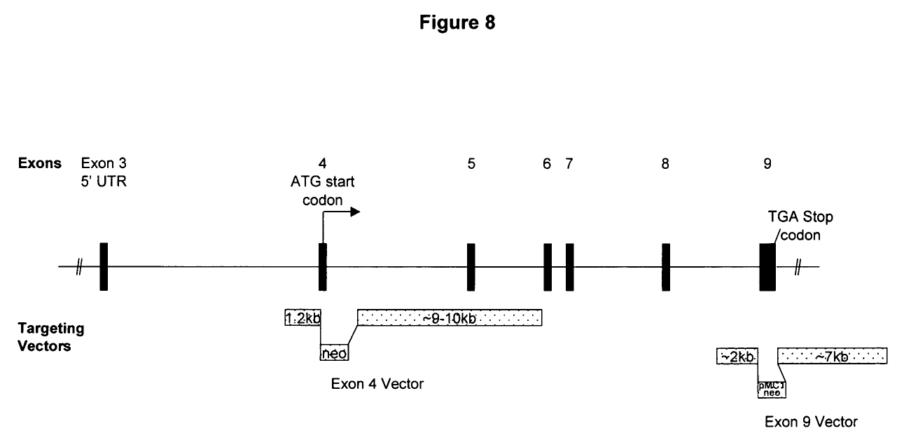

The discovery of this sequence and nucleic acids containing them have made it possible to construct targeting vectors designed to disrupt expression of a α1,3GT gene in eukaryotic cells, particularly sheep cells. Exemplary vectors targeting Exons 4 and 9 of the α1,3GT gene are shown in FIG. 8. These exemplary vectors have a knockout sequence flanked by two regions homologous to the genomic sequence. After recombination, the knockout sequence replaces a portion of the gene so as to disrupt transcription of a functioning α1,3GT—by elimination of Exon 4 or Exon 9, which encompass the translation start and stop codons, respectively. The knockout sequences in these exemplars comprise a selectable marker that facilitates the separation of cells that have been genetically altered after targeting with the vector.

For certain aspects of this invention, mammalian cells that have had an α1,3GT gene inactivated artificially in at least one haplotype are used to produce animals that do not express the Galα(1,3)Gal epitope. Nuclear transfer permits a nucleus having an inactivated α1,3GT gene to be transferred from a donor cell or cell line to an embryonic cell or oocyte. An embryo is then formed, engrafted into the uterus of a surrogate host, and used to birth a live neonate. Where the α1,3GT gene is homozygously inactivated, the animal or its progeny are already able to supply cells and tissue lacking the Galα(1,3)Gal epitope. Where the α1,3GT gene is inactivated in only one haplotype, homozygous inactivation can be achieved by standard cross-breeding techniques. Tissue lacking the α1,3GT epitope can then be used for transplantation into humans without risk of antigenicity for the natural antibody that is normally the linchpin for hyperacute rejection of xenografts.

### DEFINITIONS

For purposes of this disclosure, the term Galα(1,3)Gal (abbreviated GAL) refers to an oligosaccharide determinant present on endothelial cells and other cells of most non-primate mammals, for which humans have a naturally occurring antibody. The usual structure is Galα(1,3)Galβ(1,4)GlcNAc, although other forms of Galα(1,3)Gal specifically detectable by the naturally occurring anti Galα(1,3)Gal in human serum of B blood type are included. Galα(1,3)Gal is distinct from the Galα(1,3)[Fucα(1,2)]Galβ(1,4)GlcNAc determinant characteristic of the human B blood type antigen.

An “antibody detectable” determinant refers to a determinant that is present in an amount and is sufficiently accessible so that it can be detected by an antibody specific for the determinant in an appropriate immunoassay—such as an agglutination reaction, optionally developed with an antiglobulin reagent, or by immunohistochemistry.

The term “α(1,3)galactosyltransferase” and the abbreviation “α1,3GT” refer to the enzyme present in non-primate mammals that catalyzes the formation of the Galα(1,3)Gal determinant by attaching Gal in the α(1,3) position to the Galβ(1,4)GlcNAc acceptor. α1,3GT has the Enzyme Commission designation EC 2.4.1.124. α1,3GT is not naturally expressed in humans, and the term does not include the galactosyltransferase that forms the human B blood group antigen.

An “acceptor” substance for α1,3GT is a carbohydrate structure that can act as a substrate and become galactosylated as a result of transferase activity. Acceptors for α1,3GT include both Galβ(1,3)GlcNAc and Galβ(1,4)GlcNAc (Basu et al., J. Biol. Chem. 248:1700, 1973; Blake et al., J. Biol. Chem. 256:5387,1981).

A transferase is said to be “detectably expressed” by a cell at the mRNA level when mRNA encoding the transferase can be measured in the cell by some suitable technique, such as Northern analysis or PCR-reverse transcriptase. It may also be expressed at the protein level, as detected by a specific antibody or demonstration of the characteristic enzymatic activity. Those of skill in the art will recognize that some cells (such as mature red blood cells) do not express any glycosyl transferases, even though they display certain oligosaccharide determinants. Inhibition of α1,3GT expression is only meaningful in cells capable of expressing other glycosyl transferase enzymes. Most nucleated mammalian cells can be assumed to have this capability.

A gene is said to be “inactivated” when it is rendered incapable of transcribing a functional protein without further genetic engineering. For example, an inactivated gene may be missing necessary transcription or translation control elements, it may be lacking an essential part of the protein encoding region, or the encoding region may be placed out of phase. In a second example, the gene may be interrupted by an inserted sequence, or mutated in such a way as to interfere with transcription or translation of the gene product. In a third example, the inactivated gene may produce a translation product that has been altered in such a way that it lacks important enzymatic activity of the native gene product.

The term “tissue” refers to a heterogeneous collection of cells responsible for maintaining one or more physiological functions. Of interest for certain embodiments of this invention are organs suitable for transplantation, such as a whole kidney; however, the term also includes organ fragments and other embodiments, such as a piece of connective tissue, or a collection of cells in a medical support device.

The term “embryo” as it is used in this disclosure refers to an organism developing in the uterus of a species of interest at any time after fertilization or intrauterine transfer, not limited to a particular developmental period. The terms “engrafting” or “transplanting”, in reference to embryo manipulation, refer to any process known in the art for artificially introducing one or more embryos into the uterus of a female animal.

An “individual” or “subject” refers to any vertebrate animal, usually a member of a mammalian species, including but not limited to domestic animals, non-human primates, and humans.

It is understood that a clinical or biological “sample” encompasses a variety of sample types obtained from a subject and useful in an in vitro procedure, such as a diagnostic test. The definition encompasses solid tissue samples obtained during surgery or autopsy, and liquid samples such as blood, spinal fluid, and fluid obtained by amniocentesis, and various subtractions, enrichments, or solubilized extracts derived from such collections.

A “host cell” denotes a prokaryotic or eukaryotic cell has been genetically altered, or is capable of being genetically altered by administration of an exogenous polynucleotide, such as a bacterial plasmid or recombinant vector. When referring to genetically altered cells, the term refers both to the originally altered cell and to the progeny thereof.

A cell is said to be “genetically altered” when it contains an artificially introduced polynucleotide, or is the progeny of a cell so altered that has inherited an introduced polynucleotide. The polynucleotide may contain a sequence that is heterologous to the cell, it may contain native sequences in an artificial arrangement (e.g., an encoding region linked to a different promoter), or it may provide additional copies of a native encoding sequence. The polynucleotide can be introduced by transfection using electroporation or liposome-mediated transfer, homologous recombination, transduction using a viral vector, any combination thereof, or any other technique known in the art. The polynucleotide will often comprise a transcribable sequence encoding a protein of interest, which enables the cell to express the protein at an elevated level. In the context of the present application, a “genetically altered cell” has a genetic alteration that is inheritable by progeny of the cell. For example, an embryo having genetically altered DNA, if carried to term, will give rise to a neonate that has cells containing the genetic alteration.

The terms “polynucleotide” and “oligonucleotide” are used interchangeably to refer to a polymeric form of nucleotides of any length. Included are genes and gene fragments, mRNA, tRNA, rRNA, ribozymes, cDNA, recombinant polynucleotides, branched polynucleotides, plasmids, vectors, isolated DNA and RNA, nucleic acid probes, and primers. Also included are nucleotide analogs, including but not limited to thiol-derivatized nucleosides (U.S. Pat. No. 5,578,718), oligonucleotides with modified backbones (U.S. Pat. Nos. 5,541,307 and 5,378,825), and peptide nucleic acids (U.S. Pat. No. 5,786,461). The term polynucleotide, as used in this disclosure, refers interchangeably to double- and single-stranded molecules. Unless otherwise specified or required, any embodiment of the invention that is a polynucleotide encompasses both a double-stranded form, and each of the two complementary single-stranded forms known or predicted to make up the double-stranded form.

Polynucleotide sequences are said to be in a “non-natural arrangement” when they are joined together or interposed with another sequence in an arrangement not found in nature.

When comparison is made between polynucleotides for degree of identity, it is implicitly understood that complementary strands are easily generated, and the sense or antisense strand is selected or predicted that maximizes the degree of identity between the polynucleotides being compared. A computer program that can be used for finding homologous sequences is the BLAST algorithm (Example 1). For purposes of the present disclosure, percentage of sequence identity is calculated by first aligning the polynucleotide being examined with the reference counterpart, and then counting the number of residues shared between the sequences being compared as a percentage of the region under examination. No penalty is imposed for the presence of insertions or deletions, but insertions or deletions are permitted only where clearly required to readjust the alignment. The percentage is given in terms of residues in the sequence being examined that are identical to residues in the comparison or reference sequence. Particularly desirable polynucleotide sequences preserve the function of the prototype: depending on context, an ability to hybridize with a target sequence, the function of a polypeptide it may encode, or (for certain gene targeting vectors) the ability to facilitate homologous recombination or gene inactivation.

“Hybridization” refers to a reaction in which one or more polynucleotides react to form a complex that is stabilized via hydrogen bonding between the bases of the nucleotide residues. The hydrogen bonding can occur by Watson-Crick base pairing, Hoogsteen binding, triplex formation, or complexing in any other sequence-specific manner. A hybridization reaction will, on occasion, be a step in a more extensive process, such as part of PCR amplification. Hybridization reactions can be performed under conditions of different “stringency”. Conditions that increase the stringency of a hybridization reaction are widely known (see e.g., Sambrook et al., infra). Examples of conditions in order of increasing stringency: incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer, pH 7.2) and their equivalent using other buffer systems; formamide concentrations of 0%, 25%, 50%, and 75%; incubation times from 5 min to 24 h; 1, 2, or more washing steps; wash incubation times of 1, 5, or 15 min; and wash solutions of 6×SSC, 1×SSC, 0.1×SSC, or deionized water. Typical conditions of high stringency for the binding of a probe of about 100 base pairs and above is a hybridization reaction at 65° C. in 2×SSC, followed by repeat washes at 0.1×SSC—or the equivalent combination of solvent and temperature conditions for the particular nucleic acids being studied.

A “hybrid” of polynucleotides, or a “complex” formed between any two or more components in a biochemical reaction (such as antibody and antigen), refers to a duplex or higher-order complex that is sufficiently long-lasting to persist between its formation and subsequent detection.

A “control element” or “control sequence” is a nucleotide sequence involved in an interaction of molecules that contributes to the functional regulation of a polynucleotide, including replication, duplication, transcription, splicing, translation, or degradation of the polynucleotide.

“Operatively linked” refers to an operative relationship between genetic elements, in which the function of one element influences the function of another element. For example, an expressible encoding sequence may be operatively linked to control elements such as promoters and enhancers that permit transcription, and control elements for translation such as initiation sequences, stop codons, and signals for polyadenylation.

“Heterologous” means derived from a genotypically distinct entity from that of the rest of the entity to which it is being compared. For example, a polynucleotide introduced by genetic engineering techniques into an animal of a different species is said to be a heterologous polynucleotide. A promoter removed from its native coding sequence and operatively linked to a coding sequence with which it is not naturally found linked is said to be a heterologous promoter.

The terms “polypeptide”, “peptide” and “protein” are used interchangeably in this disclosure to refer to polymers of amino acids of any length. The polymer may comprise modified amino acids, it may be linear or branched, and it may be interrupted by non-amino acids. The terms also encompass an amino acid polymer that has been modified naturally or by intervention; for example, disulfide bond formation, glycosylation, lipidation, acetylation, and/or phosphorylation.

Percentage of sequence identity is calculated for polypeptides by first aligning the polypeptide being examined with the reference counterpart or prototype, and then counting the number of residues shared between the sequences being compared as a percentage of the region under examination. No penalty is imposed for the presence of insertions or deletions, but insertions or deletions are permitted only where clearly required to readjust the alignment. The percentage is given in terms of residues in the sequence being examined that are identical to residues in the comparison or reference sequence. Where substitutions are made, conservative substitutions (in which one amino acid is substituted by another with similar charge, size, hydrophobicity, or aromaticity) are typically better tolerated. Desirable sequences preserve the function of the prototype: for example, the enzymatic activity, the binding of specific substrates, and the binding of specific antibody as detectable in a standard competition inhibition immunoassay.

The term “antibody” as used in this disclosure refers to both polyclonal and monoclonal antibody. The ambit of the term deliberately encompasses not only intact immunoglobulin molecules, but also such fragments and genetically engineered derivatives of immunoglobulin molecules as may be prepared by techniques known in the art, and which retains the binding specificity of the antigen binding site.

An “immunogenic” compound or composition is capable of stimulating production of a specific immunological response when administered to a suitable host, usually a mammal.

An “isolated” polynucleotide, polypeptide, protein, antibody, or other substance refers to a preparation of the substance which is enriched in relation to some of the other components that may also be present in the environment where the substance or a similar substance is initially obtained or occurs naturally. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Enrichments by 2, 10, 100, and 1000 fold achieve improved degrees of purification. A substance can also be provided in an isolated state by a process of artificial assembly, such as by chemical synthesis or recombinant expression. An “isolated” cell is a cell that has been separated from the organism in which it was grown.

A polynucleotide used in a reaction, such as a probe used in a hybridization reaction or a vector used in gene targeting is referred to as “specific” or “selective” if it hybridizes or reacts with the intended target more frequently, more rapidly, or with greater duration than it does with alternative substances. Similarly, a polypeptide is referred to as “specific” or “selective” if it binds an intended target, such as a ligand, hapten, substrate, antibody, or other polypeptide more frequently, more rapidly, or with greater duration than it does to alternative substances. An antibody is referred to as “specific” or “selective” if it binds via at least one antigen recognition site to the intended target more frequently, more rapidly, or with greater duration than it does to alternative substances.

General Techniques

Unless otherwise noted, the practice of this invention can be carried out by employing standard techniques of genetic engineering, protein manipulation, and cell culture. Textbooks that describe standard laboratory techniques include “Molecular Cloning: A Laboratory Manual” 2nd Ed. (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Gene Transfer Vectors for Mammalian Cells” (J. M. Miller & M. P. Calos, eds., 1987); “Current Protocols in Molecular Biology” and “Short Protocols in Molecular Biology, 3rd Edition” (F. M. Ausubel et al., eds., 1987 & 1995); and “Recombinant DNA Methodology II” (R. Wu ed., Academic Press 1995). Techniques used in raising, purifying and modifying antibodies, and the design and execution of immunoassays, are described in Handbook of Experimental Immunology (D. M. Weir & C. C. Blackwell, eds.); The Immunoassay Handbook (Stockton Press NY, 1994); and R. Masseyeff, W. H. Albert, and N. A. Staines, eds., Methods of Immunological Analysis (Weinheim: VCH Verlags GmbH, 1993).

Texts that describe reproductive techniques and embryo transfer in animals include “Manual of the International Embryo Transfer Society: A procedural guide and general information for the use of embryo transfer technology emphasizing sanitary procedures”, 3^{rd} ed. (Stringfellow et al., Savoy, Ill.: International Embryo Transfer Society, Savoy Ill.); and “Embryo transfer in farm animals: A review of techniques and applications” (K. J. Betteridge, ed., Agriculture Canada Monographs No. 16, Ottawa, 1977).

Polynucleotides

The polynucleotides of this invention include those containing nucleotide sequences which are found within the α1,3GT cDNA sequence, shown in SEQ. ID NO:1, or the genomic sequence and its 5′ and 3′ flanking regions, part of which is shown in SEQ. ID NOs:15 to 25. Further sequence of the α1,3GT gene can be obtained by employing standard sequencing techniques known in the art to the phage plasmids deposited in support of this application.

Also included in this invention are polynucleotides containing α1,3GT-like sequence that is from naturally occurring allelic variants, synthetic variants, and homologs of α1,3GT with a percentage of residues identical to the α1,3GT cDNA or gene sequence, determined as described above. It is understood that substitutions, insertions, and deletions can be accommodated within a polynucleotide sequence without departing from the spirit of this invention. In certain embodiments, the polynucleotide sequences are at least 80% , 90%, 95%, 98%, or 100% identical to one of the sequences exemplified in this disclosure; in order of increasing preference. The length of consecutive residues in the identical or homologous sequence compared with the exemplary sequence can be at least about 15, 30, 50, 75, 100, 200 or 500 residues in order of increasing preference, up to the length of the entire clone, gene, or sequence.

Certain polynucleotides of this invention are distinct from polynucleotides already in the hands of the public, including previously known α1,3GT cDNA or genomic sequences from other species (e.g., SEQ. ID NO:3, 5, 7, 9 and 11), other glycosyl transferase cDNA and gene sequences, and matching fragments from other published sequence data that are fortuitously identical over the length of the fragment (for example, certain regions of SEQ. ID NO:13). A polynucleotide of this invention can be “distinct” from other polynucleotides because of an internal sequence difference (a substitution, deletion, or insertion), or because it is defined to encompass additional sequence at either end. Also included in the invention are recombinant or synthetic polynucleotides in which an α1,3GT-like sequence is linked to a heterologous sequence to form: for example, a heterologous promoter in an expression vector, or a selectable marker such as neo in a targeting vector.

Particular polynucleotides of this invention are useful for producing polypeptides of interest, as nucleotide probes and primers, and as targeting vectors for genetic knock-outs. Further description of the characteristics of such constructs is provided in a later section.

Preparation:

The polynucleotides of this invention can be prepared by any suitable technique in the art. Using the data provided in this disclosure or deduced from the deposited plasmids, sequences of less than ˜50 base pairs are conveniently prepared by chemical synthesis, either through a commercial service or by a known synthetic method, such as the triester method or the phosphite method. A suitable method is solid phase synthesis using mononucleoside phosphoramidite coupling units (Hirose et al., Tetra. Lett. 19:2449-2452, 1978; U.S. Pat. No. 4,415,732).

For use in antisense therapy, polynucleotides can be prepared by chemistry that produces compounds suitable stable for the pharmaceutical preparation for which they are intended. Non-limiting examples include thiol-derivatized nucleosides (U.S. Pat. No. 5,578,718), oligonucleotides with modified backbones (U.S. Pat. Nos. 5,541,307 and 5,378,825). Also of interest in the context of antisense constructs are peptide nucleic acids. Prototype PNAs have an achiral polyamide backbone consisting of N-(2-aminoethyl)glycine units, to which purine and pyrimidine bases are linked, for example, by way of a methylene carbonyl linker. PNAs are nuclease and protease resistant, and the uncharged nature of the PNA oligomers enhances the stability of PNA-nucleotide duplexes, thereby blocking transcription or translation. Uptake into cells can be enhanced by conjugating to lipophilic groups incorporating into liposomes, and introducing an amino acid side chain into the PNA backbone. See Soomets et al., Front. Biosci. 4:D782, 1999; U.S. Pat. Nos. 5,539,082, 5,766,855, 5,786,461, and International Patent Application WO 8/53801.

Polynucleotides of this invention can also be obtained by PCR amplification of a template with the desired sequence. Oligonucleotide primers spanning the desired sequence are annealed to the template, elongated by a DNA polymerase, and then melted at higher temperature so that the template and elongated oligonucleotides dissociate. The cycle is repeated until the desired amount of amplified polynucleotide is obtained (U.S. Pat. Nos. 4,683,195 and 4,683,202). Suitable templates depend on the nucleotide it is desired to obtain. Certain polynucleotides of this invention can be obtained from the deposited plasmids. Polynucleotides transcribed into mRNA can be obtained from the cDNA of a suitable cell in the desired species or a close relative. Cells that express α1,3GT include fibroblasts and endothelial cells. Intron sequences for α1,3GT can be obtained from a genomic DNA library of the species of interest.

Production scale amounts of large polynucleotides are most conveniently obtained by inserting the desired sequence into a suitable cloning vector and reproducing the clone. Techniques for nucleotide cloning are given in Sambrook, Fritsch & Maniatis (supra) and in U.S. Pat. No. 5,552,524. Exemplary cloning and expression methods are illustrated in Examples 1 and 2, below. Polynucleotides can be purified by standard techniques in nucleic acid chemistry, such as phenol-chloroform extraction, agarose gel electrophoresis, and other techniques known in the art, adapted according to the source of the polynucleotide.

Use of the Polynucleotides:

Polynucleotides of this invention can be used to identify α1,3GT nucleotide sequences in a sample of interest for research, diagnostic evaluation, or any other purpose. Generally, this will involve preparing a reaction mixture in which a sample suspected of containing an α1,3GT-related sequence is contacted with a polynucleotide of this invention under conditions that permit the polynucleotide to hybridize specifically with the compound being tested for, detecting any stable hybrids that form, and correlating the hybrids with the presence of a α1,3GT related sequence in the sample. The formation of stable hybrids can be detected by any suitable method known in the art. For example, the probe sequence with a detectable label such as a radioisotope, a chromophore, or a hapten such as avidin to which an signaling reagent can be attached. Alternatively, the reagent polynucleotide can be a primer for an amplification reaction in which the amount of product produced correlates with the formation of specific hybrids.

The specificity of the primer and stringency of hybridization conditions are both chosen with a view to facilitating detection of sequences of interest, while diminishing false positive reactions. Thus, when it is important to distinguish between α1,3GT sequences of different species, then stringency conditions should be high, and the reagent polynucleotide should be nearly identical to the sequence being tested for. Conditions can be determined empirically so that the reagent polynucleotide will hybridize with the α1,3GT sequence being tested for but not with α1,3GT sequences from other species. In a more usual application, the question to be addressed is whether sample taken from an animal of a particular species expresses autologous α1,3GT, or not.

For example, the polynucleotides of this invention can be used to determine whether a cell or tissue treated to inactivate the endogenous α1,3GT gene expresses α1,3GT at the mRNA level. Thus, polynucleotides of this invention are used as probes in mRNA blots or as primers in reverse PCR to detect endogenous α1,3GT species. False positives will not arise due to expression of α1,3GT from another species, but could arise from cross-reactivity from another glycosyl transferase sharing homology with α1,3GT, or with another expressed sequence with a fortuitous sequence match. Again, one of skill in the art will know how to select the reagent sequence and hybridization conditions based on sequence information and empirical testing. Since the degree of sequence identity with other galactosyl transferases is typically less than 60%, the practitioner has more latitude in modifying the reagent sequence to enhance stability, sensitivity, or for any other purpose.

Polynucleotides of this invention can also be used to inhibit the transcription or translation of α1,3GT in target cells, particularly those of the sheep. Such polynucleotides can be in the form of antisense constructs, which in some embodiments bind to α1,3GT mRNA and prevent translation. Other polynucleotides of this invention are ribozymes having a substrate (α1,3GT mRNA) binding portion, and an enzymatic portion with endonuclease activity that cleaves the substrate. The design and use of ribozymes is described in U.S. Pat. Nos. 4,987,071, 5,766,942, 5,998,193, and 6,025,167. Particular use of α1,3GT sequences for the inhibition of porcine α1,3GT synthesis is outlined in Strahan et al. (Xenotransplantation 2:143, 1995). The effectiveness of transcription inhibitors can be determined by transfecting a cell expressing α1,3GT with a test compound, and measuring any alteration of expression by the cell—either at the level of mRNA expression (e.g., by quantitative mRNA blotting or reverse PCR), at the level of protein expression (e.g., by immunoassay using anti-α1,3GT), or at the level of Galα(1,3)Gal expression (e.g., by binding of a specific antibody or a specific lectin).

Of particular interest are polynucleotide constructs comprising α1,3GT sequence of this invention that can be used for altering an endogenous α1,3GT gene. In general, such constructs have a region of at least about 200 base pairs and more typically at least about 1-kB that are at least about 95% identical to the genomic sequence of interest, to permit specific targeting. The construct will also incorporate a substitution, deletion, or insertion designed to modify the targeted gene.

Also included in this invention are polynucleotides that encode polypeptides of interest. Characteristics of the polypeptides of this invention are described in the section that follows. For polypeptides that are fragments of naturally occurring α1,3GT, there will be a corresponding naturally occurring polynucleotide encoding sequence. Those skilled in the art will recognize that because of redundancies in the amino acid code, any polynucleotide that encodes a peptide of interest can be used in a translation system to produce the peptide. Except where otherwise required, all possible codon combinations that translate into the peptide sequence of interest are included in the scope of the invention.

The polynucleotides of this invention can be in the form of an expression vector, in which the encoding sequence is operatively linked to control elements for transcription and translation in a prokaryotic or eukaryotic host cell of interest. Further details of expression systems for in vitro peptide production are provided below.

Also contemplated in this invention are constructs for introducing a transgene into a eukaryotic cell, for purposes of expressing α1,3GT, and potentially forming the Galα(1,3)Gal determinant on cells that would not otherwise present it. Suitable promoters include the endogenous α1,3GT promoter, predicted to be present in the α1,3GT genome sequence upstream from the translation start site. Other suitable promoters include constitutive promoters such as those for SV40 and CMV. J. H. White (Adv. Pharmacol. 40:339, 1997) describes modified steroid receptors and steroid-inducible promoters as genetic switches for gene therapy. Walther et al. (J Mol Med. 74:379, 1996) describe cell-type specific and inducible promoters for vectors in gene therapy as an approach for cell targeting.

In particular embodiments, the transgene is inserted into the genome of an embryonic cell (or a nucleus subsequently transferred to an embryonic cell). U.S. Pat. No. 4,873,191 describes a process in which genetic material is introduced into a zygote so that it is genetically transformed, then transplanting the embryo into a female so that the embryo develops to term. International Patent Application WO 99/58703 reports a method of preparing transgenic sheep by microinjecting an expression system into a fertilized oosperm, identifying embryonic cells using a nested PCR technique, and then transferring a multicellular embryo into a suitable host. U.S. Pat. No. 5,700,671 reports a method for producing mammals with a transgene that encodes a heterologous glycosyltransferase. Other techniques useful in preparing transgenic animals can be found, for example, in U.S. Pat. Nos. 4,736,666 5,741,957, and 5,942,435. Individuals with an expressible α1,3GT transgene, will not have naturally occurring antibodies against Galα(1,3)Gal, and should accommodate a xenotransplant from other animals expressing α1,3GT High α1,3GT activity in a transgenic cell will also decrease the availability of N-acetyl lactosamine acceptor carbohydrate for other glycosyltransferases using the same acceptor, shifting the balance of synthesized oligosaccharide structures away from the products of other transferases.

Polypeptides

The polypeptides of this invention include those that comprise amino acid sequences encoded within any of the polynucleotides of this invention, exemplified by SEQ. ID NO:2 and fragments thereof. Also included in this invention are polypeptides containing α1,3GT-like sequence that is from naturally occurring allelic variants, synthetic variants, and homologs of α1,3GT with a percentage of residues identical to the α1,3GT protein, calculated as described earlier.

It is understood that substitutions, insertions, and deletions can be accommodated within a protein sequence without departing from the spirit of this invention. Conservative substitutions are typically more tolerable, such as the substitution of charged amino acids with amino acids having the same charge, or substituting aromatic or lipophilic amino acids with others having similar features. Certain peptides of this invention are at least 60%, 80%, 90%, 95%, or 100% identical to one of the sequences exemplified in this disclosure; in order if increasing preference. The length of the identical or homologous sequence compared with the prototype polypeptide can be about 7, 10, 15, 20, 30, 50 or 100 residues in order of increasing preference, up to the length of the entire protein.

Certain peptides of this invention are distinct from peptides already in the hands of the public, including previously known α1,3GT proteins and non-functional homologs from other species (e.g., SEQ. ID NOs:4, 6, 8, 10, and 12), and other glycosyl transferases, such as the human A and B blood group transferases. A polypeptide of this invention can be “distinct” from other polypeptides because of an internal sequence difference (a substitution, deletion, or insertion), or because it is defined to encompass additional sequence at either end. Also included in the invention are artificially engineered fusion proteins in which a α1,3GT-like sequence is linked to a heterologous sequence which modulates α1,3GT activity, provides a complementary function, acts as a tag for purposes of labeling or affinity purification, or has any other desirable purpose.

Particular peptides of this invention are useful for their galactose transferase activity, for drug screening, and for raising specific antibody, as described below. mRNA splice variants of mouse α1,3GT have been observed that omit Exons 5 and 6, and may still produce functional enzyme. If the ovine α1,3GT is analogous, then the catalytic activity probably resides somewhere else in the molecule. It is predicted that the catalytic activity of α1,3GT resides further towards the —COOH terminus, probably at least partly in Exon 9.

Preparation:

Short polypeptides of this invention can be prepared by solid-phase chemical synthesis. The principles of solid phase chemical synthesis can be found in Dugas & Penney, Bioorganic Chemistry, Springer-Verlag NY pp 54-92 (1981), and U.S. Pat. No. 4,493,795. Automated solid-phase peptide synthesis can be performed using devices such as a PE-Applied Biosystems 430A peptide synthesizer (commercially available from Applied Biosystems, Foster City Calif.).

Longer polypeptides are conveniently obtained by translation in an in vitro translation system, or by expression in a suitable host cell. To produce an expression vector, a polynucleotide encoding the desired polypeptide is operably linked to control elements for transcription and translation, and then transfected into a suitable host cell. Expression may be effected in prokaryotes such as E. coli (ATCC Accession No. 31446 or 27325), eukaryotic microorganisms such as the yeast Saccharomyces cerevisiae, or higher eukaryotes, such as insect or mammalian cells. Control elements such as the promoter are chosen to permit translation at an acceptable rate under desired conditions. A number of expression systems suitable for producing the peptides of this invention are described in U.S. Pat. No. 5,552,524. Expression cloning is available from such commercial services as Lark Technologies, Houston Tex.

Following production, the protein is typically purified from the producing host cell by standard methods in protein chemistry in an appropriate combination, which may include ion exchange chromatography, affinity chromatography, and HPLC. Expression products are optionally produced with a sequence tag to facilitate affinity purification, which can subsequently be removed by proteolytic cleavage.

Use of the Polypeptides:

Polypeptides of this invention can be used for a number of worthwhile purposes, including but not limited to the production of Galα(1,3)Gal-containing synthetic oligosaccharides, for drug screening, and for production of antibody.

At least three activities are present in the native α1,3GT: binding of the substrate UDP-gal, binding of the acceptor oligosaccharide N-acetyl lactosamine, and catalytic activity whereby the acceptor oligosaccharide is galactosylated. The binding site for each acceptor can be determined by contacting a test fragment or homolog with the substrate, or with a competitive inhibitor that would be expected to bind with higher affinity, and determining whether a complex forms by a suitable separation technique, such as HPLC, or by an equilibrium quantitation technique, such as microchip analysis, which will better detect low-affinity reactions.

The site of catalytic activity can be determined by contacting the test peptide with both UDP-galactose and acceptor oligosaccharide, and measuring any Galα(1,3)Gal formed, for example, by immunoassay. Optionally, a systematic approach can be used to determine functional regions and homologs of α1,3GT according to any of these assays. For example, the assay system is confirmed on the native α1,3GT structure; then a series of nested fragments is tested to determine the minimum fragment that provides the same activity. Similarly, substitutions can be introduced into the sequence until the activity is ablated, thereby determining what residues are critical. Henion et al. (Glycobiology 4:193, 1994) characterized the minimal size for catalytically active α1,3GT in New World monkeys, finding that ˜68 amino acids could be removed from the N-terminal end before losing activity, while removal of only ˜3 amino acids from the C-terminal end resulted in loss of activity.

Native α1,3GT, and fragments and homologs that preserve substrate binding or catalytic activity, can be used to screen for activity inhibitors and enhancers. For example, a reaction mixture can be prepared containing UDP-galactose, a carbohydrate acceptor, a peptide having α1,3GT activity, and a test compound. The rate of formation of Galα(1,3)Gal is then measured. When compared to a reaction mixture not containing the test compound, a decrease in activity correlates with inhibitory capacity of the test compound, while an increase in activity correlates with augmentation capacity.

Native α1,3GT, and fragments and homologs with α1,3GT activity are also of interest for the artificial production of complex oligosaccharides containing a Galα(1,3)Gal determinant, or which build upon the Galα(1,3)Gal linkage. Such structures may be employed for any worthwhile purpose. For example, oligosaccharides that terminate in Galα(1,3)Gal are of interest for use in the context of minimizing a rejection reaction in an individual xenotransplanted with a Galα(1,3)Gal-expressing antigen. Substances containing a high density of the determinant can be used as an adsorbant to remove naturally occurring anti-Galα(1,3)Gal antibodies from plasma by ex vivo recirculation, or by adsorption in situ (see WO 98/42750). Certain Galα(1,3)Gal polymers, built into structures that have T-cell inhibitory epitopes, are proposed as immunosuppressants for the natural antibody against Galα(1,3)Gal. Catalytic α1,3GT peptides can optionally be adapted with amino acid additions, deletions, or substitutions for any worthwhile purpose, such as to improve stability or modify the specificity of the transferase in any way that is desirable.

Peptides of this invention are also of interest for the preparation and testing of antibodies against α1,3GT. A polyclonal antiserum raised against intact α1,3GT can be screened to map immunogenic portions of the primary sequence. To do this, sequential peptides about 12 residues long are synthesized that cover the entire protein (SEQ. ID NO:2), and overlapping by about 8 residues. The peptides can be prepared on a nylon membrane support by standard F-Moc chemistry, using a SPOTS™ kit from Genosys according to manufacturer's directions. Prepared membranes are overlaid with the antiserum, washed, and overlaid with β-galactosidase conjugated anti-immunoglobulin. Positive staining identifies antigenic regions, which, in an appropriate context, may themselves be immunogenic. There will also be antibodies that span different parts of the primary structure, or which rely on a conformational component not displayed in smaller peptides.

Suitable methods for raising and testing α1,3GT antibodies are described in the following section.

Antibodies

Antibody molecules of this invention include those that are specific for any novel peptide encompassed in this disclosure. These antibodies are useful for a number of purposes, including assaying for the expression of α1,3GT, and purification of α1,3GT peptides by affinity purification.

Polyclonal antibodies can be prepared by injecting a vertebrate with a polypeptide of this invention in an immunogenic form. If needed, immunogenicity of a polypeptide can be enhanced by linking to a carrier such as KLH, or combining with an adjuvant, such as Freund's adjuvant. Typically, a priming injection is followed by a booster injection is after about 4 weeks, and antiserum is harvested a week later. If desired, the specific antibody activity can be further purified by a combination of techniques, which may include Protein-A chromatography, ammonium sulfate precipitation, ion exchange chromatography, HPLC, and immunoaffinity chromatography using the immunizing polypeptide coupled to a solid support. Antibody fragments and other derivatives can be prepared by standard immunochemical methods, such as subjecting the antibody to cleavage with enzymes such as papain, pepsin, or trypsin.

Any unwanted cross-reactivity can be removed by treating the polyclonal antibody mixture with adsorbants made of those antigens attached to a solid phase, and collecting the unbound fraction. Contaminating activity against other transferases of the same species, or against α1,3GT from other species, can all be removed by this technique if such cross-reactivity would interfere with the intended use of the antibody. Specificity of the original antisera can be improved to start with, by immunizing with peptide fragments of α1,3GT that are substantially distinct from the equivalent region of the homologous protein.

Production of monoclonal antibodies is described in such standard references as Harrow & Lane (1988), U.S. Pat. Nos. 4,491,632, 4,472,500 and 4,444,887, and Methods in Enzymology 73B:3 (1981). Briefly, a mammal is immunized as described above, and antibody-producing cells (usually splenocytes) are harvested. Cells are immortalized, for example, by fusion with a non-producing myeloma, transfecting with Epstein Barr Virus, or transforming with oncogenic DNA. The treated cells are cloned and cultured, and the clones are selected that produce antibody of the desired specificity.

Other methods of obtaining specific antibody molecules (optimally in the form of single-chain variable regions) involve contacting a library of immunocompetent cells or viral particles with the target antigen, and growing out positively selected clones. Immunocompetent phage can be constructed to express immunoglobulin variable region segments on their surface. See Marks et al., New Eng. J. Med. 335:730, 1996, International Patent Applications WO 94/13804, WO 92/01047, WO 90/02809, and McGuiness et al., Nature Biotechnol. 14:1449, 1996.

The antibodies of this invention are can be used in immunoassays to detect or quantitate any of the polypeptides of this invention, including α1,3GT. For example, it may be desirable to measure α1,3GT in a biological sample to determine whether an individual expresses α1,3GT (and hence the Galα1,3Gal epitope), or whether a cell has been successfully treated to inactivate the α1,3GT gene. It may also be desirable to measure α1,3GT in a biological sample taken from an individual suspected of having a disease that is correlated with altered expression of α1,3GT,

General techniques of immunoassay can be found in “The Immunoassay Handbook”, Stockton Press NY, 1994; and “Methods of Immunological Analysis”, Weinheim: VCH Verlags gesellschaft mbH, 1993). The antibody is combined with a test sample under conditions where the antibody will bind specifically to any modulator that might be present, but not any other proteins liable to be in the sample. The complex formed can be measured in situ (U.S. Pat. Nos. 4,208,479 and 4,708,929), or by physically separating it from unreacted reagents (U.S. Pat. No. 3,646,346). Separation assays typically involve labeled α1,3GT reagent (competition assay), or labeled antibody (sandwich assay) to facilitate detection and quantitation of the complex. Suitable labels are radioisotopes such as ¹²⁵I, enzymes such as β-galactosidase, and fluorescent labels such as fluorescein. Antibodies of this invention can also be used to detect α1,3GT in tissue sections by such techniques as immunohistology or flow cytometry. The antibody is contacted with the tissue, unreacted antibody is washed away, and then bound antibody is detected—either directly, or by using a labeled anti-immunoglobulin reagent.

Assays of this nature can also be used in a competitive format to identify antibodies that bind to the same epitope on a target compound. In one such format, the reference antibody is labeled, and tested for binding to α1,3GT or a related peptide under conditions that permit specific antibody-peptide complexes to form. In parallel, the α1,3GT peptide is first reacted with the test antibody, and the labeled reference antibody is added subsequently. The assay is read by separating free antibody from peptide-antibody complexes (e.g., by gel filtration or precipitation), and then determining the proportion of label associated with the complexes. If the bound proportion decreases as a result of preincubating with the test antibody, then the test antibody and the reference antibody share overlapping or conformationally related epitopes. Epitope sharing can also be evaluated by mapping the binding site of each antibody using peptide fragments, described earlier. Antibodies can also be screened to identify those with catalytic inhibitory capacity, according to the inhibition screening assay described in the preceding section

Inactivation of the α(1,3)Galactosyltransferase Gene

This invention provides cells and cell nuclei, in which a preexisting gene for α1,3GT has been inactivated. This means that synthesis of the Galα(1,3)Gal epitope by at least one α1,3GT allele has been suppressed.

A gene for α1,3GT can be inactivated by any one of a number of transformations. For example, the gene may be adapted so that no mRNA transcript is produced; for example, by deleting or altering a transcription control element, such as a promoter or a transcription start sequence. Alternatively, the gene may be adapted so that any mRNA that is produced is not transcribable into the protein product. This can be effected, for example, by deleting or altering a translation control element, such as a ribosomal binding site or a translation initiation codon. Alternatively, the gene may be adapted so that any protein that is produced lacks the essential features of a glycosyl transferase. For example, the encoding region can be interrupted with stop codons, the encoding region can be placed out-of-phase, or critical portions of the protein may be missing, such as a structural component or a signal peptide for secretion. In a related alternative, the gene may be adapted so that the protein product lacks the specificity of α1,3GT—either because the catalytic site is removed, or because substrate binding specificity has been sufficiently altered so that the enzyme is incapable of synthesizing the Galα(1,3)Gal linkage.

A principle method for inactivating the α1,3GT gene is to actually change the genetic structure, making the inactivation inheritable by progeny of the cell. Changes of this nature can be effected by disrupting the genome with an integrating vector or mutating the genome by chemical or biological treatment. Treated cells are then cloned, and tested to determine if α1,3GT expression has been affected. Suitable means of testing include determination of mRNA expression by probe or amplification based assays, or determination of α1,3GT protein expression by immunoassay of cell lysates or immunohistochemical staining of fixed cells.

Efficient targeting of the α1,3GT gene generally requires a targeting vector, comprising nucleotide sequence identical or nearly identical to a portion of the gene of interest, linked to another structure capable of introducing the alteration. One such method uses homologous recombination, in which a DNA vector comprising homologous regions recombines at the targeted site, substituting its DNA sequence for that of the target. Cloned cells that have been selectively targeted can be identified by PCR amplification of a sequence exclusive to the targeting vector, restriction analysis of the recombination site, or expression phenotype.

Generally it is more convenient to include a selectable marker in the targeting construct, so that targeted cells can rapidly be separated from untargeted cells. U.S. Pat. No. 5,614,396 describes a method for obtaining a cell containing a desired sequence in the cell's genome, by using a targeting vector having two regions homologous to the targeting sequence, flanking the sequence which is to be inserted, and also having a selectable marker. The DNA undergoes homologous recombination at the target site, and recombined cells are recovered under selective culture conditions. Positive selection markers include the neo gene, selectable using G418 or kanamycin; the hyg gene, selectable using hygromycin the gpt gene, selectable using xanthine, and hypoxanthine-phosphoribosyltransferase (HPRT), selectable using hypoxanthine. Negative selection markers include thymidine kinase(TK), selectable using acyclovir or ganciclovir; and HPRT, selectable using 6-thioguanine; and cytosine deaminase, selectable using 5-fluoro-cytosine. Markers can also be labels like green fluorescent protein or β-galactosidase, which permit clones of targeted cells to be identified and selected. In certain contexts, it is advantageous to use markers with reduced potential for antigenicity in humans. For example, U.S. Pat. No. 6,020,192 illustrates a humanized green fluorescent protein.

For effecting homologous recombination, U.S. Pat. Nos. 5,464,764 and 5,631,153 describe a double-selection strategy, in which two sequences homologous to the gene target flank a positive selection marker, and a negative selection marker is attached to the 3′ terminal of the second flanking region. Homologous integration retains the positive selection marker, but eliminates the negative selection marker, whereas random integration usually retains both markers. Thus, by screening for both markers sequentially or together, cells that have been correctly targeted will be positively selected, and those that have been incorrectly targeted are selected out. U.S. Pat. No. 5,789,215 reports the use of homologous recombination targeting vectors for modifying the cell genome of mouse embryonic stem cells. Other information of interest for homologous recombination targeting can be found in U.S. Pat. Nos. 5,589,369 and 5,776,774.

Example 3 describes illustrative targeting vectors according to this invention. They are capable of inactivating the sheep α1,3GT gene by eliminating part of the protein coding region via homologous recombination. The vectors comprise flanking regions identical to the targeted α1,3GT sequence, one side being about 1-kB, the other being at least 1 or 2-kB, in either order. In between the flanking regions is a selectable marker such as neo, designed to replace one of the Exons in the α1,3GT coding sequence. The selectable marker genes are not provided with their own promoter, and require continued translation through the upstream α1,3GT sequence in order to be expressed. This helps the marker select for properly integrated vector, because vector inserted at a random site will probably not link the marker gene to a suitable promoter, and resistance to the selector drug will not be conferred. This strategy is particularly effective for cell types that normally express a high level of α1,3GT and the Galα(1,3)Gal epitope. In these cells, the α1,3GT promoter will be highly active and the drug resistance marker will be strongly expressed. Thus, a higher concentration of selector drug can be used to select out cells that have the vector inserted elsewhere.

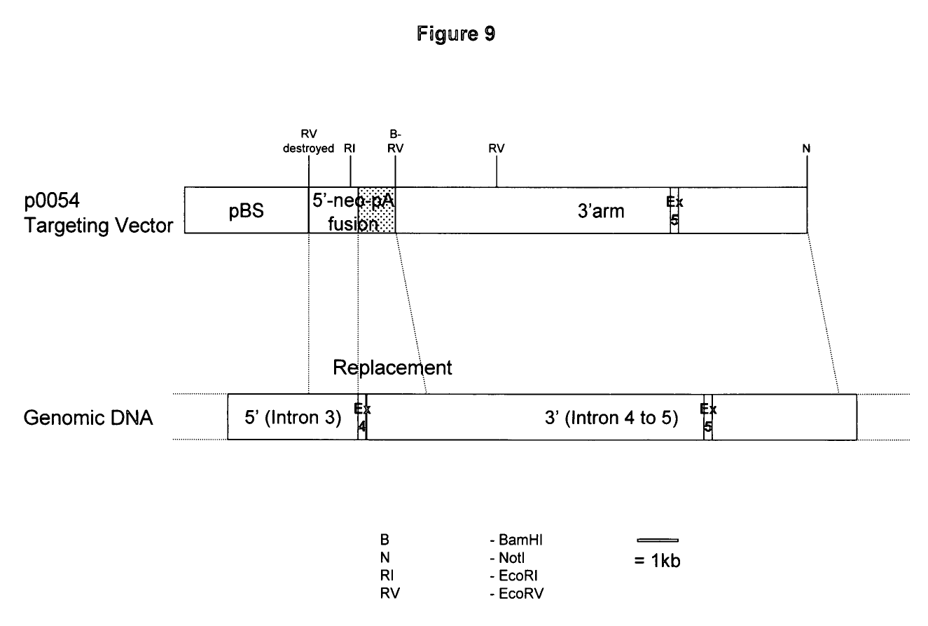

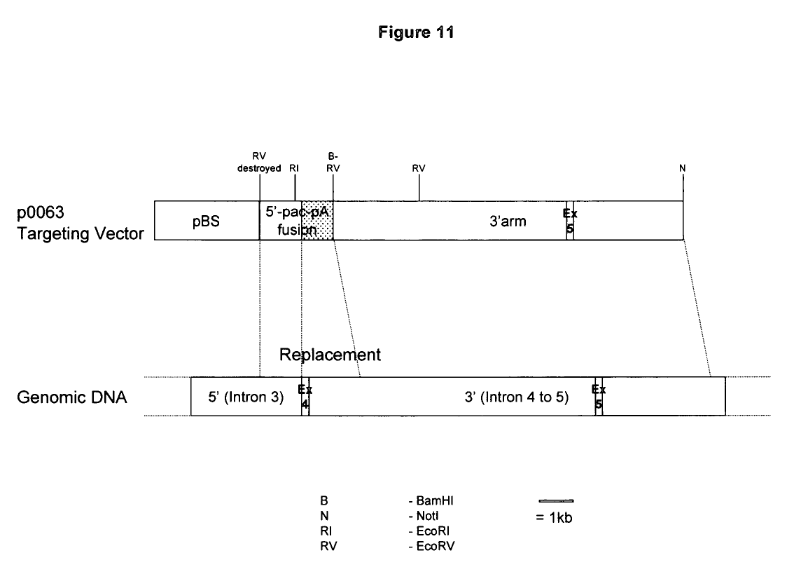

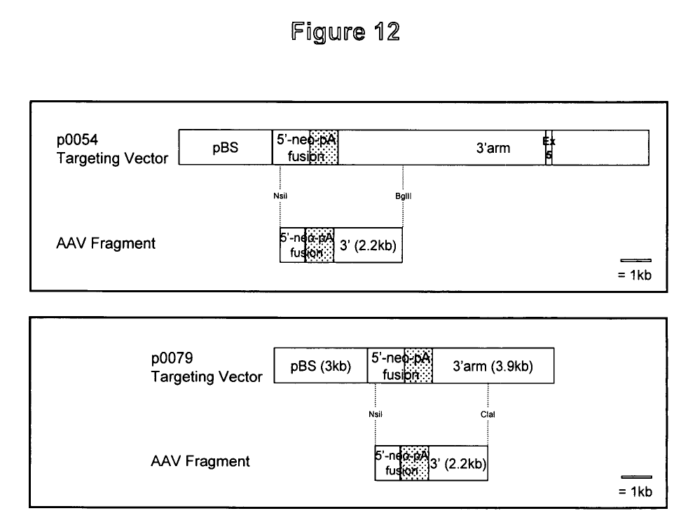

Vectors p0054, p0079, and p0063 (FIGS. 9, 10, and 11) are targeted to eliminate Exon 4, which contains the α1,3GT translation start codon. Vector p0078 (FIG. 12) is targeted to eliminate Exon 8, and contains an additional feature of interest. The sequence replacing Exon 8 has an encoding region for a poly-glycine linker, followed by the neo gene. Since neo is translated within the sequence of the rest of the α1,3GT protein, the linker should improve the flexibility or accessibility of the neo gene product and enhance the neomycin resistance conferred. Vector p0047 and p0046 are designed to replace Exon 9, which is thought to contain at least part of the α1,3GT catalytic site.

All these vectors, and other designs that would be effective in inactivating ovine α1,3GT, can be constructed using the sequence data provided in this disclosure and optionally the deposited phage plasmids, according to standard protocols of genetic engineering. Also suitable are insertion vectors (the general structure of which is reported, for example, by Hasty et al., Mol. Cell Biol. 11: 4509, 1991). In this case, homologous recombination results in insertion of the linearized vector sequence, thereby disrupting the gene. Also suitable are shorter oligonucleotides, such as chimeroplasts (A. Cole-Strauss et al., Science 273:1386, 1996), which can direct single base pair changes to a specific gene sequence.

The targeting vectors can be used to inactivate α1,3GT in sheep cells, or the cells of other phylogenetically related species that have sufficient identity through the targeted area that homologous recombination will take place. Of particular interest are ruminants of the subfamily caprinae, especially ovids and pseudois, exemplified by the sheep of any domestic breed.

Example 4 provides a non-limiting illustration of the use of such vectors. A suitable cell line is combined with the vectors in a culture medium, and the vectors are introduced into the cell. In the illustration, the vectors are introduced by optimized conditions of electroporation. The cells are then cultured for a time in an appropriate medium for maintenance of the cells, during which time the recombination event should occur. The cells are then subjected to culture conditions that permit outgrowth of cells bearing the selectable marker as a result of successful recombination.

Those skilled in the art will recognize that other means of introducing the targeting vector into the cell are also acceptable. For example, International Patent Application WO 29/48005 reports a strategy for modifying a gene in a cell by introducing a parvoviral vector comprising a sequence substantially identical to the target except for the modification being introduced, and permitting the viral vector to effect homologous recombination, thereby modifying the gene sequence. FIG. 12 shows targeting constructs based on vectors p0054 and p0079, modified to ˜4-kB, so that they will fit within the genome of adeno-associated virus. U.S. Pat. No. 5,932,241 describes cationic lipid DNA complexes for gene targeting. U.S. Pat. No. 5,804,413 describes herpes simplex virus strains for gene transfer.

After genetic manipulation has been completed and altered cells have been selected, inactivation of the α1,3GT gene can be confirmed by suitable testing. Assays for expression of α1,3GT at the mRNA level and at the protein level have already been described. The nature of the genetic alteration can be determined by PCR amplification using primers bracketing the targeted recombination site, and characterizing the amplification product, or by Southern analysis. If the targeting vector contains a unique sequence, then correct integration can be confirmed using a primer specific for the inserted sequence and a primer specific for neighboring α1,3GT sequence. Sequence information disclosed in this application relating to α1,3GT unencoding and intron regions (SEQ. ID NOs:15-25) can be used to develop such primers. As illustrated in Example 4, production of amplification product of the predicted size confirms correct integration.

Animals and Cells that are Homozygous for Inactivated α(1,3)Galactosyltransferase

Certain aspects of this invention relate to animals and tissues in which the α1,3GT gene of at least one haplotype is inactivated. To produce such animals, a cell with this characteristic is provided that has the potential to develop into a fertile embryo. In mice, it is possible to inactivate α1,3GT in embryonic stem cells, inject the cells into a blastocyst in culture, and then engraft the blastocyst into a female which acts as a surrogate to bring the genetically altered embryo to term (U.S. Pat. No. 5,849,991).

In ungulate animals, a different procedure has been developed. First, an α1,3GT gene is inactivated in cultured cells, and then the nucleus of such donor cells is transferred into an enucleated recipient cell, such as an oocyte or other pluripotent cell, that is capable of developing into a fertile embryo after the nucleus has been transferred.

International Patent Application WO 97/07669 (Roslin Institute) describes quiescent cell populations for nuclear transfer. International Patent Application WO 97/07668 (Roslin Institute) describes inactivated oocytes as cytoplast recipients for nuclear transfer. International Patent Application WO 99/01164 (University of Massachusetts) relates to cloning pigs using donor nuclei from differentiated cells. U.S. Pat. No. 5,994,619 (University of Massachusetts) reports production of chimeric bovine or porcine animals using cultured inner cell mass cells. U.S. Pat. No. 6,011,197 (Infigen) relates to a method for cloning bovines, by reprogramming non-embryonic bovine cells using leukocyte inhibitory factor (LIF) and fibroblast growth factor (FGF), then transferring the nucleus into an enucleated oocyte. International Patent Application WO 99/21415 (Stem Cell Sciences) reports nuclear transfer for production of transgenic animal embryos. Loi et al. (Reprod. Nutr. Dev. 38:615, 1998) discuss embryo transfer and related technologies in sheep reproduction. Wells et al. (Biol. Reprod. 57:385, 1997) report production of cloned lambs from an established embryonic cell line. Liu et al. (Mol. Reprod. Dev. 47:255, 1997) discuss the effect of cell cycle coordination between nucleus and cytoplasm and the use of in vitro matured oocytes in nuclear transfer in sheep embryos. Campbell et al. (Nature 380:65, 1996) report sheep cloned by nuclear transfer from an established cell line.

In the practice of this invention, donor cells are typically nucleated cells of the desired species that can be maintained easily in culture conditions. An illustrative example is a primary fibroblast line, although other types of cells (such as a stable endothelial cell culture) may also be suitable. For the production of α1,3GT inactivated sheep, suitable donor cells can be taken from fetal tissue of the Black Welsh Mountain or Finn Dorset line. The selected breed need not be isogenic for the particular vector constructs illustrated in this disclosure, because there will be sufficient identity within the α1,3GT gene to be successfully targeted. Primary fibroblast cultures can readily produced from fetuses collected at about 35 days of gestation, and subjected to a mild trypsin/EDTA solution. Cells are then introduced with the targeting vector, cultured long enough to permit genetic alteration to take place, and then selected for proper recombination, as described in the previous section.

Nuclear transfer is particularly effective if the nucleus of the donor cell is quiescent, which can be achieved by culturing the donor cell in a serum-free medium (WO 97/07669). In an exemplary method, the nucleus of a donor cell is transferred into an oocyte that is arrested in the metaphase of the second meiotic division, and subsequently activating the reconstituted cell. Briefly, unfertilized metaphase II oocytes are collected as follows: Female animals are synchronized using progestagen sponges for ˜14 days, and induced to superovulate with single injections of follicle-stimulating hormone on two successive days. Ovulation is induced with a suitable dose of gonadotrophin-releasing hormone or an analog thereof (e.g., ˜8 mg GnRH Receptal™, Hoechst, UK) on the following day. The oocytes are recovered by flushing from the oviduct one day later, washed, and enucleated by treating with cytochalasin B and aspirating the nucleus using a glass pipette. Enucleated oocytes are then placed into contact with a single cell that acts as the nucleus donor.

Fusion of the donor nucleus into the enucleated recipient cell is effected by placing the couplet in a fusion chamber and aligning it between the electrodes. Electrical pulses are then applied to induce fusion, typically a low-voltage AC pulse for several seconds, followed by a plurality of very short high-voltage DC pulses. Following an incubation period, activation is induced by application of an additional electrical pulse. The reconstructed zygote is then cultured for a time before engrafting into a surrogate female. Further details and alternative procedures are described in the patent publications cited above.

Estrus in the surrogate female is typically synchronized artificially using a suitable combination of inducing agents. Cameron et al. (Aust. Vet. J. 66:314, 1989) discuss synchronization methods and other practical aspects for commercial embryo transfer in pigs. Blum-Reckow et al. (J. Anim. Sci. 69:3335, 1991) report experiments relating to transfer of pig embryos after long-term in vitro culture. Replacing medium every 12 h during culture improved survival, and pregnancy rate improved if the sexual cycle of recipients was 24 h behind that of the donor.

The embryos are introduced into the uterus of the recipient female using any suitable technique, including devices adapted for the purpose, or appropriate surgical methods. For example, U.S. Pat. No. 4,326,505 describes surgical procedures for embryo transplants in animals, in which the uterine horn is positioned in the peritoneal cavity proximate to the vaginal wall, a cannula is inserted through the vaginal wall and into the uterine horn, and the embryo is introduced through the cannula. Non-surgical methods include using a suitable device to manipulate the injection port through the folds of the cervix to the bifurcation of the uterus. For example, devices and techniques for porcine non-surgical embryo transfer are reported by Li et al. (J. Anim. Sci. 74:2263, 1996). Wallenhorst et al. (J. Anim. Sci. 77:2327, 1999) describe the effect of transferring pig embryos to different uterine sites.

The expression of the Galα(1,3)Gal epitope is autosomal dominant, and prevention of Galα(1,3)Gal mediated xenograft rejection is improved with homozygous inactivation of α1,3GT. One option is to select cells for production of embryos that are already homozygously inactivated. For example, the α1,3GT gene can be targeted for homologous recombination with a construct that incorporates a drug resistance gene, such as neo. Targeted cells are first cultured with neomycin at a level that will select for recombination of at least one haplotype. Selected cells are then cultured at a higher level of neomycin to select double recombinants (U.S. Pat. No. 5,589,369). In another example, the α1,3GT gene is targeted simultaneously or sequentially with two different constructs for the same region, each having a different drug resistance gene. The Exon 4 targeting vectors p0054 (neo) (FIG. 9) and p0063 (pac) (FIG. 11) can be used in this capacity. Cells are then selected by culturing with the two selection drugs simultaneously or sequentially to select double recombinants. In yet another example, targeting vectors are used that delete part of the α1,3GT gene sequence, targeted cells are cloned, and their genomic DNA is subjected to Southern or PCR analysis to determine whether the sequence has been deleted from both haplotypes. In a further example, the vectors are used to target cells that actively synthesize and present on the plasma membrane a high density of Galα(1,3)Gal (such as endothelial cells). After targeting, the cells are subject to multiple rounds of proliferation and selection using specific antibody to the determinant, for example, by flow cytometry or complement-mediated lysis. The cell population surviving repeated selection will be incapable of synthesizing the xenogeneic oligosaccharide, and will be enriched for cells having homozygous inactivation of α1,3GT.

Animals homozygous for inactivated α1,3GT can also be obtained by first producing animals heterozygous for inactivated α1,3GT according to this disclosure—and then mating the heterozygous knockouts once they reach sexual maturity, to obtain progeny that have inherited inactivated α1,3GT from both parents. Alternatively, cells from a heterozygous knockout can be targeted with an inactivation vector according to this invention to inactivate α1,3GT on the other haplotype, and used to produce an embryo with homozygously inactivated α1,3GT.

Tissue Devoid of Galα(1,3)Gal Determinants

Tissue samples can be tested to determine whether α1,3GT has been successfully inactivated. As described elsewhere in this disclosure, changes to the α1,3GT gene can be determined by Southern analysis or PCR amplification of genomic DNA; mRNA expression can be determined by Northern analysis or reverse PCR, and protein expression can be determined by immunoassay or immunohistochemistry. Elimination of mRNA or protein expression correlates with homozygous inactivation of α1,3GT, whereas quantitative reduction in mRNA or protein expression correlates with inactivation of α1,3GT on one haplotype. If inactivation of α1,3GT has been effected at the level of protein catalytic activity, then mRNA and protein levels may appear normal.

A useful test for successful α1,3GT inactivation is expression of the Galα(1,3)Gal epitope. This can be determined using an antibody or lectin specific for Galα(1,3)Gal. Since it is a self-antigen in most mammals, specific antibodies cannot easily be obtained by immunizing the usual source animals. However, purified antibody can be obtained from pooled human serum by adsorbing on an affinity column of Synsorb 115 (Chembiomed, Alberta, Canada) or D(+) melibiose (Sigma). An alternative is the “IB4” lectin from Bandeiraea (Griffonia) simplicifolia (Sigma Cat. L3019) which is specific for α-D-galactosyl residues (Hayes et al., J Biol Chem. 25:1904, 1976), and binds both the Galα(1,3)Gal epitope, and B blood group substance. In most mammals, Galα(1,3)Gal is expressed on red blood cells, and can be detected in a simple agglutination reaction. The antibody or lectin can be used to stain for Galα(1,3)Gal in fixed tissue or in flow cytometry analysis.

Density of Galα(1,3)Gal in a particular tissue of interest can be quantitated using a complement lysis assay. Briefly, tissue cells are suspended (by treating with trypsin or collagenase, if necessary), washed, incubated in a medium containing ⁵¹Cr for a few hours to incorporate the label, and then washed again. The labeled targets are then combined with diluted human serum as a source of both antibody and complement, and then incubated for several hours at 37° C. Release of the label correlates with density of Galα(1,3)Gal on the surface of the target cells. For further details of assays for α1,3GT inactivation and Galα(1,3)Gal determination, the reader can consult U.S. Pat. No. 5,849,991.

Once an animal has been produced with homozygously inactivated α1,3GT, there should be no synthesis of Galα(1,3)Gal by the α1,3GT gene, and the phenotype should breed true. The animal or any progeny bearing the same phenotype can be used as a source of any tissue type that is desired for xenotransplantation. Possible harvest tissue includes but is not limited to any of the following: whole organs, such as kidney, liver, heart, lung, eyes, and pancreas; solid tissue, such as skin, cartilage, pancreatic islets, and vasculature of various types; and cell suspensions, such as progenitor cells for regeneration of neural tissue, hepatocytes, or other cell types.

### The Following Examples are Provided as Further Non-Limiting Illustrations of Particular Embodiments of the Invention

### EXAMPLES

### Example 1

### Cloning and Characterization of the Ovine α(1,3)-galactosyl Transferase cDNA

The cDNA encoding ovine α(1,3)-galactosyl transferase (GT) was cloned using the reverse transcriptase polymerase chain reaction (PCR) amplification technique.

Two oligonucleotide primers were prepared. The sense oligonucleotide, 698007 (5′-GAGAAAATAA TGAATGTCAA AGGA-3′; SEQ. ID NO:26), included 5′ untranslated sequence and the first five codons that encode mature α1,3GT. The antisense oligonucleotide, 698006 (5′-TGATAATCCC AGCAGTATTC-3′; SEQ. ID NO:27), encoded about amino acids 336 to 342. These primers were used in combination with a Titan rt. PCR kit (Boehringer Mannheim) and total RNA (TriPure™ RNA extraction kit, Boehringer Mannheim) from Black Welsh Mountain (BW) fetal fibroblasts.

The 1035bp PCR product was cloned into pCR-BluntII-TOPO vector (Invitrogen) and the resulting plasmid propagated by transforming TOP10 competent cells. One colony with plasmid containing the correct fragment was selected. Plasmid p0010 was isolated and the partial cDNA insert sequenced. The final 75 nucleotides of the coding sequence were obtained from phage G (see Example 3), giving a complete sequence of 1110 base pairs.

The 5′ untranslated region (UTR) of the ovine α1,3GT gene was cloned by rapid amplification of cDNA ends (RACE) PCR. A 5′ race kit (Boehringer Mannheim) was used in accordance with the manufacturer's instructions. Total RNA was isolated from Black Welsh Mountain fetal fibroblasts and a reverse transcriptase reaction carried out. An anchor of known sequence was linked to the 5′ termini of the cDNA molecules produced. First round PCR was performed with sense (supplied) and antisense 798006 (5′-CTTGATGGGT TTATCCAGAA CA-3′; SEQ. ID NO:28) primers. Second round PCR product, produced with the same sense primer but a nested antisense primer (798005, 5′-CTGTGGATAT ATTCCCAAAA CAC-3′; SEQ. ID NO:29), was cloned into pCR-BluntII-TOPO vector. Colonies that contained plasmid with insert were selected. One clone was sequenced and extended the ovine sequence by 193 bp into the 5′UTR (SEQ ID NO:14).

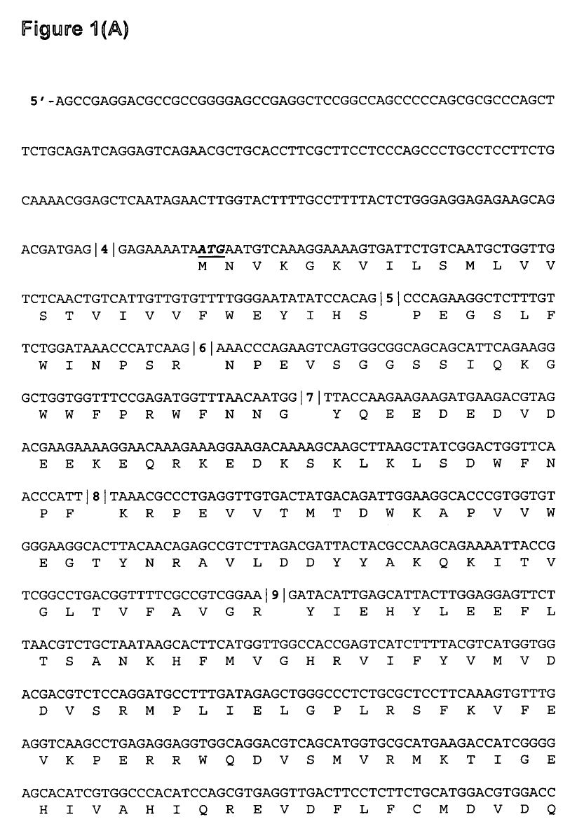

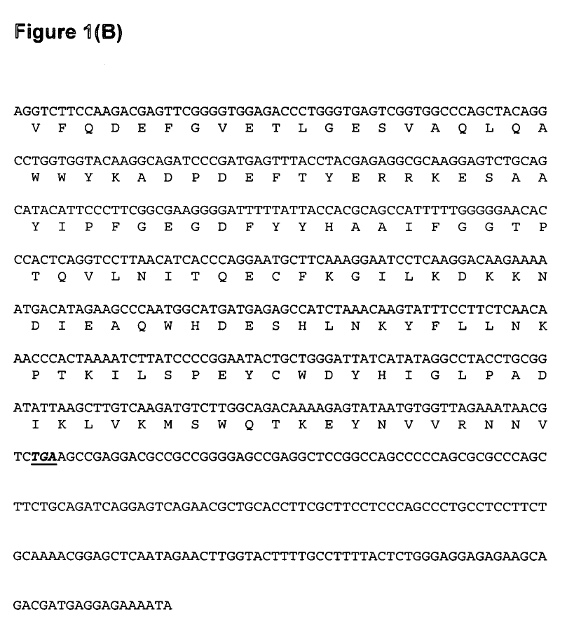

FIG. 1 shows the nucleotide sequence of the cDNA (SEQ. ID NO:1). There are 193 bp at the 5′ end that are untranslated. Also shown is the 369 amino acid sequence of the predicted protein product (SEQ. ID NO:2). The encoding region is residues 194 to 1300 of SEQ. ID NO:1.

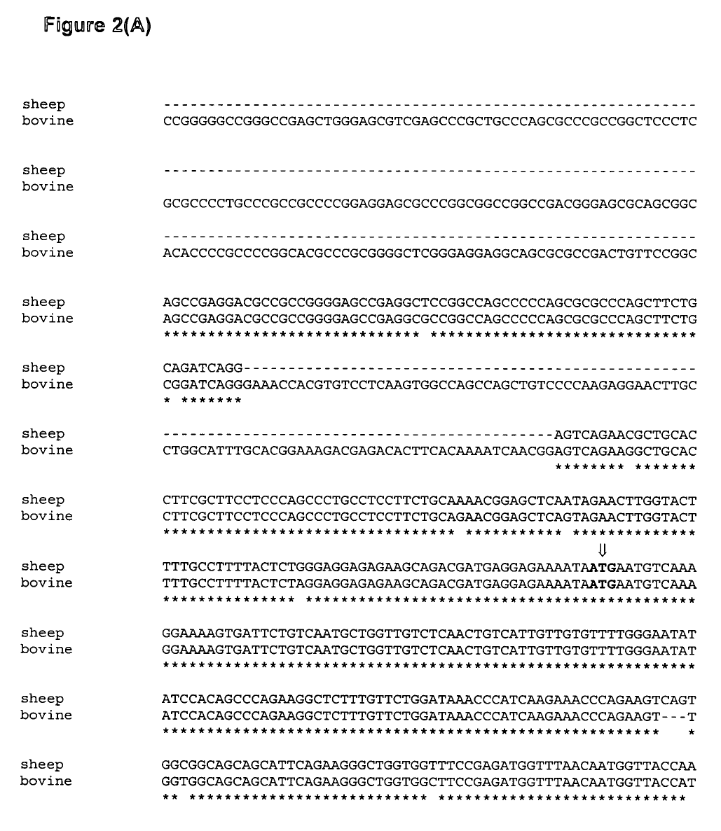

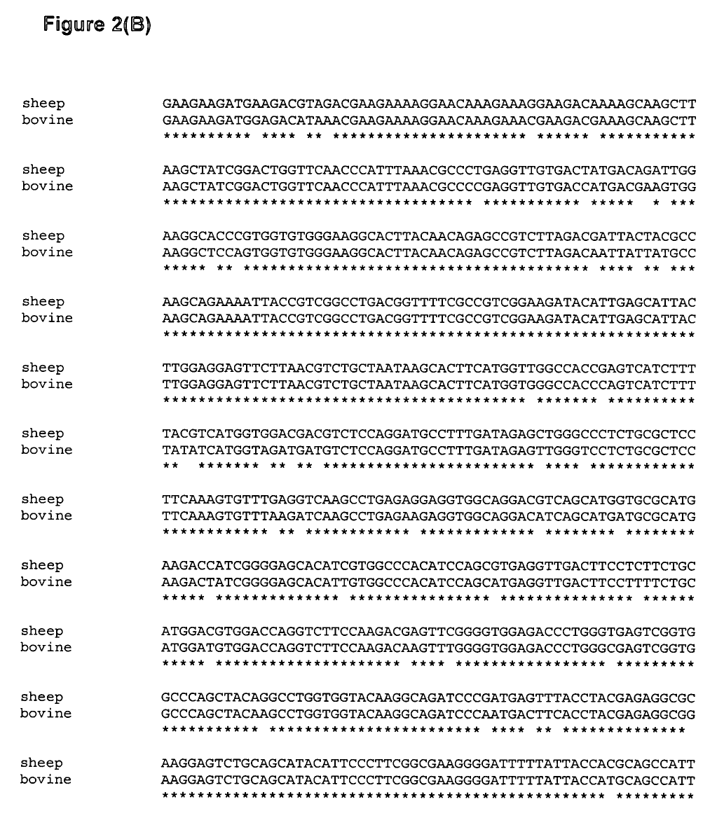

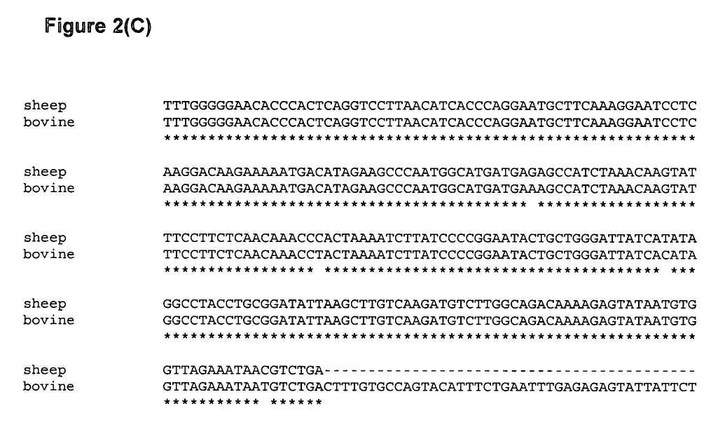

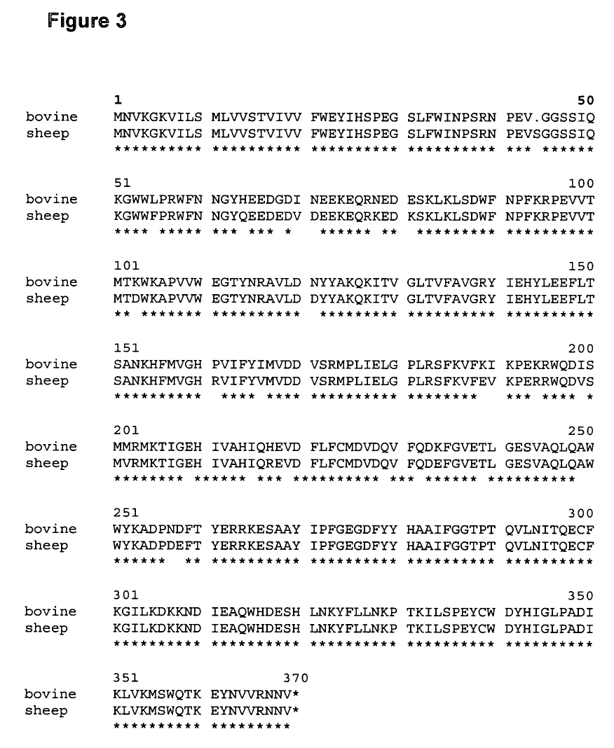

FIGS. 2 and 3 compare the polynucleotide and amino acid sequences with those of the bovine α1,3GT (SEQ. ID NOs:3 and 4). Identical residues are marked below with an asterisk.

The sheep α1,3GT encoding sequence and translation product were compared with SEQ. ID NOs 3-11 using version 2.0 of the BLASTN, BLASTP, and TBLASTN algorithms (National Center for Biotechnology Information; Altschul et al., Nucleic Acids Res. 25:3389, 1997). Comparison of ovine α1,3GT protein encoding sequence across the whole length of the murine sequence (Jozaisse et al, 1992. J Biol Chem 267:5534-5541) and bovine sequence (Joziasse et al, 1989. J Biol. Chem 264:14290-14297) showed homologies of 78% or 95% respectively. A more detailed comparison is shown in Table 1.

TABLE 1
Comparison of Sheep α1,3GT with other α1,3GT sequences

| | | Sheep α1,3GT | Percent |
|---|---|---|---|
| | | residues | identity in |
| Comparison with: | | matched | matched sequence |
| NUCLEOTIDES | | | |
| bovine | SEQ. ID NO: 3 | 194-1303 | 95% |
| α1,3GT | | | |
| marmoset | SEQ. ID NO: 5 | 448-1303 | 87% |
| α1,3GT | | | |
| pig α1,3GT | SEQ. ID NO: 7 | 503-1303 | 90% |
| mouse α1,3GT | SEQ. ID NO: 9 | 657-1303 | 83% |
| human α1,3GT | SEQ. ID NO: 11 | 620-1303 | 87% |
| pseudogene | | | |
| AMINO ACIDS | | | |
| bovine | SEQ. ID NO: 4 | 1-369 | 94% |
| α1,3GT | | | |
| marmoset | SEQ. ID NO: 6 | 1-369 | 82% |
| α1,3GT | | | |
| pig α1,3GT | SEQ. ID NO: 8 | 1-369 | 82% |
| mouse α1,3GT | SEQ. ID NO: 10 | 1-369 | 72% |
| human α1,3GT | SEQ. ID NO: 12 | 139-249 | 81% |
| pseudogene | | | |

### Example 2

### Expression and Southern Blot Analysis of the Galactosyl Transferase Gene

Based on the comparison of the ovine cDNA sequence with the genome and mouse sequence, predictions were made about the organization of the ovine α1,3GT gene. Hybridization probes for the α1,3GT gene outside the coding region were designed as follows: A partial cDNA probe, produced by excising a 1035 bp fragment from plasmid p0010 (Example 1); an Exon 4 probe, produced by concatamerizing 95 bp synthetic oligonucleotides (sense 998003, antisense 998004; SEQ. ID NOs:30 and 31); a 700 bp fragment spanning from Exon 6 to 7, derived by PCR from phage G (Example 3) using primers 898003 (5′-GGTGGTTTCC GAGATGGTTT AACA-3′; SEQ. ID NO:32) and 898005 (5′-GGGTTGAACC AGTCCGATAG CTTA-3′; SEQ. ID NO:33); a 513 bp fragment from Exon 9, produced by PCR using primers 698001 (5′-TCCAGGATGC CTTTGATAGA G-3′;SEQ. ID NO:34) and 698006 (5′-TGATAATCCC AGCAGTATTC-3′; SEQ. ID NO:27). These probes were used for the analysis in this example, and for library screening (Example 3).

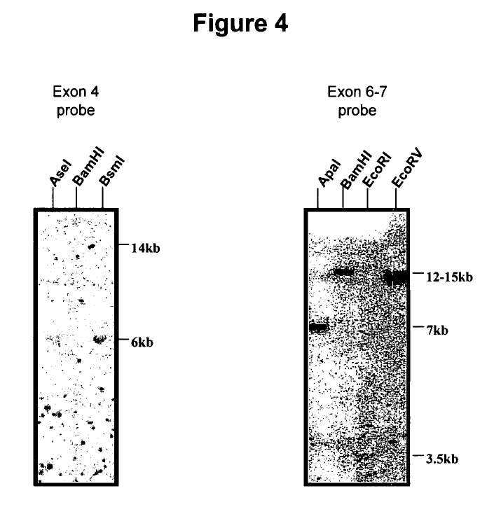

FIG. 4 shows Southern blot analysis to determine the copy number of. α1,3GT in sheep. Ovine genomic DNA was digested with ApaI, AseI, BamHI, BsmI, EcoRI or EcoRV, then transferred to nitrocellulose membrane, using standard techniques (Sambrook et al, 1989). Probing with Exon 4 or Exon 6-7 probes revealed a single band in each lane. The likelihood of achieving disruption of the ovine α1,3GT gene using the targeting strategies in Example 3 is greater if the gene is transcribed and exists as a single copy in the genome.

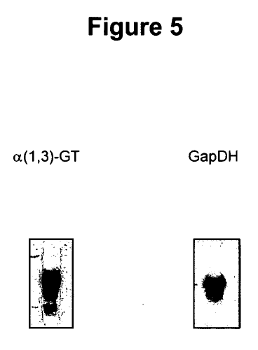

FIG. 5 shows Northern blot analysis for gene expression. Northern analysis was performed using 8 μg of Black Welsh Mountain (BW) fetal fibroblast-derived poly-A RNA, which was isolated using standard techniques (Sambrook et al, 1989). Probing with the α1,3GT partial cDNA fragment revealed a single band of approximately 3-kB, similar to that previously reported in the mouse (Jozaisse et al, 1992. J Biol Chem 267:5534-5541).

Together, these data provide evidence that the α1,3GT gene is single copy and is expressed in Black Welsh Mountain fetal fibroblasts.

### Example 3

### Construction of Targeting Vectors from Genomic Galactosyl Transferase DNA

Construction of vectors to disrupt the α1,3GT gene by homologous recombination requires isolation of genomic sequences, isogenic (or at least sufficiently identical) to sequences in the target cells. To facilitate this, genomic DNA was isolated from Black Welsh Mountain fetal fibroblasts and a λDASHII phage library constructed. Briefly, Sau3A partially digested genomic DNA was dephosphorylated and ligated to compatible BamHI vector arms (Stratagene). The ligation products were packaged to produce recombinant phage, which were propagated on spi selective XL1-Blue-MRA(P2) bacterial cells. The resulting library had a complexity of 1.4×10⁶ recombinants and was subsequently amplified once.

Using the α1,3GT Exon-4, Exon-6-7 and Exon-9 probes described in Example 2, phage clones spanning these regions were isolated according to standard methods (Sambrook et al, 1989). Six useful clones were isolated.

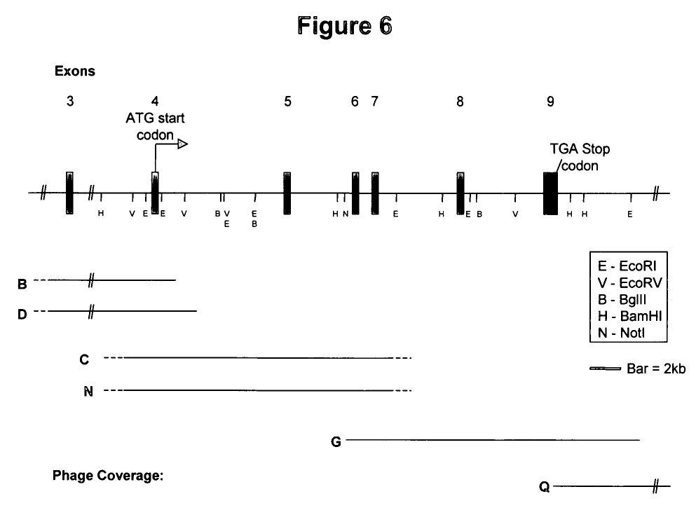

FIG. 6 shows the restriction map of the α1,3GT gene, and the regions included in Phage α1,3GT clones. The clones designated B and D contain the 5′ UTR of the α1,3GT gene and Exon 4, which contains the start codon. The clones designated C and N contain Exons 4, 5, 6 and 7. The clone G spans Exon 6, 7, 8 and 9. Clone Q contains part of Exon 9 and the 3′ UTR. These phage DNAs reflect the endogenous α1,3GT gene. They were characterized by inter-exon PCR, sequencing and restriction analysis combined with exon-specific probing.

Recombinant phage B, C and G have been deposited as a pooled sample, which can be separated using the oligonucleotide probes 1198001 (5′-GGGAGGAAGC GAAGGTGCA-3′; SEQ. ID NO:35), 798006 (5′-CTTGATGGGT TTATCCAGAA CA-3′; SEQ. ID NO:28) and 698006 (5′-TGATAATCCC AGCAGTATTC-3′; SEQ. ID NO:27), that recognize 5′UTR, Exon 5 and Exon 9 sequence, respectively.

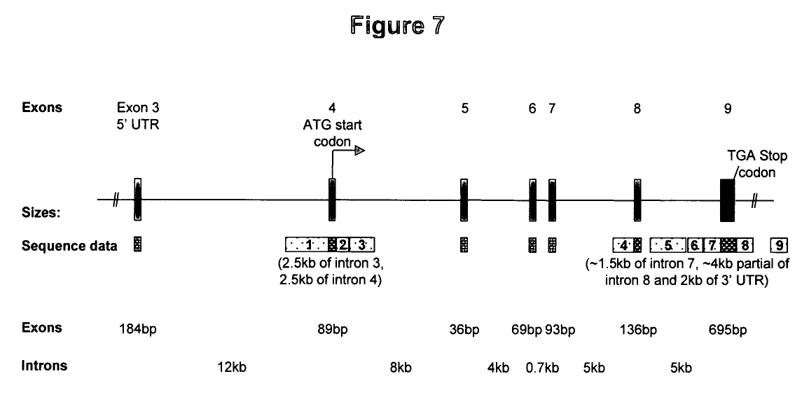

FIG. 7 maps the sequenced intron regions of α1,3GT to their positions in the gene. The areas numbered 1 to 9 in the figure correspond to SEQ. ID NOs:15, 16, 19, 20, 21, 22, 23, 24, and 25, respectively.

FIG. 8 shows the position of exemplary targeting vectors designed for use in homologous recombination to disrupt gene expression by excising Exons 4 and 9. These and a number of other vectors are described below.

FIG. 9 shows in more detail the promoterless targeting vector designated plasmid p0054, directed towards Exon 4. The vector comprises two regions that are complementary to genomic sequence; a 1.2-kB 5′ arm, which includes sequence from Intron 3 leading up to and including the start codon of the α1,3GT gene in Exon 4, and a ˜9-kB 3′ arm that initiates 1.5-kB into Intron 4, continuing to Intron 5. Separating these regions is neo^{R}-polyA sequence. After homologous recombination the vector confers neomycin phosphotransferase resistance to the cells and deletes 1.5-kB of genomic sequence, including the first coding exon of α1,3GT gene. The entire cassette was cloned into pBlueScript™ for propagation in DH5α bacterial cells.

The p0054 vector was constructed by amplifying a truncated left arm (300 bp, includes EcoRI site) (using primers 199001, 5′-ACGTGGCTCC AAGAATTCTC CAGGCAAGAG TACTGG-3′, SEQ. ID NO:36; and 199006, 5′-CATCTTGTTC AATGGCCGAT CCCATTATTT TCTCCTGGGA AAAGAAAAG-3′, with tail complementary to the start of neo coding sequence, SEQ. ID NO:137), and a neo-polyA sequence obtained from Stratagene (using primers 199005, 5′-CTTTTCTTTT CCCAGGAGAA AATAATGGGA TCGGCCATTG AACAAGATG-3′, SEQ. ID NO:38, with tail complementary to left arm; and 199004, 5′-CAGGTCGACG GATCCGAACA AAC-3′, SEQ. ID NO:39). These fragments were used to prime from each other to give a 1.2-kB fusion product. This was ligated to Intron 3 sequence, to extend the left arm, and to ˜9-kB of 3′ sequence to create the right arm, which initiates 1.5-kB into Intron 4, continuing to Intron 5.

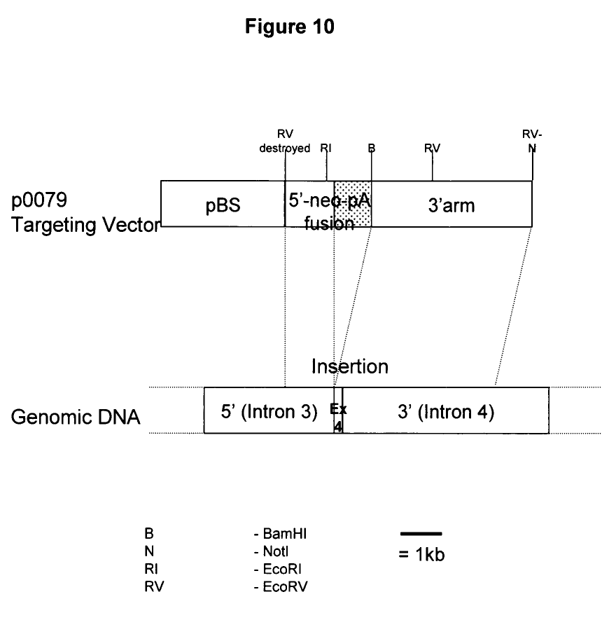

FIG. 10 shows the promoterless neo-polyA insertion vector designated plasmid p0079, also directed towards Exon 4. This vector contains the same left arm-neo-polyA fusion as in vector p0054, but with a modified right arm of 3.9-kB. The 3′ region comprises a 1.5-kB fragment, generated by PCR (using primers 200011, 5′-CAGATCTAAC GAGGATTCAA TGTCAAAGGA AAAGTGATTC TGTCAAT-3′, SEQ. ID NO:40; and 499006, 5′-CTGAACTGAA TGTTTATCCA GGCCATC-3′, SEQ. ID NO:41), which extends from the second codon in Exon 4 into Intron 4, replacing the sequence deleted in p0054. The 3′ arm was extended by ligation to a 2.4-kB EcoRV downstream fragment.

FIG. 11 shows the promoterless pac-polyA replacement vector designated plasmid p0063, also directed towards Exon 4. Construction of this vector was similar as for p0054, except that it contains the pac gene, which codes for puromycin N-acetyltransferase, rather than the neo gene. The pac sequence is available in plasmid pPUR™ from Clontech. The oligonucleotide primers used to generate the 5′-pac-polyA fusion were, for the 5′ region, 199001 (SEQ. ID NO:36) and 699002 (5′-GCGCACCGTG GGCTTGTACT CGGTCATTAT TTTCTCCTGG GAAAAGAAAA G-3′, SEQ. ID NO:42), with tail complementary to the start of pac coding sequence; and, for pac-polyA, 699004 (5′-GAGAAAATAA TGACCGAGTA CAAGCCCACG GTGC-3′ SEQ. ID NO:43), with tail complementary to left arm, and 699005 (5′-CTGGGGATCC AGACATGATA AGATAC-3′ SEQ. ID NO:44).

FIG. 12 shows restriction fragments of the Exon 4 directed targeting vectors (3.9-kB NsiI/BgIII, from p0054; 3.6-kB NsiI/ClaI, from p0079; FIG. 10). These fragments are of appropriate size for insertion into recombinant adeno-associated virus (AAV) for gene targeting according to the general approach outlined in International Patent Application WO 98/48005.

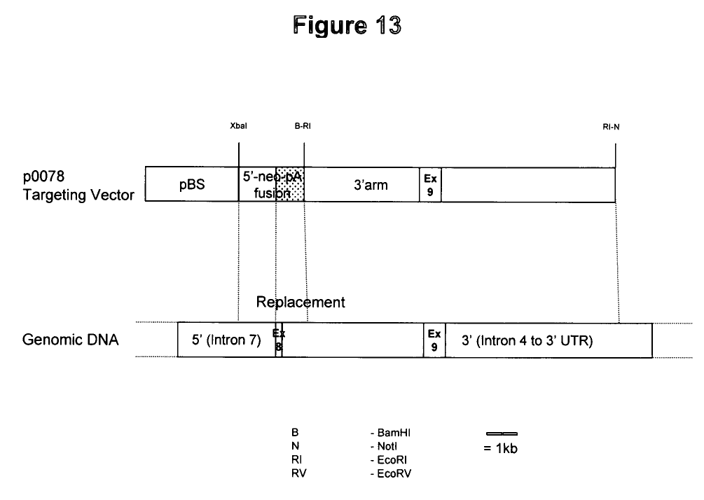

FIG. 13 shows the promoterless neo-polyA replacement vector designated plasmid p0078, directed towards Exon 8. The vector comprises a 1.3-kB left arm, which is complementary to sequence from Intron 7 up to and including the first nucleotide of Exon 8, fused in frame to 5×GGA (glycine) repeats, then to neo-polyA sequence (obtained from Stratagene). The glycine linker is designed to permit more independent rotation of each functional domain (the remaining fragment of α1,3GT, and neo), allowing neo to perform its function. The vector was constructed by amplifying the left arm (using primers 200-005, 5′-CTGGTTGGTT CTAGAACAGG AGGA-3′, SEQ. ID NO:45; and 200-007, 5′-CATCTTGTTC AATGGCCGAT CCCATTCCTC CTCCTCCTCC ACTGGTGACA AAACAGAGTC CATGAG-3′ SEQ. ID NO:46, with tail complementary to the start of neo coding sequence) and neo-polyA sequence (using primers 200-006, 5′-CTCATGGACT CTGTTTTGTC ACCAGTGGAG GAGGAGGAGG AATGGGATCG GCCATTGAAC AAGATG-3′, SEQ. ID NO:47, with tail complementary to left arm; and 200-008, 5′-CAGGTCGACG GATCCGAACA AAC -3′, SEQ. ID NO:48). These fragments were then used to prime from each other, giving a 2.2-kB fusion product. To complete the targeting vector, this product was ligated to a ˜9-kB right arm, which initiates 0.7-kB into Intron 8, continuing to the 3′UTR.

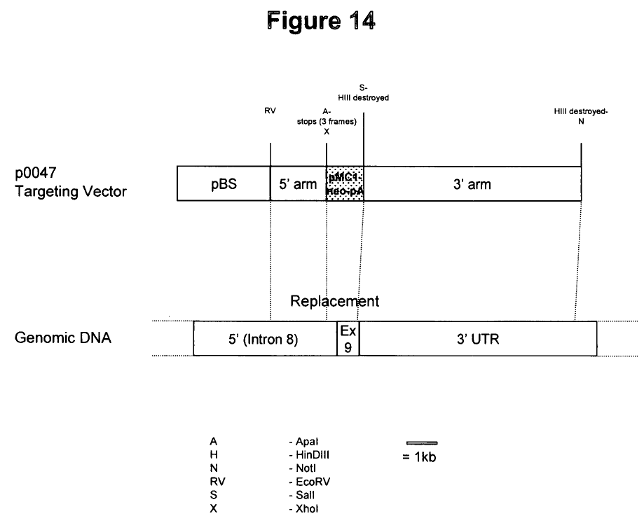

FIG. 14 shows the targeting vector designated plasmid p0047, designed to disrupt Exon 9 of the α1,3GT gene. The pMC1-neo-polyA replacement vector comprises (from left to right) 1.8-kB of Intron 8 sequence, located 0.4-kB prior to Exon 9; pMC1-neo-polyA sequence (Stratagene); and a ˜7-kB right arm (HinDIII restriction sites blunt ended using T4 DNA polymerase obtained from Promega), initiating 60 bp upstream of the stop codon of Exon 9, and continuing into the 3′UTR.

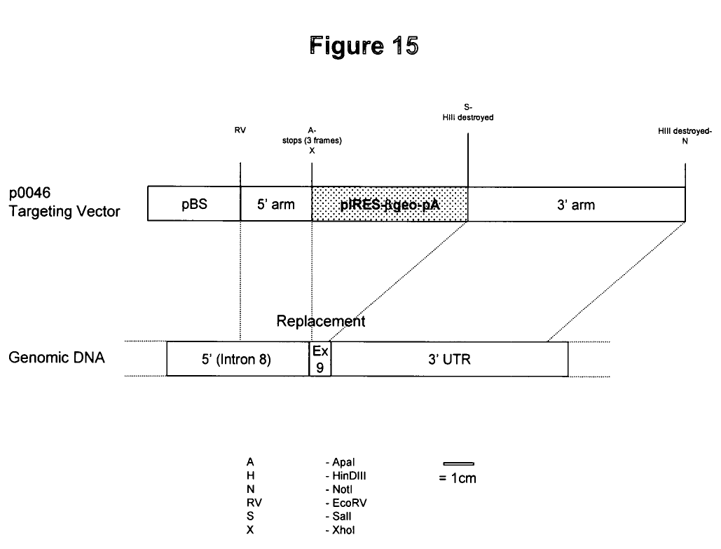

FIG. 15 shows the targeting vector designated plasmid p0046, also directed towards Exon 9. The pIRES-βgeo-polyA vector (obtained from Austin Smith, Centre for Genome Research, Edinburgh UK) contains the same right arm as p0047 extended to include the remainder of Intron 8 and the first ˜120 bp of Exon 9, which allows expression of the inserted selectable cassette: the internal ribosome entry site-β-galactosidase-neo^{R}-polyA.

After homologous recombination, the selectable marker in both p0047 and p0046 are designed to replace >80% of Exon 9, which is hypothesized to comprise at least part of the catalytic domain of α1,3GT.

### Example 4

### Disruption of the Galactosyl Transferase Gene by Homologous Recombination

Black Welsh Mountain fetal fibroblasts have previously been used in nuclear transfer experiments, creating live offspring (GB Patents 2318578 and 2331751). For the purposes of the present experiment, electroporation conditions were optimized using the β-galactosidase marker plasmid, pCMV-Sport-βgal (obtained from Gibco). Using a 0.4 cm cuvette with 3×10⁵ cells (0.8 ml, 6 μg plasmid DNA) and a setting of 250 μF: 400 Volts (Gene Pulser, BioRad), 10-30% of the surviving fibroblasts stained positive for β-gal expression.

For targeting electroporations 10, 25 or 100 μg of NotI linearized p0054 vector was mixed with 1×10⁷ early passage Black Welsh Mountain fetal fibroblasts and pulsed as described above. Cells were grown on tissue culture plastic for 24 hours before G418 (300 μg/ml) was applied. After 10-14 days colonies were isolated. Half the colony cell population was expanded for analysis, while the remainder was cryopreserved for later recovery.

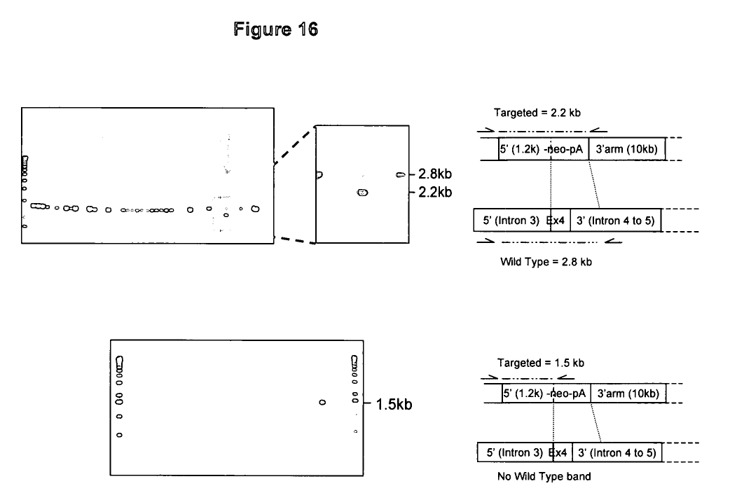

FIG. 16 shows the results of site specific recombination detected by PCR amplification. Wild type and targeted α1,3GT alleles were detected using sense (399010, 5′-CAGCTGTGTG GGTATGGGAG GG-3′; SEQ. ID NO:49) and antisense (499006, 5′-CTGAACTGAA TGTTTATCCA GGCCATC-3′; SEQ. ID NO:50) PCR primers, yielding products of 2.8-kB and 2.2-kB, respectively. A second PCR screen with primers 399010 (SEQ. ID NO:49) and 399005 (5′-AGCCGATTGT CTGTTGTGCC CAGTCAT-3′; SEQ. ID NO:51) produced a fragment of 1.5-kB only in clones that were correctly targeted. The frequency of site-specific recombination was 1 in 52 (6 in 312) clones in the 10 μg experiment or 1 in 88 (10 in 877) from all electroporations.

The neo^{R} gene was tested in one sample by Southern blot analysis. DNA was digested with the restriction enzyme BsmI, and the blot was probed with neo coding sequence, producing a diagnostic band at 5.5-kB. However, all cryopreserved samples of targeted Black Welsh Mountain cells failed to grow after resuscitation. Analysis other than PCR was not possible due to inadequate amounts of DNA. The PCR data demonstrate successful gene disruption by homologous recombination in sheep somatic cells.

Additional vectors were constructed for targeting Exon 8 or 9, using the pac gene as a resistance marker. Transfection of Black Welsh Mountain fetal fibroblasts with the pIRES-βgeo-polyA replacement vector failed to produce successful targeting in the 250 clones examined. Apparently, the efficiency of this vector was lower than the promoterless neo-polyA vector.

### Example 5

### Gene Targeting of the α1,3GT Gene in Other Breeds

In this experiment, targeting vectors were used to affect the α1,3GT gene in cells of a different breed.

Cell lines were created as follows. Finn Dorset (FD) day 35 fetuses were decapitated and eviscerated. The carcasses were dissociated manually, and then treated with trypsin/EDTA solution (Sigma). Primary cultures of fibroblasts were grown in GMEM™ media (Sigma), supplemented with 10% FCS (Globe Farm), on tissue culture plastic coated with 0.1% gelatin. Cells were exposed to gentamycin (Gibco) for the first 24 hours to prevent bacterial contamination. Once confluent, cells were cryopreserved in aliquots of ˜1×10⁶ per vial. Euploid lines of male genotype were used for gene targeting.

Cell suspensions were targeted with 10 μg of linearized promoterless neo-polyA replacement vector p0054. Electroporation was conducted by applying an electrical pulse of 125 μF, 350 Volts. A total of 568 clones were derived from one electroporated primary culture, designated 7G65F4. Colonies were screened for successful targeting as described in Example 4. Eighteen colonies (3.2%) showed patterns consistent with homogeneous cell populations in which one α1,3GT allele was deleted. A further 17 colonies containing mixed populations of targeted and non-targeted cells were detected. Surprisingly, even though the DNA of the targeting vectors is not isogenic with Finn Dorset gene sequences the targeting frequency was higher than in the experiments with Black Welsh Mountain cells (Example 4).

Homogeneous colonies of targeted FD cells stored in liquid N₂ were thawed rapidly and collected by centrifugation. Three clones (3C6, 5E1 and 4H2) from culture 7G65F4 retained a normal karyotype and fibroblast morphology, and grew successfully. Cells were grown for up to five days in normal medium containing 10% FBS, replacing the medium every 48 hours. The cells were then stressed for up to five days, in medium containing only 0.5% FBS, replacing the medium every 48 hours. Thereafter, culturing continued in 10% FBS and karyotyped. The 4H2 clone was unstable in longer periods of culture, but clones 3C6 and 5E1 grew in a slow, stable fashion, and thus suitable for nuclear donors.

### Example 6

### Animals in which an α1,3GT Gene is Inactivated

Successfully targeted fibroblast cell lines 3C6 and 5E1 are used as donors for nuclear transfer. The cells are grown for up to five days in 10% FBS, and then starved for up to five days in 0.5% FBS, as described in Example 5. Prepared cells are trypsinized for ˜5 min, and collected by low-speed centrifugation (800 rpm for 3 min). They are then resuspended in <100 μL 10% FCS medium, and used immediately for nuclear transfer.

Six weeks before the transfer, adult female breeding sheep are selected, and brought in to the facility to acclimatize to the surroundings. For each nuclear transfer experiment, about 8 animals are used as oocyte donors, 2 as temporary recipients, and 10 as final recipients.

Oocytes are collected as follows. Time of estrus is controlled in donor animals by treatment with an intravaginal progestagen sponge for 11 to 16 days, replacing once if necessary. Ovine follicular growth is promoted by injection of gonadotrophin, with or without subcutaneous or intramuscular injection of 0.6 mg (2 mL) follicle stimulating hormone (FSH) twice daily over 4 consecutive days, followed by a single injection of 500 i.u. (2.5 mL) pregnant mare's serum gonadotropin (PMSG) on the day of sponge removal. Time of ovulation is controlled by subcutaneous or intramuscular injection of an analogue of gonadotropin releasing hormone (e.g. Buserelin™, given 24 h after sponge removal). Between one and twelve days after the onset of estrus, general anesthesia is induced by an intravenous injection of a barbiturate, followed by intubation and maintenance with gaseous anesthetics. The reproductive tract is exposed by mid-ventral laparotomy, and the oviduct or uterus us is temporarily cannulated as a passageway for sterile medium. Three small puncture incisions made at sites anterior to the udder, through which a laparoscope, manipulating forceps and a needle are used to manipulate the uterus. After oocytes are collected, the incision is sutured closed, and the donor animal is allowed to recover from anesthetic.

Nuclear transfer is conducted as follows. First, the oocytes are stripped of cumulus cells by triturating with a pipette and incubating with hyaluronidase. They are then enucleated by removing the first polar body and metaphase plate. A single 3C6 or 5E1 targeted cell is introduced under the zona of each oocyte. The cell combination is subject to simultaneous electrofusion and activation (0.25 kV cm⁻¹ AC for 5 sec. to align oocyte and donor cell, followed by 3 pulses of 1.25 kV cm⁻¹ DC for 80 μsec to fuse and activate the reconstructed embryo). The activated cell is maintained in culture overnight at 39° C. Next day, the cells are embedded in agar chips to protect from macrophages, and then transferred to the ligated oviduct of a temporary recipient.

For the temporary recipient, estrous is controlled by treatment with intravaginal progestagen sponge for 11 to 16 days, with or with subcutaneous or intramuscular injection of 500 i.u. of PMSG. The timing brings the temporary recipients to estrus ˜3 days before the oocyte donors. Cells are collected under general anesthesia using barbiturate followed by gaseous anesthetics. The reproductive tract is exposed by midventral laparotomy; placing ligatures of silk at each uterotubal junction, and embryos are transferred through the fimbriated end of the oviduct. The laparotomy is then closed, and a long-acting antibiotic is administered. Blood samples of <20 mL (<5% of blood volume may be taken weekly for monitoring purposes. The embryo are flushed from the temporary recipient after 6 days, and developing embryos are removed from the agar chip.

Blastocysts and morula are then transferred into the recipients that will carry the embryo to term. Estrus is controlled by treatment with an intravaginal progestagen sponge for 11 to 16 days, bringing the final recipients to estrus simultaneously with the oocyte donor. The permanent recipients are anesthetized by intravenous barbiturate and gaseous anesthetics, the reproductive tract is exposed by mid-ventral laparotomy, and the oviduct or uterus is temporarily cannulated for transfer of the embryos. Alternatively, three small puncture incisions are made anterior to the udder, and a laparoscope, manipulating forceps and needle are inserted for manipulation of the uterus. The oviduct or uterus is temporarily cannulated for transfer of the embryos, and the incision is sutured closed.

Recipients of oocytes with a targeted nucleus, engrafted in the manner outlined, were monitored for the status of their pregnancy by subcutaneous ultrasonic scanning on a weekly basis. Ten recipients were determined to be pregnant 35 days after engraftment. At the time these observations were recorded, insufficient time had passed for any engrafted pregnancies to reach term. Some of the pregnancies were stable, and some started to regress. A fetus and umbilical cord were recovered from one of the regressing pregnancies for analysis.

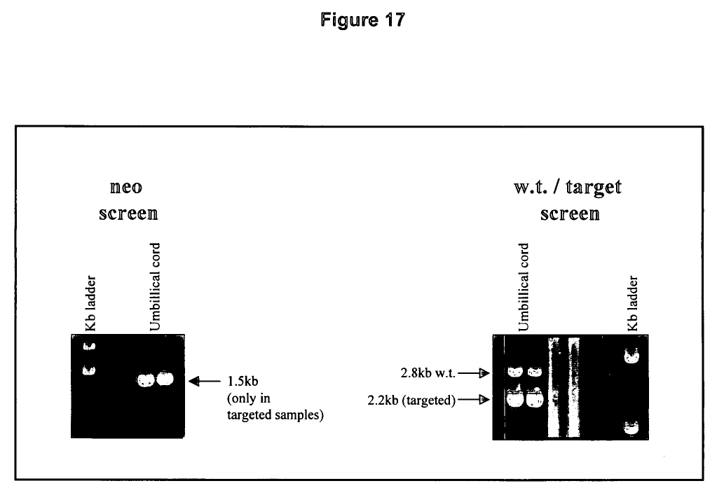

FIG. 17 shows the results of PCR analysis of the tissue from the umbilical cord. Using primers for the 5′ untranslated region and the neo gene, a band corresponding to 1.5 kB was detected, which should only be present in progeny from a cell successfully targeted with a neo-containing deletion vector. Using primers for the 5′ and 3′ untranslated regions, two bands were observed: 2.8 kB, corresponding to the wild type α1,3GT gene, and 2.2 kB, appropriate for a targeted α1,3GT gene. These results are consistent with inactivation of the α1,3GT gene on one haplotype.

For animals maintaining their pregnancy, the progress of the fetus is monitored regularly by ultrasound, and brought to term. Blood cells are collected after birth, to verify that at least one α1,3GT allele has been inactivated.

### Biological Deposit

The recombinant phage designated B, C and G, have been deposited with the National Collections of Industrial and Marine Bacteria Limited (NCIMB), 23 St. Machar Drive, Aberdeen AB2 1RY, Scotland, United Kingdom (Tel: +44 (0)1224 273332; Fax: +44 (0)1224 272461).

A deposit comprising equal concentrations of each phage was received on Apr. 25, 2000, and is catalogued under Accession No. NCIMB 41056. The concentration is 0.8×10⁸ pfu/mL for each of the three phage, for a total of 2.4×10⁸ pfu/mL. Each phage can be separated from the mixture using the following probes:

(SEQ. ID NO:35)

Phage B: probe GGGAGGAAGCGAAGGTGCA,
5′UTR

(SEQ. ID NO:28)

Phage C: probe CTTGATGGGTTTATCCAGAACA,
Exon 5

(SEQ. ID NO:27)

Phage G: probe TGATAATCCCAGCAGTATTC,
Exon 9

Each recombinant phage was deposited with the MCIMB separately on May 30, 2000. Accession numbers are: Clone B, No. 41059; Clone C, No. 41060; and Clone G, No. 41061. Each deposit has a concentration of 2×10⁸ pfu/mL.

The phage are useful for obtaining sequence information about the sheep α1,3Galactosyltransferase gene (described in Example 3) and as an amplification template for preparing certain polynucleotides of this invention.

The deposits are made under terms of the Budapest Treaty on the International Recognition of the Deposit of Microorganisms for the Purposes of Patent Procedure. For purposes of prosecution of this and any related application in the United States, the deposit is made under terms and with the undertakings of 37 CFR §§ 1.801-1.808. Subject to 37 CFR § 1.808(b), all restrictions imposed on the availability to the public of the deposited material will be irrevocably removed upon issuance of a U.S. patent thereto.

TABLE 2
Sequences listed in this Disclosure

| SEQ. ID NO: | Designation | Reference |
|---|---|---|
| 1 | Sheep α1,3-GT cDNA sequence | This Invention. |
| 2 | Sheep α1,3-GT amino acid | This Invention. |
| | sequence | |
| 3 | Bovine α1,3-GT cDNA sequence | GenBank Accession J04989 |
| | | Joziasse et al. “Bovine α1−>3-galactosyltransferase” |
| | | J. Biol. Chem. 264, 14290 (1989) |
| 4 | Bovine α1,3-GT amino acid | GenBank Accession P14769 |
| | sequence | Joziasse et al. (1989), supra. |
| 5 | Mermoset α1,3-GT cDNA | GenBank Accession S71333 |
| | sequence | lymphoid cell line B95.8, mRNA Partial |
| | | Macher et al. “Defining the minimal size of |
| | | catalytically active primate α1,3 |
| | | galactosyltransferase” Glycobiology 4, 193 (1994) |
| 6 | Mermoset α1,3-GT amino acid | GenBank Accession S71333 |
| | sequence | Macher et al., supra. |
| 7 | Pig α1,3-GT cDNA sequence | GenBank Accession L36152 |
| | | Sus scrofa alpha-1,3-galactosyltransferase mRNA. |
| | | Strahan et al. “cDNA sequence and chromosome |
| | | localization of pig α1,3 galactosyltransferase” |
| | | Immunogenetics 41, 101 (1995) |
| | | See also GenBank Accession L36535 |
| | | Sandrin et al. “Characterization of cDNA clones for |
| | | porcine a(1,3)galactosyl transferase” |
| | | Xenotransplantation (1994) |
| 8 | Pig α1,3-GT amino acid sequence | GenBank Accession L36152 |
| | | Strahan et al., supra. |
| 9 | Mouse α1,3-GT cDNA sequence | GenBank Accession M26925 |
| | | Larsen et al. “Isolation of a cDNA encoding a murine |
| | | UDP-galactose: β-D-galactosyl-1,4-N-acetyl-D- |
| | | glucosaminide alpha-1,3-galactosyltransferase” |
| | | Proc. Natl. Acad. Sci. USA 86, 8227 (1989) |
| | | See also GenBank Accession IM85153 |
| | | Joziasse et al. “Murine alpha-1,3- |
| | | galactosyltransferase: A single gene locus specifies |
| | | four isoforms of the enzyme by alternative splicing” |
| | | J. Biol. Chem. 267, 5534 (1992) |
| 10 | Mouse α1,3-GT amino acid | GenBank Accession M26925 |
| | sequence | Larsen et al., supra. |
| 11 | Human α1,3-GT pseudogene DNA | GenBank Accession M60263 |
| | sequence | Joziasse et al. “Characterization of an alpha-1−>3- |
| | | galactosyltransferase homologue on human |
| | | chromosome 12 that is organized as a processed |
| | | pseudogene” J. Biol. Chem. 266, 6991 (1991) |
| 12 | Human α1,3-GT pseudogene open | calculated from |
| | reading frame amino acid | GenBank Accession M60263 |
| | sequence | Joziasse et al., supra. |
| 13 | Bovine γ globin gene and globin | GenBank Accession M63452 |
| | pseudogene | C. H. Duncan (direct submission) |
| 14 | Exon 4 of the sheep α1,3-GT gene | This Invention. |
| 15 | Pre-Exon 4 sequence (Phage B | This Invention. |
| | and C) | Region 1 of FIG. 7 |
| 16 | Post-Exon 4 sequence | This Invention. |
| | Exon 4/intron 4 boundary | Region 2 of FIG. 7 |
| 17 | Post-Exon 4 sequence | This Invention. |
| 18 | Post-Exon 4 sequence | This Invention. |
| 19 | Post-Exon 4 sequence | This Invention. |
| | | Region 3 of FIG. 7 |
| 20 | Pre-Exon 8 sequence | This Invention. |
| | | Region 4 of FIG. 7 |
| 21 | Intron sequence approx −4 kb to | This Invention. |
| | −2 kb from Exon 9 | Region 5 of FIG. 7 |
| 22 | Intron sequence approx −2 kb to | This Invention. |
| | −1 kb from Exon 9 | Region 6 of FIG. 7 |
| 23 | Intron sequence 5′ to Exon 9 | This Invention. |
| | | Region 7 of FIG. 7 |
| 24 | Intron sequence 3′ to Exon 9 | This Invention. |
| | | Region 8 of FIG. 7 |
| 25 | Intron sequence approx −2 kb to | This Invention. |
| | −1 kb from Exon 9 | Region 9 of FIG. 7 |
| 26 to 51 | Probes and PCR primers | This Invention. |

1. An isolated polynucleotide that comprises a sequence of at least 30 consecutive nucleotides with at least one of the following properties:
a) it is contained in SEQ. ID NO:1 or any of SEQ. ID NOs:14 to 25, but not in any of SEQ. ID NOs: 3, 5, 7, 9, 11, and 13;
b) it is contained in phage “B”, “C” or “G” deposited under Accession Nos. NCIMB 41056, 41059, 41060, and 41061; but not in λ-phage or any of SEQ. ID NOs: 3, 5, 7, 9, 11, and 13; or
c) it hybridizes under stringent conditions to a polynucleotide with the sequence in SEQ. ID NO:1 or any of SEQ. ID NOs:14 to 25, but not to a polynucleotide with the sequence in any of SEQ. ID NOs: 3, 5, 7, 9, 11, and 13

2. The polynucleotide of claim 1, wherein said sequence is contained in SEQ. ID NO:1 or any of SEQ. ID NOs:14 to 25, but not in any of SEQ. ID NOs: 3, 5, 7, 9, 11,and 13.

3. The polynucleotide of claim 1, wherein said sequence is contained in phage “B”, “C” or “G” deposited under Accession Nos. NCIMB 41056, 41059, 41060, and 41061; but not in λ-phage or any of SEQ. ID NOs: 3, 5, 7, 9, 11, and 13.

4. The isolated polynucleotide of claim 1, wherein said sequence hybridizes at 65° C. in 2×SSC to a polynucleotide with the sequence in SEQ. ID NO:1 or any of SEQ. ID NOs:14 to 25, but not to a polynucleotide with the sequence in any of SEQ. ID NOs: 3, 5, 7, 9, 11, and 13.

5. The isolated polynucleotide of claim 1, which encodes a protein having galactosyltransferase activity.

6. The isolated polynucleotide of claim 1, which is a probe or primer for detecting or amplifying a galactosyltransferase encoding nucleic acid sequence in a sample.

7. An isolated polynucleotide encoding a protein comprising a sequence that is at least 90% identical to SEQ. ID NO:2, or fragment thereof that has α(1,3)galactosyltransferase (α1,3GT) activity, but which is not identical to any of SEQ. ID NOs: 4, 6, 8, 10, and 12.

8. The polynucleotide of claim 7, which encodes a protein comprising a sequence that is identical to SEQ. ID NO:2, or fragment thereof that has α1,3GT activity.

9. The polynucleotide of claim 7, which is an expression vector in which expression of the sequence encoding said protein is operatively linked to a transcription control sequence.

10. The polynucleotide of claim 7, contained in a host cell.

11. An isolated mammalian cell genetically altered to express the polynucleotide of claim 7.

12. The cell of claim 11, which forms Galα(1,3)Gal epitopes as a result of said genetic alteration.

13. The cell of claim 12, which would not otherwise express Galα(1,3)Gal epitopes.

14. The cell of claim 11, which is an embryonic cell.

15. A method for forming a Galα(1,3)Gal antigen on the surface of a cell that has an α1,3GT acceptor, comprising expressing the polynucleotide of claim 7 in the cell.

16. A method for forming a Galα(1,3)Gal antigen on the surface of a cell that has an α1,3GT acceptor, and on the progeny of said cell, comprising genetically altering the cell with an expression vector according to claim 9.

17. A method of killing a mammalian cell genetically altered to express the polynucleotide of claim 7, comprising placing the cell in an environment containing naturally occurring antibody against Galα(1,3)Gal.

18. The method of claim 17, wherein said environment comprises human plasma or serum.


SEQUENCE LISTING (51 sequences)

SEQ ID NO: 1 - DNA, 1303 bp, Ovis sp.
  CDS (194)..(1303)
agccgaggacgccgccggggagccgaggctccggccagcccccagcgcgcccagcttctgcagatcaggagtcagaacgctgcaccttcgcttcctcccagccctgcctccttctgcaaaacggagctcaatagaacttggtacttttgccttttactctgggaggagagaagcagacgatgaggagaaaataatgaatgtcaaaggaaaagtgattctgtcaatgctggttgtctcaactgtcattgttgtgttttgggaatatatccacagcccagaaggctctttgttctggataaacccatcaagaaacccagaagtcagtggcggcagcagcattcagaagggctggtggtttccgagatggtttaacaatggttaccaagaagaagatgaagacgtagacgaagaaaaggaacaaagaaaggaagacaaaagcaagcttaagctatcggactggttcaacccatttaaacgccctgaggttgtgactatgacagattggaaggcacccgtggtgtgggaaggcacttacaacagagccgtcttagacgattactacgccaagcagaaaattaccgtcggcctgacggttttcgccgtcggaagatacattgagcattacttggaggagttcttaacgtctgctaataagcacttcatggttggccaccgagtcatcttttacgtcatggtggacgacgtctccaggatgcctttgatagagctgggccctctgcgctccttcaaagtgtttgaggtcaagcctgagaggaggtggcaggacgtcagcatggtgcgcatgaagaccatcggggagcacatcgtggcccacatccagcgtgaggttgacttcctcttctgcatggacgtggaccaggtcttccaagacgagttcggggtggagaccctgggtgagtcggtggcccagctacaggcctggtggtacaaggcagatcccgatgagtttacctacgagaggcgcaaggagtctgcagcatacattcccttcggcgaaggggatttttattaccacgcagccatttttgggggaacacccactcaggtccttaacatcacccaggaatgcttcaaaggaatcctcaaggacaagaaaaatgacatagaagcccaatggcatgatgagagccatctaaacaagtatttccttctcaacaaacccactaaaatcttatccccggaatactgctgggattatcatataggcctacctgcggatattaagcttgtcaagatgtcttggcagacaaaagagtataatgtggttagaaataacgtctga

SEQ ID NO: 2 - protein, 369 aa, Ovis sp.
MNVKGKVILSMLVVSTVIVVFWEYIHSPEGSLFWINPSRNPEVSGGSSIQKGWWFPRWFNNGYQEEDEDVDEEKEQRKEDKSKLKLSDWFNPFKRPEVVTMTDWKAPVVWEGTYNRAVLDDYYAKQKITVGLTVFAVGRYIEHYLEEFLTSANKHFMVGHRVIFYVMVDDVSRMPLIELGPLRSFKVFEVKPERRWQDVSMVRMKTIGEHIVAHIQREVDFLFCMDVDQVFQDEFGVETLGESVAQLQAWWYKADPDEFTYERRKESAAYIPFGEGDFYYHAAIFGGTPTQVLNITQECFKGILKDKKNDIEAQWHDESHLNKYFLLNKPTKILSPEYCWDYHIGLPADIKLVKMSWQTKEYNVVRNNV

SEQ ID NO: 3 - DNA, 1617 bp, BOVINE
  CDS (469)..(1575)
ccgggggccgggccgagctgggagcgtcgagcccgctgcccagcgcccgccggctccctcgcgcccctgcccgccgccccggaggagcgcccggcggccggccgacgggagcgcagcggcacaccccgccccggcacgcccgcggggctcgggaggaggcagcgcgccgactgttccggcagccgaggacgccgccggggagccgaggcgccggccagcccccagcgcgcccagcttctgcggatcagggaaaccacgtgtcctcaagtggccagccagctgtccccaagaggaacttgcctggcatttgcacggaaagacgagacacttcacaaaatcaacggagtcagaaggctgcaccttcgcttcctcccagccctgcctccttctgcagaacggagctcagtagaacttggtacttttgccttttactctaggaggagagaagcagacgatgaggagaaaataatgaatgtcaaaggaaaagtgattctgtcaatgctggttgtctcaactgtcattgttgtgttttgggaatatatccacagcccagaaggctctttgttctggataaacccatcaagaaacccagaagttggtggcagcagcattcagaagggctggtggcttccgagatggtttaacaatggttaccatgaagaagatggagacataaacgaagaaaaggaacaaagaaacgaagacgaaagcaagcttaagctatcggactggttcaacccatttaaacgccccgaggttgtgaccatgacgaagtggaaggctccagtggtgtgggaaggcacttacaacagagccgtcttagacaattattatgccaagcagaaaattaccgtcggcctgacggttttcgccgtcggaagatacattgagcattacttggaggagttcttaacgtctgctaataagcacttcatggtgggccacccagtcatcttttatatcatggtagatgatgtctccaggatgcctttgatagagttgggtcctctgcgctccttcaaagtgtttaagatcaagcctgagaagaggtggcaggacatcagcatgatgcgcatgaagactatcggggagcacattgtggcccacatccagcatgaggttgacttccttttctgcatggatgtggaccaggtcttccaagacaagtttggggtggagaccctgggcgagtcggtggcccagctacaagcctggtggtacaaggcagatcccaatgacttcacctacgagaggcggaaggagtctgcagcatacattcccttcggcgaaggggatttttattaccatgcagccatttttgggggaacacccactcaggtccttaacatcacccaggaatgcttcaaaggaatcctcaaggacaagaaaaatgacatagaagcccaatggcatgatgaaagccatctaaacaagtatttccttctcaacaaacctactaaaatcttatccccggaatactgctgggattatcacataggcctacctgcggatattaagcttgtcaagatgtcttggcagacaaaagagtataatgtggttagaaataatgtctgactttgtgccagtacatttctgaatttgagagagtattattct

SEQ ID NO: 4 - protein, 368 aa, BOVINE
MNVKGKVILSMLVVSTVIVVFWEYIHSPEGSLFWINPSRNPEVGGSSIQKGWWLPRWFNNGYHEEDGDINEEKEQRNEDESKLKLSDWFNPFKRPEVVTMTKWKAPVVWEGTYNRAVLDNYYAKQKITVGLTVFAVGRYIEHYLEEFLTSANKHFMVGHPVIFYIMVDDVSRMPLIELGPLRSFKVFKIKPEKRWQDISMMRMKTIGEHIVAHIQHEVDFLFCMDVDQVFQDKFGVETLGESVAQLQAWWYKADPNDFTYERRKESAAYIPFGEGDFYYHAAIFGGTPTQVLNITQECFKGILKDKKNDIEAQWHDESHLNKYFLLNKPTKILSPEYCWDYHIGLPADIKLVKMSWQTKEYNVVRNNV

SEQ ID NO: 5 - DNA, 1131 bp, Mermoset alpha 1,3-GT
  CDS (1)..(1131)
atgaatgtcaaaggaaaagtaattctgtcgatgctggttgtctcaactgtgattgttgtgttttgggaatatatcaacagcccagaaggctctttcttgtggatatatcactcaaagaacccagaagttgatgacagcagtgctcagaaggactggtggtttcctggctggtttaacaatgggatccacaattatcaacaagaggaagaagacacagacaaagaaaaaggaagagaggaggaacaaaaaaaggaagatgacacaacagagcttcggctatgggactggtttaatccaaagaaacgcccagaggttatgacagtgacccaatggaaggcgccggttgtgtgggaaggcacttacaacaaagccatcctagaaaattattatgccaaacagaaaattaccgtggggttgacggtttttgctattggaagatatattgagcattacttggaggagttcgtaacatctgctaataggtacttcatggtcggccacaaagtcatattttatgtcatggtggatgatgtctccaaggcgccgtttatagagctgggtcctctgcgttccttcaaagtgtttgaggtcaagccagagaagaggtggcaagacatcagcatgatgcgtatgaagaccatcggggagcacatcttggcccacatccaacacgaggttgacttcctcttctgcatggatgtggaccaggtcttccaagaccattttggggtagagaccctgggccagtcggtggctcagctacaggcctggtggtacaaggcagatcctgatgactttacctatgagaggcggaaagagtcggcagcatatattccatttggccagggggatttttattaccatgcagccatttttggaggaacaccgattcaggttctcaacatcacccaggagtgctttaagggaatcctcctggacaagaaaaatgacatagaagccgagtggcatgatgaaagccacctaaacaagtatttccttctcaacaaaccctctaaaatcttatctccagaatactgctgggattatcatataggcctgccttcagatattaaaactgtcaagctatcatggcaaacaaaagagtataatttggttagaaagaatgtctga

SEQ ID NO: 6 - protein, 376 aa, Mermoset alpha 1,3-GT
MNVKGKVILSMLVVSTVIVVFWEYINSPEGSFLWIYHSKNPEVDDSSAQKDWWFPGWFNNGIHNYQQEEEDTDKEKGREEEQKKEDDTTELRLWDWFNPKKRPEVMTVTQWKAPVVWEGTYNKAILENYYAKQKITVGLTVFAIGRYIEHYLEEFVTSANRYFMVGHKVIFYVMVDDVSKAPFIELGPLRSFKVFEVKPEKRWQDISMMRMKTIGEHILAHIQHEVDFLFCMDVDQVFQDHFGVETLGQSVAQLQAWWYKADPDDFTYERRKESAAYIPFGQGDFYYHAAIFGGTPIQVLNITQECFKGILLDKKNDIEAEWHDESHLNKYFLLNKPSKILSPEYCWDYHIGLPSDIKTVKLSWQTKEYNLVRKNV

SEQ ID NO: 7 - DNA, 1269 bp, Sus scrofa
  CDS (16)..(1131)
catgaggagaaaataatgaatgtcaaaggaagagtggttctgtcaatgctgcttgtctcaactgtaatggttgtgttttgggaatacatcaacagcccagaaggttctttgttctggatataccagtcaaaaaacccagaagttggcagcagtgctcagaggggctggtggtttccgagctggtttaacaatgggactcacagttaccacgaagaagaagacgctataggcaacgaaaaggaacaaagaaaagaagacaacagaggagagcttccgctagtggactggtttaatcctgagaaacgcccagaggtcgtgaccataaccagatggaaggctccagtggtatgggaaggcacttacaacagagccgtcttagataattattatgccaaacagaaaattaccgtgggcttgacggtttttgctgtcggaagatacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcatggacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaataacatctgactttaaattgtgccagcagttttctgaatttgaaagagtattactctggctacttctccagagaagtagcacttaattttaacttttaaaaaaatactaacaaaataccaacacagtaagtacatattattcttcctt

SEQ ID NO: 8 - protein, 371 aa, Sus scrofa
MNVKGRVVLSMLLVSTVMVVFWEYINSPEGSLFWIYQSKNPEVGSSAQRGWWFPSWFNNGTHSYHEEEDAIGNEKEQRKEDNRGELPLVDWFNPEKRPEVVTITRWKAPVVWEGTYNRAVLDNYYAKQKITVGLTVFAVGRYIEHYLEEFLISANTYFMVGHKVIFYIMVDDISRMPLIELGPLRSFKVFEIKSEKRWQDISMMRMKTIGEHILAHIQHEVDFLFCMDVDQVFQNNFGVETLGQSVAQLQAWWYKAHPDEFTYERRKESAAYIPFGQGDFYYHAAIFGGTPTQVLNITQECFKGILQDKENDIEAEWHDESHLNKYFLLNKPTKILSPEYCWDYHIGMSVDIRIVKIAWQKKEYNLVRNNI

SEQ ID NO: 9 - DNA, 1500 bp, Musca sp.
  CDS (277)..(1461)
ccttcccttgtagactcttcttggaatgagaagtaccgattctgctgaagacctcgcgctctcaggctctgggagttggaaccctgtaccttcctttcctctgctgagccctgcctccttaggcaggccagagctcgacagaactcggttgctttgctgtttgctttggagggaacacagctgacgatgaggctgactttgaactcaagagatctgcttaccccagtctcctggaattaaaggcctgtactacatttgcctggacctaagattttcatgatcactatgcttcaagatctccatgtcaacaagatctccatgtcaagatccaagtcagaaacaagtcttccatcctcaagatctggatcacaggagaaaataatgaatgtcaagggaaaagtaatcctgttgatgctgattgtctcaaccgtggttgtcgtgttttgggaatatgtcaacagaattccagaggttggtgagaacagatggcagaaggactggtggttcccaagctggtttaaaaatgggacccacagttatcaagaagacaacgtagaaggacggagagaaaagggtagaaatggagatcgcattgaagagcctcagctatgggactggttcaatccaaagaaccgcccggatgttttgacagtgaccccgtggaaggcgccgattgtgtgggaaggcacttatgacacagctctgctggaaaagtactacgccacacagaaactcactgtggggctgacagtgtttgctgtgggaaagtacattgagcattacttagaagactttctggagtctgctgacatgtacttcatggttggccatcgggtcatattttacgtcatgatagacgacacctcccggatgcctgtcgtgcacctgaaccctctacattccttacaagtctttgagatcaggtctgagaagaggtggcaggatatcagcatgatgcgcatgaagaccattggggagcacatcctggcccacatccagcacgaggtcgacttcctcttctgcatggacgtggatcaagtctttcaagacaacttcggggtggaaactctgggccagctggtagcacagctccaggcctggtggtacaaggccagtcccgagaagttcacctatgagaggcgggaactgtcggccgcgtacattccattcggagagggggatttttactaccacgcggccatttttggaggaacgcctactcacattctcaacctcaccagggagtgctttaaggggatcctccaggacaagaaacatgacatagaagcccagtggcatgatgagagccacctcaacaaatacttccttttcaacaaacccactaaaatcctatctccagagtattgctgggactatcagataggcctgccttcagatattaaaagtgtcaaggtagcttggcagacaaaagagtataatttggttagaaataatgtctgacttcaaattgtgatggaaacttgacactatttctaacca

SEQ ID NO: 10 - protein, 394 aa, Musca sp.
MITMLQDLHVNKISMSRSKSETSLPSSRSGSQEKIMNVKGKVILLMLIVSTVVVVFWEYVNRIPEVGENRWQKDWWFPSWFKNGTHSYQEDNVEGRREKGRNGDRIEEPQLWDWFNPKNRPDVLTVTPWKAPIVWEGTYDTALLEKYYATQKLTVGLTVFAVGKYIEHYLEDFLESADMYFMVGHRVIFYVMIDDTSRMPVVHLNPLHSLQVFEIRSEKRWQDISMMRMKTIGEHILAHIQHEVDFLFCMDVDQVFQDNFGVETLGQLVAQLQAWWYKASPEKFTYERRELSAAYIPFGEGDFYYHAAIFGGTPTHILNLTRECFKGILQDKKHDIEAQWHDESHLNKYFLFNKPTKILSPEYCWDYQIGLPSDIKSVKVAWQTKEYNLVRNNV

SEQ ID NO: 11 - DNA, 1885 bp, Homo sapiens
tgcctcagcctcccaaagtgctggactacaggcatgagccaaccacgcccgcccagccaaatatggtgaaaccctttctctactaaaaatacaaaaattagctgggcagatgataaggagaaaataatgaatgtcaaaggaaacgtaattcctccagtgctggttgtctcaactgcgattgttgtggtttggaaatgtatcaacacccaagaaggctctctcttgtggatgtatcacttaaaacacccagaagttgttgacggcggcgggcagaagggccggtggtttccaagctgcttcaacaatgggacccacaatcctcaagcagaggaagaagaagacatagacaaagaaaaagagaaagaaaaaagaaaagaagatgacaaaaaaagagcttcggctatggaactggtttaatccatacgatcgcccagaggtgctgacggtgaccagttggaatgagccggttgtgtgggaaggcacttagaacagaaccatcctagaaaattattatgccaaacagaaaattactgtgggttgacggtttttgctgtcggaagacatgtagagtattacttgaaggagttcatagcatctgctgataggtacttcatggtaagccacaaagtcatgttttacatcatggccgataatccaggatgctgttgatagagctgggtcctctgcgtttcttcagagtgtttgagatcaagcccgagaagaggtagcaggtcatcagcatcgtgggcatgaagaccaacagggagcacatgttggcccacatccaacaggaagttgacttcctcttctgcatggatgtggaccaagtgttccaagacaattttggagtggagaccctgggccagtccgtggctcagctataggcctggtggtactggacacatcctgaggagaatacttaggagaggcagaaggagtcggtggcatacattccatttggccagggggatttttattaccacgcagccatttttggtggaacacccattcactttctcaacatcacccaggagtgctttaagggaatcctccaggacaggaaaaatgacctcgaagccaagtggcatgataaagccacctaaacaagtatttcttcctcaacataccctctaagatcttatcaccagaatgctgctgggattatcattataggtcatcatgaagatattaaaattgtcaagctatcttggcagcaaaaagaatatacgttggttagaaatgatgactgacttcaaattgtgccaacagttttttgaatttgagagaattattattctggctacttcctcagaaaacaattttaactttaagaaaatattaatgaaacaccaacattgcaatcacatactatctctccttgtaactttgagcattgtaatatgagagaatgaatctatggtaatcaggtgtaaattcccagtgatttcttatgtattctgaatttgggggaaatactatcagctgaaccagaaagaacttgtcataggcaaagataaagccagaaacaggccaggcacggcggcttatgcctgtaatcccagtactttgggaggccaagggagtggatcacctgaggtcaggagttcgagaccagcttggccaacatggtgaaatcccgtctctactaaaaatacaaaaaattagccgggcatggtggcgcacacctgtaatcccacctactcaggaggctgaggcgggagaatcacttgaacctgggaggcagaggttgcattgagccagattgtaccattgtactccagcctgggcaaaagagtgaaattccatctaaaaacaaacaaacaaaactctacatgtgtcagataacatcctggagaaaatcaggtggtaagggaagtgttggcag

SEQ ID NO: 12 - protein, 197 aa, Homo sapiens
SRMLLIELGPLRFFRVFEIKPEKRXQVISIVGMKTNREHMLAHIQQEVDFLFCMDVDQVFQDNFGVETLGQSVAQLXAWWYWTHPEENTXERQKESVAYIPFGQGDFYYHAAIFGGTPIHFLNITQECFKGILQDRKNDLEAKWHDKATXTSISSSTYPLRSYHQNAAGIIIIGHHEDIKIVKLSWQQKEYTLVRND

SEQ ID NO: 13 - DNA, 17141 bp, BOVINE
ggagagtagagtttctgagtttagacacactgaatcagccaatcacagatgaagagcactgagcaacaagagttcatcttacattcccccaaaccaatgaacttgtattatgccctgggctaatctgctctcagaagcagggagggcaggaggctgggtggggctcacaaggaagaccagggcccctactgcttacacatgcttttgacacaacttgcagctgcacaaacacacatcatggtgtatctgactcttgagaagaaggctactgtcattgacttgtggagtaagatgagggtggctgaagttggtccggatactgtaggcaggcaggtattcaacttacaaggcaggctgaaggagagtgaatgtcagttgggtgtgtggggacagagccattgcctgagattctggcaggcactgactccctctgaccttgtgctgttttcaccccctttgctgctggtcgtctacccctcgactcagaggttctttgactattgtggggactgtcctttgctgatttatgggcaatgttttaccttctttgtttccaggcatagtttcctcttattcattcttgtttttctctgtttgttctctgcagtatcttctttatatttaaacattttgactgtttaagtgtttgagtattaagactttcttcttttatgtcacttaaaaattttgtctcatgattttcccttatctcttcttttttaagcaaggaagacaaaatgatgtattgcttcttgaaacagttcaaaaaaataaaaaaaatgatagcaagttcagaattaagatagaaagagagaaacatctctaagtataaactcaggcttatatgggtggcttcacatcagtagtaacatctacactttagccatctttctgcttatattctaggggcacagcttgagatgagactgaaatactaagtccaaattgggtgcctctgctaacattgtccttgtttttcatctctaccacacagctcctgggcaatatactgatgagtacactggcttgaaactttggcaaggaattcaccccagaatttcaggttgcctgtcagaaggtggtggctggtgtggttaatgctctcacctacaaataccattgagatcctgtcctattttttattttcaaatgagtatttatttctaattgattgatgattggtttacaatattggtttgattactatcatacattaatatgaattaaccataggtgtatgtatttcccctcccagctgaatctccctctcacctctggcccattcccacccctctaggttattacagagttcccattcgaattcgaattccttgagttgatgaccattgtgatagcttatgttgaatggtgtcagatttttggcttccagagcagacagtttcgatccggggccagagacaaaatttgatcactcagagcttttgcgtggcagttttattacagtgaaaaggggacagagaaagcttctgacatagacatcagaaggggaacaggagactgtcccactagctagtctcatcaagaccttatataatttttcagttggttactaacaatggaaaggtcttatcaaaccctctccctaaatcctaaaataacaggattagtcagaaggttcttaagaaggagaaccatgtcctcaagcaacatccattgtttttctataatcattagtggacttccctgatggctcagaaggtaaagcctctgtctacaatgcgagagacctgggttcgatccctgggttgggaagattccctgagaaggaaatagcaacccactccggtactcttgccttgaaaatcccttgaaaatcccgtggatggaggagcttggtgcaggctactgtccatggggttgcaaagagtcgggaatgactgcgcgacttcactttcactttcactttctttcaatcattagtataaagtttaaattcatcttgagcaagatgaattgttttgttgtttaatcatttagttaagttcagttcagtcactcagtcatgtccgactctttgcgaccccatgaatcacagcacgccaggcctccctgtccatctcctcccggagttcacccaacgtcacgtccatcgagtcggtgatgccatccagccatctcatcctctgtcgtccccttttcctcctgcccccaatccctcccagcatcagggtcttttccagtgagtcaactcttcacatgaggtggccaaagtactggagtttcagttttagcatcattccttccaaacaacaccccagggcttatctcctttagaatggactggttggatctccctgcagtccaaggggctcgcaagaatcttctccaacaccacagttcaaaagcatcaattcttcggcactcagctttcttcacagtccaactctcacatccgtacatgaccactggaaaaaccatagccttgactagacggacctttgttggaacgtaatgtctctgcttttcaatatgctatctaggttggtcatagctttccttccaaggagtaagcatcatttaatcattagctctgggcttaaagaaagttaatcttcagactgttgtcaaaacaactcaaagtttaaggaaaaatttcttatgtgactaagacaatgtaatgcaaaaaaaaatagtttgtccttttctcctccttgacaactccagaccccttgatggccctggactccttatcaatctacctaagagttaactctctcaactggaagatcctatttccctacattcttctattgtttaacaattcaaaaaatttaaagtcatttttgagttacaatattgttcctgttgtttttatgttcttgttttttaggccacaagacatgtcagatcttagctccctgaacagaatagaactctcaccccctgcattagaccaagcttaaacactagactgtcatggaagtcccagtatttccccagattttatcttctgaacttggcgaaataatgtccaccctcaatgatatgacttcttcctaataaagaactttcagctcaatttcctgattcattttactcattttttttttttttctttccgctaaggatatgggaaagtccctgaggtctacagatagaaaggctttgtgtcttatacagagagaacaagggaaatgagaaaagtaaggggaccacacagtcaataacggatgacattcctgtctcaaagcataagaactataagaaggtttaaaatatagtgaggatactagcattacaggggctcatgggaaacttcaaatttagacgcaaacttcagggtaacactctgatcatatccactaagtatcaagaaataatgtgctcagagacaaaatgattgctcgagaaactgttattaatgtttccttttttcctacttcccttgcctgcacattttaggcaacaatgctatgttaaaccaggtgttcctattctaatgtataatctccctctcccatttgccccaaaacacctccaaaattaaccccaacctccgatttcaggtaaagctcagtgctcacataacacttgcttgaatcttttgaccttggcatcagtgaaatttccagtattgcctaaggaaattcttccaaactctcccactccaaacttttcacatttcttcctgtctagcatacttcctgtctagcacaccctgtgctgtgtataagtgtttctgccttttgtactatcctacataattaccatgtcttacatagcagatcctccatagacagtaagtgtatgtttactaagtgtagtaatcaatgagtgaacaattatttcttgtcccaaaactcctgggagtagaggacaatctgaaaaatatgctattgcatagtgtttctttggtgttagtgctacaatatacaatgaggaagcacacaggaaagtaattcactgattagatatttgctttctgattatagtagacttaaaaatttttgaactctatttaatatggagtatatccaattaaaacagttgtgatagttcaaggctcagcaagtgctaaccattaatcatgttccattctttcccaaactccctcgcatccaggctggcacatatcattgattgacttccctgtgctattcagtaagaccttgttggttatccattttaaagatagcagtgtgtacttgtccatcccaaacttcctaactaactatcccttgtgaaattatttttgtcatatgtaaaattggaaggacgtcaggaaccttctaacttgcatgcatttctttaaatctatatcatgctccagttgattaaatttagacttgactacctgaaacagatcatgttgcttccttctataaaatgtgcattcagaataattttatcttactgccaggttattggcagtgcataaagtttactgtttcaccactgtattttgaactgtctgaaggcaaatgagcttttaaatatttatttgtttcttctcatgtttttatacatagtaggcaatgagaatcctttacaccccaacactatgtttaataaccacagggtcacatcccagaatcagagacatctcaggaaaatcttagcaagtttcctttcatacacctgagcacagaaaagtcatcataagaattagacattttcaaaagattttaaagagggtaagagagactgtgtgtttcacaatttggaatgagaaagcaaagtacccttcttccttctaaatgttcataaaaaaatgaaagaatatctgaggaacttactgtactgtcttttgttccttcacctttggaactggccaatgaatctagataaaaagttgggactgaaaattccaatgctggagaattcaagagaacatcactggggtgggatttggatcaagttcaaacagttgctgtattgttggtgttggtttctctgttaggagtgagggagtcaaaggatggaaagagacttggcagcttgttatatgttttgattctttgttaaagcggagcttctgtaaaagtctaaaagtaaaattaattccttaatatttcattttatttgataatttacccaatagttacttctagttccatcctatcatgttgcttcatagatcatgtttagtatgtgttaatgcaaatggtatatatatatatagaagaaagaaagatggaaagagaccagaggcaaggagagaaggacaacgacagggaggcagacacagagagtcaaagagagagaaagggggaggggggagggggaaggagaggagagagagagagaagtaagtaatgttgtgcattgatttccataatcattttaaaatcagccagttgaatgtcatgggaatttagaatgctattctagattttccatctcttaagcctcagagcagtgctctgaaagttggctttgggagttaatgccttatatatttgtcaagggttcccagtctgaatccatgattctgcctttccagacatacagcttcaaatttcacttgagaaagactgcccctttgttcttgataaatcaatatcatagcccacttttgaggtaaataaaggctaattctgaatgaattaaaacctgaggccagtggaggaagtattaatatacaattgataaactgagacattaactgtcctgtaataattaggatatattttattaaagttattactatattgacttgactgtttttagaaaaaaaaatatgaccttttaaaacatgagttaaatcgcaccatttctaaactattctgtttatgacctctgtttctatgagttctgctcttcaagaagaatttacttcatttcattttttgttttattttattctgctttatttttgaaacaatgctaaaactgaggtaactaagatattgtacatctggggaagaagattaagtgataaccactttatcttgtaataaactttaatggaaaataatcaaataaatagctactatacccagaaaaatacagatgagtttggtgaaatattatacttgggtaattaaaaattgaacatcaaagcatgtacaaaagaattctagctgagtgaaatttttgaaccaaatgatgaaaccacacagctctggatgaatctcatgattctcttgaagaaagtcaatattcacccacttaataatactcagattaggcccatcctggtatagaacattaggaattaggtcaccttgccagactgcaataaaggggaaaaaaaaacaaaacaaaactgtctatttctctctgtgtggacttgtatgagtgtgtgcttgtgtgtgtgtgtgttcagtgcccagtgggtgcgaatcaaagtagagcagacatgctgtggatcccttcacagagtacaagaaagctcatgatggtgaagatggtagtgggcctaagaagcattccaaaacttttttagtggtgacctggatttgcactttaggctctgaacataaccctacttatataactattcacgctttcttttttgtgcctgaacttttgattgtcttataacaacttgcagccttatccctgcagagttatggtaatagaaagaaaaacctgtttcactctttaatcataccaagtagactgaagcgacttaggcagtagcagcagcagcaatggtgaaagacaacaactcttgccctgagacttgcatgatggtggctgcagcaggtcagtacaaaagaggggctcatagaccattcatggaacaagaaagacataaaaaagaagacacagtagaattatttgtaaatatatgtatgtgtgtgtgtgtatgtgtatacacaccaatatatactgtaatactactcaggtataaaaaagaatgacatttttccagttgcaacaacttggatgaacctgtgggatattatgcttggtgaaatgtcagacaaagaaagacaaaatcttgtttattgtgacttatatgtggaacataaaaagcaaaacaaacaaattaatataacaaagcagaaacaaactcacagatatagagaacaaactactggttaccatggggagagaggggaggggagggccaggatagggaaaggaaattaaaagatacaaattactgtgtacaaaatagattagcaacaaggatatattgtacagaacagaaaacatagtcaataatttattgcaactttaaatggtgcatagtccttaaaaatactgaatcactatgttgtatatctgaaactaatataatattatagatcaaatattttaaaataattaattcgaatcaaatgaattagggaaatcatttctgaaaacagaaaaggataggggaagtctttaagtacatgctaatttctgtcagtcaagagggcaaaattgtagccctggcaatttggctcctttacaagaaaatatagaaatagactatactattagttagtaagttctgtgagttttccataacacagagttcctgaaaagatatgtccaaagttttccagaagaagtctttaactcacatatgcctgacctctgatatcagagatataaaataggtcttttaaagagtacaaatataaaacaatgataaagctaatggagatttcagtttaatcgattgaaaatcaaaactcaagatcaaatcaaatgtagctttttgtttttaacatttaaaatatttaaagatttcttttcccagaagtcaacatgtggaaatttttttctaagtatagaccaatagttagagcttctttctttgaaggttgcagaaaagagtgaatataaattgaaaatgtttaaactctatattttttcccctataatcttgtgggcatataatcctaccctaacttccagagagccaagcaagagccaacacttttctacatttctgaaaatatcaggatgaatttgttcacaaattacttgaatgggaatgggccagatcaaaggggtaaggaatcttcatttacctagcaatgatggaaaaaaaatattttggaggctggagtaattaaaactggacatttatgaatttctttgtaacaactagaaatgcagaagtcctttcattctagttcccctaaaaatatattagttggagttacagagacatagagctgaaagaatgagagatttttaaaaatacgttttgccgggtgatgggcaaaaggtgagagaaaggtgattctagacatcaagttgtctgaatcaggagaaggttctaaggtgagagaagggaagtaaatatatatgctcctctccttactaaaaggaagtggaaattcaagaagaagggaagatgcagaaagtagagatcatagaaagtagggggacctcagggaactctgagatctaatgctgagctcagaaatgctttctgcttttgctttttcattaaatcttctagtagtaagaagcagacctctacacttcagaagttcttcttccctaagaaggttaagaggatttattaccacgggttggaggaggtgcaaatagagaagaaatatttgaaaagctgtagataaagaaggatacattttagtaaaagaggtataaacaaagtatatagagaaaacagaattaaagatgaactgaaatagaagaaatttttatgcacactgtactcttagaggtttaggggagaaaaccaaataatctgattcaaactggaatgcctttccacaattttatccctccttttaaatcacagagaagtttctctgttcttgaagacactttcccaggtcagtttatgaggaaacaatttatatgcctcgatttagtctttgaacacattgcctatctcagggttattgaaactttgggagtttgattgattttttactttgtatttcttactacatggaggggtctctatgttttcatctatgggttaatggagtagctatttcataggaattcttgatccatacttattctatttttctatcattatttatttggtcataaattaaattaagaaaaaatgaatgagtagattagcaaatgaatatttgttttcataccagaacaacttaatcccaaacaagcaaacaaaagagatgcatatttagaacagggacagaggttttatccatgctgtccttgtaattattttgtgtattctgaaggcacaggagatgatccatccacatcgtcttaagctgaatcatagtggacaaatcctttccactttctggagcccggattcttcatttgtataataagaaaattgaggaggtggtttccaagagattacttcgttgctgattctaaaatctctgtaagcaaacttgctaaggaagatgattttagtagcagtttgtattgctggaatgactgagacctagagatgcccagaaagagggctgaaggtttaaagtcagtgccaggaagactaaggagaggtatgactgtcatcattcaagcctcaccctgtggaaccacaccttggcctgagccaatctgctcacagaagcagggaggtcaggaggcagggctaagcataaaaggaagagctgggccagctgctgcttacacttgcttctgacacaaccgtgttcactagcaactacacaaacagacaccatgctgagcgctgaggagaaggctgccgtcacctccctatttgccaaggtgaaagtggatgaagttggtggtgaggccctgggcaggtaggtatcctgcttacaagacaggtttgctgctgctgctgctaagtcgcttcagtcgtgtccgactctgtgcgaccccatagacggcagcccatcaggcttcaccgtccctgggattctccaggcaagaacattggagtgggttgccatttccttctccaatgcatgaaagtgaaaagtgaaagtgaagtcgctcagtcatgtccccatggactgcagcgatcccatggactgcgccttccaggctcctccgtccatgggattttccaggcaagagtaccggagtggggtgccattgccttctccgacaagacaggtttaaggagagtgaaatgcacctgggcgtgtgaggacaaagccgtccctgagattctgaaagctgctgacttcctctgaccttgtgctgttttctccccctaggctgctggttgtctacccctggactcagaggttctttgagtcctttggggacttgtcctctgccgatgccattttgggaaaccctaaggtgaaggcccatggcaagaaggtgctggactccttctgtgagggcctgaagcaacttgatgacctcaagggtgcctttgcttcgctgagtgagctgcactgtgataagctgcacgtggatcctgagaacttcagggtgagtttgtggaatcctcaatattctctttttatggtcaagcttgtgtcatggggagagggctgactggcaggacgtagtttagaatggagaagatgtattctggttaaagtgctaaggactcctcagaaccatttagattcttttaatctctttgctcacaataatcatttcttctgattcattcttgttctctgttgtctgcaatgtcttctctttttaattatactctttgagtgttcaatttgaaaaaaagatttcttcatctactttaaacataatatctaatattttccccttatctgttcctttcaagggataaaatgttgtattgcttttcgaagtgattcaaaataataaaaatgataacaagttctggattaagatagaaagaaagaaacatttctaaatataaattcagcctgctatgggtaccttcatatcagcagtagcatctatacttcagtcatctttgtgctaataccctaggggcacagcttgggatgaatctgaagtactctgaatctaacctgggcacctgcactaaccctgcccttgcttaatgtcttttccacacagctcctgggcaacgtgctggtggttgtgctggctcgccgctttggcagtgaattctccccggagctgcaggctagctttcagaaggtggtgactggtgtggccaatgccctggcccacagatatcactaagctccccttcctgattttcaggaaaggtcttttcatcctcagagcccaaaaactgaatatggaaaaattatgaagcgttttgtgcatcttgcctctgcctaataaagacatttattttcattgcactggtgtatttaaattatttcactgtctcttactcagatgggcacacgggagggcaaagcactgaagacataaagaaatgaaggcctaagttgagactttgagaaaatacatcagtatcttggaccccatgacaggagtggttgtacacagctgatgttattggaaaacaggctctgctccttactcttactttcctttaaagaattccattctccccaggttgctttaacatctacacacggaagtgactagaggtctgccctctttatctggtctctgatggcttctctgcctttgtagtcattagcatactgttaccagaagcaacatatgccaatttaatcctctccttcaataaccgcttatccgtatacttgattctggaaatgatgtatgaatttttagtaatccttgtttcctcttgcatcctaattctgaatctctgttctgttcagtccagtggctcagttgtgtacaattctttgcaaccccatggactgcagcatgccaggcttccctgtccatcaccaactcctggagcttgctcaaattcatgcccattgagttggtgatgccattcaaccatctcatcctctgttatccccttctcttgccttcaatctttcccagcatcagggtcttttccaatgagtcagttcttcacatcacgtgtccaaagtattgaagcttcacgttcggtatcagtccttccaatgaatattcaggactaatatccttcaggtttgattggtttgatctccttgcagtccaagggactctcaggagtcttctccaacatcacagttcaaaagcatcaattcttcagcggtcacctttcttcattgtccaactctcacatccacacatgactactggaaaaaccatagccttgactagatggacatttgttgataaagtagtgtctctgctttttaatatgctgtctagatttgtgatagcttttcttccaagaagcaagcatctttgaatttcatggctgcagtcaccatcttcagtgactttggagccccccaaaataaagtgtctcactgtttccattgcttccccatatatttgccatcaaatgatggtaccagatgccatggtcttggttttttgaatgctgacttttacgccagctctttcactctcctctttcactttcaccaagaggctctttagctcctctttgctttcttccataaggggggtgaccactgcatatctgagcttattgatatttctccaggcaatcttgattccagcttgtgcttcatccagcctgccattttgcataatgtacttggcatagaggttaaataagcagggtgacaatacacagccttgacgcattcctttccctattctgaatctagttggggaggttctgaaagtaacgcatctaacaagtgactatctaataacgcatctaataaatgaatatacaaggcttttatgagatctgaggatgaagcccttggtatgaaatggtggtaaagataagtagtatagacaattgtccagaagacaagaagacaaaagtcagaaaagaaaaggaggtcaagtcactgatgaataatgtgtttgtgtattctgagaatttcaggtaaacaggaaagataactgaagcaaaactgcctgagatagaatagaatagattataaattttggtgaacacagtggagcgtattcttaaagttaaaaattcacagacaagaggatacaattcagccattactcacaagatgtttgcacagccaattgacaagtaaagtaagatagtttaacagtttagaacatcattttgaaatcagacagcaaggattgaaaaaacattactacttattagctctgaccttgtgaaaattctgctgtcaatttggtatataaatatgtcgtctttaagatttgcaggaaaatgaaaaaaaataaaagtctcaacacaattccatactttgacctttaacaaaagttaatgtcaggtgagaatttacaaagagtaagttgactcatttaacatctcttcctaaatgcaaaagatctcaggccggggtggcaatattgaagagtagggaatttgggggccttttttttttttttggatcttaacaacaggcaaccatgctagcagcgtctatgtctagaagaaagttgaattactatgtacatctggaatcaacaaattaaaagtctcttatttttaagacaaatgttttagggggtctaatcagagatgaaaatgtgatggtaggaagcagcttgagaacagtccagttgaaataacacagggcacaaaaaagagacaaagatatcaaataatataaaaatgttaatcagttggctgatgcatagatcatcaaatgccctcttatattttgctgttgctgagtattttcaaggacggctaacaagagcatggatgctcatttatgttaagtctaatgagatcatacaaatcctgatgggaacttctcaaacacccagatgaccaccctgcaacataaattttttttttgctgtctaagaaatggatgtcatttttcaatactcattcattataagaatattatttatagataccttatataatacatagtaagtaaaataatatgctgtgcttagtcactcagtcatgtctgagtctttgtccccatggactgtggcacctctgtccatggcgattctccaggcaagaatactggagtggcttgccatgtccttctgcaggggatcttcccaacccaggaatggaaccgagtctcctggataaaggtggattctttaccagtggaactaccataaatatatgtaaatatacatataggtatgtaaacatacatatgtatagatatgtaaacatacataaagatagtacataataaaggggaacattcaagaaatgaatagttatagtcattcacataaactgtgatttgaagactattctagatttgtggcaaataagctgtctctgatgcaaatgtatttacaacccctgaacatctagttctctagactaggcttataaagtgttcttatctttcagtcttttcaaaactcttaaatacttgacctgacctatatttactcaattcagaacaaatctgccttatgcattttattcatctttgaggcttccctggtggctcagatggcaaagaatctgcctgcaaagtgggagacctgggttcgacccctggaaaagatgtgggaagagcccctggagttgagcatggaaatcctgtacaatattcttgcctggagaatccccatggacagagaagctggcgggctatagtccatgtggtcacaagagtttgacaggaccgacctactaagcacattatgctttgaagaaagggaaaatttcacagcctgctctgccccctgctggcatcatgtgaagaatcatctatttaagagtgacactatcatgaatttttcgaggcaggtccagtgtgggtgccattgaataattatggtgaagtacgaacaaaaatctggattttcctggaatacagagttgttcttattcaatgtaattataaagatactgatttttagacttaacactctccctctgcctttggctttccaggtggcgcaagtggtaaagaatccttctgccaatgcaggagatgcaaggacctaggttccatctctgggttgggaagatcccctggggtaggaaatggcaacccacttaaatattctggcctgaaaaaaaccatcgacagaggagcctggcaggctacagtccatggggttgcaaaaagtcagatacaactgagctactaacacttcttatgcctttaggttagtcaaggattgttggtcattacctattccaaaaaaagtcatgaagtaatcacatagggaatcatttccaaaggacagaaaaaaagtaccataggtgtctgaacacataagtcagtttaaactgtagaatataagaaaagactgccctgaaaaaaggtttctgtttaatcagtcccagatatgcttccaaatcagcattccagacaaagtgctcagaggaagaccatgatcctatcgttacagtggatggaaactcacctatgcttgtcctctccagttgtttattttatgtcagacagtattctggttgcccaattcaggaagaagtattctgcaccctgactttgtggtggtcacataacacagcatgatgtttggggaaataatgggtctctttttttcttcttggtacaggtgaagcatctgacatctctgaataatttccctgtggaaccttcatttatcattctcattaacactgtactttcctggctaccatcctacatctcaaacagaaaaaaaaaaaaaattccaacatttatacttttctgttgctgtaatgttgatattctctttggcagtaccattaacattcatggcttctctgaagaagcttgaagaagagtatgttctttcagtgcaggcctacattcacagaatagttgcatcttttggcatcaatcccagatcagatcagatcagatcagtcgctcagtcgtgtccgactctttgcgaccccatgaattgcagcacgccaggcctccctgtccatcaccaactcccggagttcactcagactcacgtccatcgagtcagtgatgccatccagccatctcatcctctgtcgtccccttctcctcctgcccccaatctctcccagcatcagagtcttttccaatgagtcaactcttcgcatgaggtggccaaagtactggagtttcagctttagcatcattccttccaaagaaatcccagggctgatctccttcagaatggactggttggatctccttgcagtccaagggactctcaagagtcttctccaacaccacagttcaaaagcatcaatttttcggtgctcagccttcttcacagtccaactctcacatccatacatgaccacaggaaaaaacatagccgtgactagacgaacctttgttggcaaagtaatgtctctgcttttcaatatgctatctaggttggtcataactttccttccaaggagtaagcatcttttaatttcatggctgcagtcaccatctgtagtgattttggagcccagaaaaataaagtctgccactgtttccactgtttccccatctatttcccatgaagtgatggaaccggatgccatgatcttcattttctgaatgttgagctttaagtcaactttttcactctccactttcactttcatcaagaggcttttgagttcctcttcattttctgtcataagggtggtgtcatctgcatatctgaggttattgatatttgtcccggcaatcttgattccggcctgtgtttcttccagtccagcgtttctcatgatgtactctgcacagaagttaaataaacagggtgacaatatacagccttgacgaactccttttcctatttggaaccagtctgttgttccatgtccagttctaactgttgcttcctgacctgcatacaaatttctcaagaggcagatcaggtggtatggtattcccatctctttcagaattttccacagttgattgtgatccacacagtcaaagggtttggcatagtcaataaagcagaaatagatgcttttctggaactctcttgctttttccatgatccagcagatgttggcaatttgattctctggttcctctgcctttctaaatccagcttgaacatctagaagttcacggttcacatattcgtgaagcctggcttggagaattttgagcattactttactagcgtgggagatgagtgcaattgtgcggtagtgtgagcattctttgcattgcctttctttggagttggaatgaaaactgaccttttccagtcctgtggccactgctgagttttccaaatttgctggcatattgagtgcagcactttcacagcatcatctttcaggatttggaatagctcaactggaattcgaaaactgaccttttccagtcctgtggccactgctgagttttccaaatttgctggcatat

SEQ ID NO: 14 - DNA, 95 bp, Ovis sp.
aattcgagaaaataatgaatgtcaaaggaaaagtgattctgtcaatgctggttgtctcaactgtcattgttgtgttttgggaatatatccacagg

SEQ ID NO: 15 - DNA, 2479 bp, Ovis sp.
gagttctgcttgcctgtgagtctccattcaggtgttcacaggctccttgaaattcatgcatccaagagggaactcatcattattttcctccaagcctattcctcctccaaccaatctgactgaattctacaacctacctgcttaagcagaaaccttatttcttccttcctccctaatggctaaattaccagccaaatcctatcaattctctctctaaagtatgttttgaatgaaatcaaccattgagccagcccatctccccttttttaaagaatttcatgtatttaattttggttgccctggctctttgttgctaggcaagggctttctctagttgtggtgggcagacttctcattgcgatggcttcttttgttgcggatcacaggctctgagcatgtggcccttcagtggctgtagcacataggctcaacagtagttgtggcttgagggcttcagagcgcctggtatgtgggatcatcccagactagggattgaacccttgtcccctgcatcggcaggcagattcttaaccactggaccaccagggacgtctccaccccatcacccctgttgcaattagaatggtctagcttgtcgttttatccgttccccacacccagtgcccagatgggtctctctgaatgaagatgggtcgggtgactctccagcttcagatgcttttgtgggtccccaccctctttccatgcccttccagcctctgactgcccctgcccgccctctgactcctgccatcaccaccaccacccgcctcttcactcaggtgagacagctgcactattttttctcgtccacgtttttgcttttgtttcccaaacacaggaagcttttctctaatcaacctggtatagatgctatttctgaaatagtctcatcccttctcatgggactttcttttccatcctatcttcaccccatgggaagctttaaaatcatctgcccagtaagcccttccccaaccacccaaatcaaagtacttcccaagctgtagttctctaaccagcaccccttccccctttgatcacaatttcatgcagcaatgaagaaatatttatgtacctactacgcaccagctgtgtgggtatgggagggaaaggcctacctgggaaatggttgggtctcaattgtaaaagaccagcatgctttctgctctgaacggcggagcacgtagttaggatatcagaggcagcagagccattgaactgatcattccccaaggtcacatgcagagagttagtgctatcgagtcagaggaggccagcaaccatgggaaccattagctcaaccctcactctagaaaaatggtacagatgaacttgtttgcaaagtagaaatagtcacagatgtagagaacaaatttaaggttatcaagtgggggaggggcgagatggggtgaattgagggattggtattcacatactgatcctgctgtgtattaaatagataactaatgagaacctgatgactaatgagaaccttctggaacacaaggaactgtactcagtgctctgtggtgacctaactgggaaggcggagaaggcaatggcatcccactccagtaaccttgcctggaaaatcccgtggatggaggagcctggtaggctgcagtccatggggtcgctaagagtcagacacgactgatcaacttcactttcacttttccctttcatgcattggagaaggaaatggcaacccactccagtgttcttgcctggagaatcccagggacgggggagcctggtgggctgccatctctggggtcgcacagagtcggacacgactgaagtgacttagcagtagcagcagctaggaaggaaatctaaaaatgaggggatatgtgtatatgtatagctgatttactttactgtatagtgaaaactaatacaacattttagagtaactgtactgtaataaaaatgaataaaccctctcccatgtctatggtggatcttgtgatcagtcaggatactttccctctgagcctcaaagtggccttagcatctttcgcaactcaggcagccccatcagttcattgaattggctatttgccgttgcagacccatgcaatttaaggtagaaccttctccttgcacgtgtgcctgctcagttgcttcagtcgtgtccaactctttgcaacgctatggactgttgcccaccggacacttctgtccatggaattctccaggcaagagtactggagtgggttgccgtgccctccttcagggggtcttctccatccagggatcgaacttgcatctcctgcattgcaggcggattcttcactgctgagcccctggggaagcccagaacatgtgccttagcacttgttaaatattcatcaccttttccttttagaaaggacataggtagaaataattattgaaaaaaatcatatcccactcttgatatatttaatctattttcccccctcttcttttcttttcccaggagaaaataatg

SEQ ID NO: 16 - DNA, 1096 bp, Ovis sp.
gttttgggaatatatccacaggtaattatggaacatgataaagtgatgttaatgaacgtctccatcagccaagtcaccaggttgaattgaaattaggacttcttccttcctgtttccctgagccctagactttaatgactctctttaaaatcattagagcttcaagcactttctaaggaattaattatctttcttcctgccatgttcctaatatctcaatttttatatagcttgttctgggcaggctagagccagaaacagtaaggtcttattaagaccaaaatgtcatattaaaatatgaccagatatggaaattgcattaagaaatttcagacaggaattccacgagaaattcaccctgatttttgcagtcctaaaatatttgcaaagttcaaaggaacaactcaaagttgttgacttttgctgcaaaatacactttgagtcgctggtgattcatttgtgcctggctaaacttttgagtgctttgtctgttttttttttttttttaactctggaaaacaaaatgaatgaaatatttctgagttttcaaattcatcagtggattcacctcaaatatttgagctgcttcatgtgttttgagaaaatataatgccttggaggttccagctggagaggcttctgacagaaagaagtctccccaggcagctacaaaaatgcacaatggttttgactttaagaggcatccgtacagctcagcctttctttcgaaaaggccaccttacacctagaggtggatgaacctagagcctgttatacatagtgaaataagtcagaaagagagaaacaagtgttatatattaacgcttttatatggaatctggaaagatggtgcagatgaaactatctgcagagcagcaaaggagatgcagactcaaagaacaggcttgtggacacagtgagggaaggagagggtgcaatgaactgagagaacagcattgaaaaatatatattgccgtatgtaaatagagagccagtgggaatttgctgggtgctgcagggagatcaaatccaatgctctgtgacaatcttagaggggtgtgatgggatgggtggtgggagggaggtccagaggggg

SEQ ID NO: 17 - DNA, 815 bp, Ovis sp.
tgcagactcaaagaacaggcttgtggacacagtgagggaaggagagggtgcaatgaactgagagaacagcattgaaaaatatatattgccgtatgtaaaatagagagccagtgggaatttgctgggtgctgcagggagatcaaatccaatgctctgtgacaatttagaggggtgtgatgggatgggtggtgggagggaggttcaagagggakgggacatatgtatacgtatgactgattcatgtttatgtatggcagaaaccaacacaatattgttattatcctccaattagaaataaattcaaataaataaaaaaggccaccgtacaacaactctgggccaggagcactgtcttgtcactattgatatcttcatggtcttagtaggttcccaagttcaaaatgatggcctggataaacattcagttcagttcagtcgttcagtcgtgtcckactctttgcgaccccatgtaatcgcagcacgccaggcctccctgtctatcaccaactcctagagttcactcaaactcgtgtccattgagttggtgttgccatccagccatctcatcctctgtcgtccccttctcctcttgcccccaatccctcccagcttcatgagtcttttccatatgagtcaacttcttctcatgaggtggccttagtactggagtttcagctttagcatcattccttccaaagaacacccagggctgatctcctttagaatggactggttggatctccttgcagtccaagggactctcaagagtcttctccaacaccacagttcaaaagcatcgattcttcggggctcag

SEQ ID NO: 18 - DNA, 11034 bp, Ovis sp.
gagttctgcttgcctgtgagtctccattcaggtgttcacaggctccttgaaattcatgcatccaagagggaactcatcattattttcctccaagcctattcctcctccaaccaatctgactgaattctacaacctacctgcttaagcagaaaccttatttcttccttcctccctaatggctaaattaccagccaaatcctatcaattctctctctaaagtatgttttgaatgaaatcaaccattgagccagcccatctccccttttttaaagaatttcatgtatttaattttggttgccctggctctttgttgctaggcaagggctttctctagttgtggtgggcagacttctcattgcgatggcttcttttgttgcggatcacaggctctgagcatgtggcccttcagtggctgtagcacataggctcaacagtagttgtggcttgagggcttcagagcgcctggtatgtgggatcatcccagactagggattgaacccttgtcccctgcatcggcaggcagattcttaaccactggaccaccagggacgtctccaccccatcacccctgttgcaattagaatggtctagcttgtcgttttatccgttccccacacccagtgcccagatgggtctctctgaatgaagatgggtcgggtgactctccagcttcagatgcttttgtgggtccccaccctctttccatgcccttccagcctctgactgcccctgcccgccctctgactcctgccatcaccaccaccacccgcctcttcactcaggtgagacagctgcactattttttctcgtccacgtttttgcttttgtttcccaaacacaggaagcttttctctaatcaacctggtatagatgctatttctgaaatagtctcatcccttctcatgggactttcttttccatcctatcttcaccccatgggaagctttaaaatcatctgcccagtaagcccttccccaaccacccaaatcaaagtacttcccaagctgtagttctctaaccagcaccccttccccctttgatcacaatttcatgcagcaatgaagaaatatttatgtacctactacgcaccagctgtgtgggtatgggagggaaaggcctacctgggaaatggttgggtctcaattgtaaaagaccagcatgctttctgctctgaacggcggagcacgtagttaggatatcagaggcagcagagccattgaactgatcattccccaaggtcacatgcagagagttagtgctatcgagtcagaggaggccagcaaccatgggaaccattagctcaaccctcactctagaaaaatggtacagatgaacttgtttgcaaagtagaaatagtcacagatgtagagaacaaatttaaggttatcaagtgggggaggggcgagatggggtgaattgagggattggtattcacatactgatcctgctgtgtattaaatagataactaatgagaacctgatgactaatgagaaccttctggaacacaaggaactgtactcagtgctctgtggtgacctaactgggaaggcggagaaggcaatggcatcccactccagtaaccttgcctggaaaatcccgtggatggaggagcctggtaggctgcagtccatggggtcgctaagagtcagacacgactgatcaacttcactttcacttttccctttcatgcattggagaaggaaatggcaacccactccagtgttcttgcctggagaatcccagggacgggggagcctggtgggctgccatctctggggtcgcacagagtcggacacgactgaagtgacttagcagtagcagcagctaggaaggaaatctaaaaatgaggggatatgtgtatatgtatagctgatttactttactgtatagtgaaaactaatacaacattttagagtaactgtactgtaataaaaatgaataaaccctctcccatgtctatggtggatcttgtgatcagtcaggatactttccctctgagcctcaaagtggccttagcatctttcgcaactcaggcagccccatcagttcattgaattggctatttgccgttgcagacccatgcaatttaaggtagaaccttctccttgcacgtgtgcctgctcagttgcttcagtcgtgtccaactctttgcaacgctatggactgttgcccaccggacacttctgtccatggaattctccaggcaagagtactggagtgggttgccgtgccctccttcagggggtcttctccatccagggatcgaacttgcatctcctgcattgcaggcggattcttcactgctgagcccctggggaagcccagaacatgtgccttagcacttgttaaatattcatcaccttttccttttagaaaggacataggtagaaataattattgaaaaaaatcatatcccactcttgatatatttaatctattttcccccctcttcttttcttttcccaggagaaaataatgntrnnbndarystartcdnndrndgrstnsncndcatdasngrsdnntrnbndarygttttgggaatatatccacaggtaattatggaacatgataaagtgatgttaatgaacgtctccatcagccaagtcaccaggttgaattgaaattaggacttcttccttcctgtttccctgagccctagactttaatgactctctttaaaatcattagagcttcaagcactttctaaggaattaattatctttcttcctgccatgttcctaatatctcaatttttatatagcttgttctgggcaggctagagccagaaacagtaaggtcttattaagaccaaaatgtcatattaaaatatgaccagatatggaaattgcattaagaaatttcagacaggaattccacgagaaattcaccctgatttttgcagtcctaaaatatttgcaaagttcaaaggaacaactcaaagttgttgacttttgctgcaaaatacactttgagtcgctggtgattcatttgtgcctggctaaacttttgagtgctttgtctgttttttttttttttttaactctggaaaacaaaatgaatgaaatatttctgagttttcaaattcatcagtggattcacctcaaatatttgagctgcttcatgtgttttgagaaaatataatgccttggaggttccagctggagaggcttctgacagaaagaagtctccccaggcagctacaaaaatgcacaatggttttgactttaagaggcatccgtacagctcagcctttctttcgaaaaggccaccttacacctagaggtggatgaacctagagcctgttatacatagtgaaataagtcagaaagagagaaacaagtgttatatattaacgcttttatatggaatctggaaagatggtgcagatgaaactatctgcagagcagcaaaggagatgcagactcaaagaacaggcttgtggacacagtgagggaaggagagggtgcaatgaactgagagaacagcattgaaaaatatatattgccgtatgtaaatagagagccagtgggaatttgctgggtgctgcagggagatcaaatccaatgctctgtgacaatcttagaggggtgtgatgggatgggtggtgggagggaggtccagaggggggrstnsncndcatdasngrsdtgcagactcaaagaacaggcttgtggacacagtgagggaaggagagggtgcaatgaactgagagaacagcattgaaaaatatatattgccgtatgtaaaatagagagccagtgggaatttgctgggtgctgcagggagatcaaatccaatgctctgtgacaatttagaggggtgtgatgggatgggtggtgggagggaggttcaagagggakgggacatatgtatacgtatgactgattcatgtttatgtatggcagaaaccaacacaatattgttattatcctccaattagaaataaattcaaataaataaaaaaggccaccgtacaacaactctgggccaggagcactgtcttgtcactattgatatcttcatggtcttagtaggttcccaagttcaaaatgatggcctggataaacattcagttcagttcagtcgttcagtcgtgtcckactctttgcgaccccatgtaatcgcagcacgccaggcctccctgtctatcaccaactcctagagttcactcaaactcgtgtccattgagttggtgttgccatccagccatctcatcctctgtcgtccccttctcctcttgcccccaatccctcccagcttcatgagtcttttccatatgagtcaacttcttctcatgaggtggccttagtactggagtttcagctttagcatcattccttccaaagaacacccagggctgatctcctttagaatggactggttggatctccttgcagtccaagggactctcaagagtcttctccaacaccacagttcaaaagcatcgattcttcggggctcaggrstnsncndcatdasngrsdaagagtcttctccaacaccacagttcaaaagcatcgattcttcggggctcagccttcttcatagtccaactctcacatccatatatgaccactggaaaaaccatagccttgacatagatggacctttgttggcaaagtaatgtctctgcttttgaatatgctataaacattagaatgttgctttcagttcttaaattctaagatttagatgggctggtaggactgtcctggctgcttataaaggatctgtaccttcaggtacatcagtggaaggaggaatctctggactctggagctgtttttccccagggtgacctaaccagttctggagtattattgtttcattctgaaaaacttgttttaaaagaaacaggacaagctagagagtgaccatgttaaaggggtctacaaaccaggtctttcaaattgaaggaaaccatgggtacttagtctgaagaaaaggtgactcagtgttggatgcagggaccctggggctcccttttgctgagggcggaggaagtatcctggctggagtctggaactaggctgcctggggctggtcctgtgccatgacttgcctcctctgtgactttgggcaagttacttcaccactctgttccttggttttcaataagtttccctgttgtttaaaatgggcataatggtagtacctgctttgtagggttgttggattaaataagttattgtgtatagagcacttagggcagtgccctcagaaaacgttcgctgccatcaccatcatcaccaccattctcatcatctgctcagaggtaagatttggcaaatagaaggaagagctccagcagttggaagaatcctacagaagagtgagcgtcctttggggtcttgccctgagctgagctacttgtgtaggtaaccttgagagcatttgagctctgaatgcttgattggataaccttggcgttttgaattgcagccagagtcacacttccaaattttgggtacttcacaaaattaaaatatgggagccaaaggcccaaatgtgcatattggcactgggctcccatagrntrncsncrmarkbtkbwthrscttnndcatdasngrsdtcggccgctctagaactagtggatcccccgggctgcaggaattccaaaaatgccagcnattagatagcccgaacgtgtgtcattgtttctcccacacactcaagaccccaactgagaactaaggaccgaagtgactaagcgcggagcttccccacgtcagcctggctgaccactctccccgggcttccgcagctgcctctgcccttctcctcctagaggctttggcccccggagatctggccaaccagaacttcttcttccagaggttggatgggttcctgtttctgcttccatttccttgtttagaatgacatttctccccaaactgcaatcattcttacttaaaagtcggagctgtgataatagtttaaaatataacccattttgttcctttggtgatttttttttttctttatcaaagcgaaagcaaaggtttacttttcctattagcctcctccctgcaacccccctccacgggccttcccatctgtatcttttctgagcgccacatgggaagttgactgctcgcttgtacagagagtggtgaaggcccgggtggatctgagtgaggatctgtgcatcttcactcccaagaccgtccccagcccagagcccagaagccatcgccctcaccctactggtcaagtcctgatgtcaccacctgtgctgatcccaggggcatctgtgatggagaagggtgactctggaattttgacatttttactggaagaatctgtactgttatgcgtctggagcagatgcctagctcacctcatcctttggtatgagaggggatgagccagccaccctgtagactgatttactcctgatgttccacaaagaacagaatgtggcctgcggttccacgatggcttcaccctcgtattcgcagcttcacagaaccaaacagtaaattgtacaccctgagctctcctcttggaggccggaggggactgcttgtactctgcagcaataaatcctcccaagagtggatctggactttaggaggtgtgaagtttggtttgttttcaaataacagtgcccggagccaattgcaatgctttcatctctaaataatgttaaacctcacaagggccccctcccctccccctaggattgtttcccaagcttcagggcacaatagatttttttttcagagccatggcaaagtccctcttcatattgctcagatatattctactgtaaaccccggggtttttccagagaccaatattaaaaccacagtttatttttgtagaaatgaacaggaatccaaactgcaactaaggagcaaaaacaaatgccagttattattactcacaaaatgtcataactggccgtgagtgtgatcagtaaattaaacttagtccacatccgctgagcatgagtctgggggtgatgcaagttgtgttaagccaactcccgctttccttctctatcatccccacgctgtcacgtgataagtgtgtagtttggctcacaaaagacataaacatgttttcggaatgaataagtaaatagctaaaataccacatctattagaaggacctaagtccaaaacactgacaacacaaactgctggcaaaaatatggagcaagcagaactctgattcactgctggcgagaaggcaagatggttcagctgctttggaagaaagctgaacatattcttatcatacagtctagcaattgcatccttggtattcaccaaaaaaagctgaaactttacagccgcacagaaacttacatacagatgtttaaacgcagctttagtcacagtttctaaaacttggacataactaagatgtcctttagtaggtgaatggattaataaactgttgtggcacgtcaggtggtggaatatattatcagtgcaaaaacacaaatctgtcaagccatggaagagatagaggaacttaatgcatattattaagtaaagaaaccatctgaaagattgcctgctgtcagattccagrntrncsncrmarkbtkbwthrscttnndcatdasngrsdtagggaacaaaagctgggtacgatatcatacatagtgaaaaatacttgtcattacatatttgtcgaaacccatagaatacacacaaccaagagtgaactttttttagaaaaggaaatgagatagttaaaataagtttaaatcagagggtgtttgctctggtatttggatgggtcttataatggtggtagctggataatggagtggtgtttggggtaaccactagaaaattcttaaagggaatgtataatgtggaaggatattcatgtcccccataacccattctctgggtcccagagaagaagagagtggtgatgcttagtcttaaggcatccccaaaggacctaagaggcttgtcaccctttcatctgatagggcaggataacattggagcggatgttggggtttctgagagatgcctgagaactagcgccccctgatgttgatcacggggtacaaccttccagttataaaatgagcaagttctggggatctgatgtaaagcattgtggttattgttaataatattgttttctatactcataaagttattcagataataaatctcaaatcttctccccaaaacagaaatgatgattatgtgatgcaatgaaggtgttaactaatgctactgctgctgctgagtcgcttcagtcgtgtgcgactctgtgcgaccccatagacggcagcccaccaggctcccctgtccctgggattctccaggcaagaacactggagtgggttgccatttccttctccaatgcatgaaggtgaaaagagaaagtgaagtcactcagtcgtgtctgactcttagcgaccccatggactgcagcctaccagccttctccgtccatgggattttccaggcaagagtactggagtggggtgccattgccttctccgaactaatgctacagtggtgggrntrncsncrmarmmdatytnndcatdasngrsdtcctcctccaggggctcttcctgaccccaggcgtcaaacccgcatctcctgcgtctcttgcatcacaggcagattctctacccactgagccatctgggaagcccttttataatatatgaatataccaaatcaacacttggcacattttaaacttaaactatgctatatgtcaattatatctcaataaaacaggaccaaaaaaggactagcacactttcacctacccggagtctgaaccaactgataaataagacaaagggataaataaggtagatggtagtgatgatcatcttcactgtgttaaaagctgatattgaagaatatgggttataaaactgaaggccctgggcctcctgatgttgaaccacagaattagaaagacgtaggcacccttcatccatagcatgagaaactgcaggtgtctggatgttggagaagggcctgtatcatttagctacattgcagggccctagatagatagagatctgtttaaacctcgagggccggagggtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgrntrncsncrmarmmdatytnndcatdasngrsdtcttctatcgccttcttgacgagttcttctgaggggatcggcaataaaaagacagaataaaacgcacgggtgttgggtcgtttgttcggatccgtcgacaggcctacctgcggatattaagcttgtcaagatgtcttggcagacaaaagagtataatgtggttagaaataacgtctgactttgtgccagtacatttttgaatttgagagagtattattctggctacttcctcagaaaagtaacacttaattttaacttaaaaaaaaaaaaaaactaacaaaagaccaacacagcaagtacacattatttctccttgtaacattgagccttgtaatacgggagaatgaacctgtggtaatcggatgtaaattcccagtgatttcttacctattctcagttgcgggggaggggaatggatacactatcagttgaaccaaaaaagagaaaaaattatcatagacaaaagccagaaacattctacatgtgccaattaacttactggagagaagtacgcaaagggacgtgtttggcaacaagacaatgattgtgaggggctgtcctcttgatttcagtgtcttcctgtctctgctgggtctgaagcaacacagagttgcttttgcagcaggagaactcttagtaggacatagtctaccttggcgatcctcaaatggtttagtcagtgtcctttccttatcaggatattggcttctgtgccaagactagtcaagattcccagtacatatggtgaagatgttggggaatcacaggcaatctgagtcagtgggtgctcagcacaaaatgaaaacaaagcctccaagtaagttggccatgaaataagcaaagagagagtagaaaggrntrncsncrmarkbtkbwthrscttnndcatdasngrsdttactcacaagtgtgaaacaccatagaaggcatgaatacataataaatgatgcataccttttaaagagagaaagctagggtaagtggggatctactgttcaagtctcagggattcaaacccagcactgtctgactctaaatcctttgttctttccacagctcaagtctttctctttcaaacacacacacacacacacacacacacacacactttaaccattttgtttttgagaccgagggcatcaaggaatcaccaccaagaactctcaattcttgtactagcattaaagaaagagaacttagttgactcaaacttcacagatggatggtaggtctaaactcatcctggagcctggctggaaagcagtcaaacagaacctgagcgatacgaggctcagggaagttcaagggctgagagggtacagaggagagaaggtctgggaggtggaaacagtctcagaggggctgcttctgaattaagcactgagagaagagaaagggatcactaagaagtggaaatggaatgaggtttcggagagaggaacagcaggtacgaagcatggaggactattctgaggcttttagggagaccctggatgaaatctcataggagagcctgtgtgaaaagcagcaaagagcttgaaagggctttggctggttgggagctgcacatttgcccagcatctgtgtctttctttctcgctttatctccttcatagagtctatgtggtttccatggcaactaatgccatagttgggccaagtgacccagactaagctaatcaacattttccctccccccgcccccaggggggcagatgatctaaactagtccaatcaagttgaatctcaggcctttaattaagcggccgccaccgcggtggagctccattcgccctat

SEQ ID NO: 19 - DNA, 1813 bp, Ovis sp.
tgcagactcaaagaacaggcttgtggacacagtgagggaaggagagggtgcaatgaactgagagaacagcattgaaaaatatatattgccgtatgtaaaatagagagccagtgggaatttgctgggtgctgcagggagatcaaatccaatgctctgtgacaatttagaggggtgtgatgggatgggtggtgggagggaggttcaagagggakgggacatatgtatacgtatgactgattcatgtttatgtatggcagaaaccaacacaatattgttattatcctccaattagaaataaattcaaataaataaaaaaggccaccgtacaacaactctgggccaggagcactgtcttgtcactattgatatcttcatggtcttagtaggttcccaagttcaaaatgatggcctggataaacattcagttcagttcagtcgttcagtcgtgtcckactctttgcgaccccatgtaatcgcagcacgccaggcctccctgtctatcaccaactcctagagttcactcaaactcgtgtccattgagttggtgttgccatccagccatctcatcctctgtcgtccccttctcctcttgcccccaatccctcccagcttcatgagtcttttccatatgagtcaacttcttctcatgaggtggccttagtactggagtttcagctttagcatcattccttccaaagaacacccagggctgatctcctttagaatggactggttggatctccttgcagtccaagggactctcaagagtcttctccaacaccacagttcaaaagcatcgattcttcggggctcagccttcttcatagtccaactctcacatccatatatgaccactggaaaaaccatagccttgacatagatggacctttgttggcaaagtaatgtctctgcttttgaatatgctataaacattagaatgttgctttcagttcttaaattctaagatttagatgggctggtaggactgtcctggctgcttataaaggatctgtaccttcaggtacatcagtggaaggaggaatctctggactctggagctgtttttccccagggtgacctaaccagttctggagtattattgtttcattctgaaaaacttgttttaaaagaaacaggacaagctagagagtgaccatgttaaaggggtctacaaaccaggtctttcaaattgaaggaaaccatgggtacttagtctgaagaaaaggtgactcagtgttggatgcagggaccctggggctcccttttgctgagggcggaggaagtatcctggctggagtctggaactaggctgcctggggctggtcctgtgccatgacttgcctcctctgtgactttgggcaagttacttcaccactctgttccttggttttcaataagtttccctgttgtttaaaatgggcataatggtagtacctgctttgtagggttgttggattaaataagttattgtgtatagagcacttagggcagtgccctcagaaaacgttcgctgccatcaccatcatcaccaccattctcatcatctgctcagaggtaagatttggcaaatagaaggaagagctccagcagttggaagaatcctacagaagagtgagcgtcctttggggtcttgccctgagctgagctacttgtgtaggtaaccttgagagcatttgagctctgaatgcttgattggataaccttggcgttttgaattgcagccagagtcacacttccaaattttgggtacttcacaaaattaaaatatgggagccaaaggcccaaatgtgcatattggcactgggctcccata

SEQ ID NO: 20 - DNA, 1379 bp, Ovis sp.
actggcacacccgggggtgcaggtagagctgggctgtgggtttgagacgagacccggggcaccgggagtgggtgggagggcctggttggttctagaacaggaggaccgcagagcttggtggcggaaggacaggaggtgtggacgagtgtctcttgggggctggggtgaggaggaggtgacctggccttgcccagcccccctggaaaaacttagcttcacccctggcagaggggcagtgttgtgaaaacccactctccacccatgtgtgagggagagaggaagttctcagtgggctgatctatttgccaatatctctttgatagcctcaccaaggccacttgcacacagcaggggcccgatggggccaccaagactttgccccagtcactgggcctctccccgcagtggctgaagtgcaggcacatggcgtggcccttgtgtgacaggggttgctctaaatgactgtgcttcagtctccacttcccgccagggaagggcagtctcttgcaaacccaaatggggttggagctgagtccccaccccattcccgccttagcctgggccccagccgctgctcagcctttctgcatttactctttatggttggttggtttgtcttcgctcttacattgcttcctgtgtttgtattggctctgccaagtccctttcaagtgtgctgagtgcacagaacacccccctcccccacccccggctttgtaaggaggaaacgtgatctgagagaggtgacagccagggtccctgtgtttcatccctggctactgtgagcctgcaggatcctttcttagtgactgtctgctccttgtatgtcaaatgaacaataacaccagaaaccccttagtcctatacccaggtaacaaccaaaaattcctntctggcaaaaggcagattgaaacagcttcttcctctttcagctctaatgtcactgtcttgtccccctgcgtcaagcatcagggctttctctgggtcagatggcacccattgggataaataaggtctaaagatgccgctggcagcttaggaaggggctggggagatgggagtgggatattgaaggaagatgaggataggggaggaaggaaagggggacagagggcatgaggtataaaggtaagacccccntagggcccccgctaaagcagcccccaggcacctgggccaccgtctcctgttcctcacccagaagatgccgcagatgtctcactgccagctgactgttggggttaaactcccattaggattcaaggatggttaccaccagccacggctgagccgttttccatcagatgtctctgtcccaccttagcaaggccatcatgactcatggactctgttttgtcaccagtaaacgccctgaggtt

SEQ ID NO: 21 - DNA, 1973 bp, Ovis sp.
cggccgctctagaactagtggatcccccgggctgcaggaattccaaaaatgccagcnattagatagcccgaacgtgtgtcattgtttctcccacacactcaagaccccaactgagaactaaggaccgaagtgactaagcgcggagcttccccacgtcagcctggctgaccactctccccgggcttccgcagctgcctctgcccttctcctcctagaggctttggcccccggagatctggccaaccagaacttcttcttccagaggttggatgggttcctgtttctgcttccatttccttgtttagaatgacatttctccccaaactgcaatcattcttacttaaaagtcggagctgtgataatagtttaaaatataacccattttgttcctttggtgatttttttttttctttatcaaagcgaaagcaaaggtttacttttcctattagcctcctccctgcaacccccctccacgggccttcccatctgtatcttttctgagcgccacatgggaagttgactgctcgcttgtacagagagtggtgaaggcccgggtggatctgagtgaggatctgtgcatcttcactcccaagaccgtccccagcccagagcccagaagccatcgccctcaccctactggtcaagtcctgatgtcaccacctgtgctgatcccaggggcatctgtgatggagaagggtgactctggaattttgacatttttactggaagaatctgtactgttatgcgtctggagcagatgcctagctcacctcatcctttggtatgagaggggatgagccagccaccctgtagactgatttactcctgatgttccacaaagaacagaatgtggcctgcggttccacgatggcttcaccctcgtattcgcagcttcacagaaccaaacagtaaattgtacaccctgagctctcctcttggaggccggaggggactgcttgtactctgcagcaataaatcctcccaagagtggatctggactttaggaggtgtgaagtttggtttgttttcaaataacagtgcccggagccaattgcaatgctttcatctctaaataatgttaaacctcacaagggccccctcccctccccctaggattgtttcccaagcttcagggcacaatagatttttttttcagagccatggcaaagtccctcttcatattgctcagatatattctactgtaaaccccggggtttttccagagaccaatattaaaaccacagtttatttttgtagaaatgaacaggaatccaaactgcaactaaggagcaaaaacaaatgccagttattattactcacaaaatgtcataactggccgtgagtgtgatcagtaaattaaacttagtccacatccgctgagcatgagtctgggggtgatgcaagttgtgttaagccaactcccgctttccttctctatcatccccacgctgtcacgtgataagtgtgtagtttggctcacaaaagacataaacatgttttcggaatgaataagtaaatagctaaaataccacatctattagaaggacctaagtccaaaacactgacaacacaaactgctggcaaaaatatggagcaagcagaactctgattcactgctggcgagaaggcaagatggttcagctgctttggaagaaagctgaacatattcttatcatacagtctagcaattgcatccttggtattcaccaaaaaaagctgaaactttacagccgcacagaaacttacatacagatgtttaaacgcagctttagtcacagtttctaaaacttggacataactaagatgtcctttagtaggtgaatggattaataaactgttgtggcacgtcaggtggtggaatatattatcagtgcaaaaacacaaatctgtcaagccatggaagagatagaggaacttaatgcatattattaagtaaagaaaccatctgaaagattgcctgctgtcagattcca

SEQ ID NO: 22 - DNA, 920 bp, Ovis sp.
agggaacaaaagctgggtacgatatcatacatagtgaaaaatacttgtcattacatatttgtcgaaacccatagaatacacacaaccaagagtgaactttttttagaaaaggaaatgagatagttaaaataagtttaaatcagagggtgtttgctctggtatttggatgggtcttataatggtggtagctggataatggagtggtgtttggggtaaccactagaaaattcttaaagggaatgtataatgtggaaggatattcatgtcccccataacccattctctgggtcccagagaagaagagagtggtgatgcttagtcttaaggcatccccaaaggacctaagaggcttgtcaccctttcatctgatagggcaggataacattggagcggatgttggggtttctgagagatgcctgagaactagcgccccctgatgttgatcacggggtacaaccttccagttataaaatgagcaagttctggggatctgatgtaaagcattgtggttattgttaataatattgttttctatactcataaagttattcagataataaatctcaaatcttctccccaaaacagaaatgatgattatgtgatgcaatgaaggtgttaactaatgctactgctgctgctgagtcgcttcagtcgtgtgcgactctgtgcgaccccatagacggcagcccaccaggctcccctgtccctgggattctccaggcaagaacactggagtgggttgccatttccttctccaatgcatgaaggtgaaaagagaaagtgaagtcactcagtcgtgtctgactcttagcgaccccatggactgcagcctaccagccttctccgtccatgggattttccaggcaagagtactggagtggggtgccattgccttctccgaactaatgctacagtggtgg

SEQ ID NO: 23 - DNA, 650 bp, Ovis sp.
cctcctccaggggctcttcctgaccccaggcgtcaaacccgcatctcctgcgtctcttgcatcacaggcagattctctacccactgagccatctgggaagcccttttataatatatgaatataccaaatcaacacttggcacattttaaacttaaactatgctatatgtcaattatatctcaataaaacaggaccaaaaaaggactagcacactttcacctacccggagtctgaaccaactgataaataagacaaagggataaataaggtagatggtagtgatgatcatcttcactgtgttaaaagctgatattgaagaatatgggttataaaactgaaggccctgggcctcctgatgttgaaccacagaattagaaagacgtaggcacccttcatccatagcatgagaaactgcaggtgtctggatgttggagaagggcctgtatcatttagctacattgcagggccctagatagatagagatctgtttaaacctcgagggccggagggtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaat

SEQ ID NO: 24 - DNA, 705 bp, Ovis sp.
tataatgtggttagaaataacgtctgactttgtgccagtacatttttgaatttgagagagtattattctggctacttcctcagaaaagtaacacttaattttaacttaaaaaaaaaaaaaaactaacaaaagaccaacacagcaagtacacattatttctccttgtaacattgagccttgtaatacgggagaatgaacctgtggtaatcggatgtaaattcccagtgatttcttacctattctcagttgcgggggaggggaatggatacactatcagttgaaccaaaaaagagaaaaaattatcatagacaaaagccagaaacattctacatgtgccaattaacttactggagagaagtacgcaaagggacgtgtttggcaacaagacaatgattgtgaggggctgtcctcttgatttcagtgtcttcctgtctctgctgggtctgaagcaacacagagttgcttttgcagcaggagaactcttagtaggacatagtctaccttggcgatcctcaaatggtttagtcagtgtcctttccttatcaggatattggcttctgtgccaagactagtcaagattcccagtacatatggtgaagatgttggggaatcacaggcaatctgagtcagtgggtgctcagcacaaaatgaaaacaaagcctccaagtaagttggccatgaaataagcaaagagagagtagaaag

SEQ ID NO: 25 - DNA, 900 bp, Ovis sp.
tactcacaagtgtgaaacaccatagaaggcatgaatacataataaatgatgcataccttttaaagagagaaagctagggtaagtggggatctactgttcaagtctcagggattcaaacccagcactgtctgactctaaatcctttgttctttccacagctcaagtctttctctttcaaacacacacacacacacacacacacacacacactttaaccattttgtttttgagaccgagggcatcaaggaatcaccaccaagaactctcaattcttgtactagcattaaagaaagagaacttagttgactcaaacttcacagatggatggtaggtctaaactcatcctggagcctggctggaaagcagtcaaacagaacctgagcgatacgaggctcagggaagttcaagggctgagagggtacagaggagagaaggtctgggaggtggaaacagtctcagaggggctgcttctgaattaagcactgagagaagagaaagggatcactaagaagtggaaatggaatgaggtttcggagagaggaacagcaggtacgaagcatggaggactattctgaggcttttagggagaccctggatgaaatctcataggagagcctgtgtgaaaagcagcaaagagcttgaaagggctttggctggttgggagctgcacatttgcccagcatctgtgtctttctttctcgctttatctccttcatagagtctatgtggtttccatggcaactaatgccatagttgggccaagtgacccagactaagctaatcaacattttccctccccccgcccccaggggggcagatgatctaaactagtccaatcaagttgaatctcaggcctttaattaagcggccgccaccgcggtggagctccattcgccctat

SEQ ID NO: 26 - DNA, 24 bp, Artificial Sequence
  Description of Artificial Sequence Probes and PCR Primers
gagaaaataatgaatgtcaaagga

SEQ ID NO: 27 - DNA, 20 bp, Artificial Sequence
  Description of Artificial Sequence Probes and PCR Primers
tgataatcccagcagtattc

SEQ ID NO: 28 - DNA, 22 bp, Artificial Sequence
  Description of Artificial Sequence Probes and PCR Primers
cttgatgggtttatccagaaca

SEQ ID NO: 29 - DNA, 23 bp, Artificial Sequence
  Description of Artificial Sequence Probes and PCR Primers
ctgtggatatattcccaaaacac

SEQ ID NO: 30 - DNA, 95 bp, Artificial Sequence; identical to SEQ ID NO: 14
  Description of Artificial Sequence Probes and PCR Primers

SEQ ID NO: 31 - DNA, 95 bp, Artificial Sequence
  Description of Artificial Sequence Probes and PCR Primers
aattcctgtggatatattcccaaaacacaacaatgacagttgagacaaccagcattgacagaatcacttttcctttgacattcattattttctcg

SEQ ID NO: 32 - DNA, 24 bp, Artificial Sequence
  Description of Artificial Sequence Probes and PCR Primers
ggtggtttccgagatggtttaaca

SEQ ID NO: 33 - DNA, 24 bp, Artificial Sequence
  Description of Artificial Sequence Probes and PCR Primers
gggttgaaccagtccgatagctta

SEQ ID NO: 34 - DNA, 21 bp, Artificial Sequence
  Description of Artificial Sequence Probes and PCR Primers
tccaggatgcctttgatagag

SEQ ID NO: 35 - DNA, 19 bp, Artificial Sequence
  Description of Artificial Sequence Probes and PCR Primers
gggaggaagcgaaggtgca

SEQ ID NO: 36 - DNA, 36 bp, Artificial Sequence
  Description of Artificial Sequence Probes and PCR Primers
acgtggctccaagaattctccaggcaagagtactgg

SEQ ID NO: 37 - DNA, 49 bp, Artificial Sequence
  Description of Artificial Sequence Probes and PCR Primers
catcttgttcaatggccgatcccattattttctcctgggaaaagaaaag

SEQ ID NO: 38 - DNA, 49 bp, Artificial Sequence
  Description of Artificial Sequence Probes and PCR Primers
cttttcttttcccaggagaaaataatgggatcggccattgaacaagatg

SEQ ID NO: 39 - DNA, 23 bp, Artificial Sequence
  Description of Artificial Sequence Probes and PCR Primers
caggtcgacggatccgaacaaac

SEQ ID NO: 40 - DNA, 47 bp, Artificial Sequence
  Description of Artificial Sequence Probes and PCR Primers
cagatctaacgaggattcaatgtcaaaggaaaagtgattctgtcaat

SEQ ID NO: 41 - DNA, 27 bp, Artificial Sequence
  Description of Artificial Sequence Probes and PCR Primers
ctgaactgaatgtttatccaggccatc

SEQ ID NO: 42 - DNA, 51 bp, Artificial Sequence
  Description of Artificial Sequence Probes and PCR Primers
gcgcaccgtgggcttgtactcggtcattattttctcctgggaaaagaaaag

SEQ ID NO: 43 - DNA, 34 bp, Artificial Sequence
  Description of Artificial Sequence Probes and PCR Primers
gagaaaataatgaccgagtacaagcccacggtgc

SEQ ID NO: 44 - DNA, 26 bp, Artificial Sequence
  Description of Artificial Sequence Probes and PCR Primers
ctggggatccagacatgataagatac

SEQ ID NO: 45 - DNA, 24 bp, Artificial Sequence
  Description of Artificial Sequence Probes and PCR Primers
ctggttggttctagaacaggagga

SEQ ID NO: 46 - DNA, 66 bp, Artificial Sequence
  Description of Artificial Sequence Probes and PCR Primers
catcttgttcaatggccgatcccattcctcctcctcctccactggtgacaaaacagagtccatgag

SEQ ID NO: 47 - DNA, 66 bp, Artificial Sequence
  Description of Artificial Sequence Probes and PCR Primers
ctcatggactctgttttgtcaccagtggaggaggaggaggaatgggatcggccattgaacaagatg

SEQ ID NO: 48 - DNA, 23 bp, Artificial Sequence
  Description of Artificial Sequence Probes and PCR Primers
caggtcgacggatccgaacaaac

SEQ ID NO: 49 - DNA, 22 bp, Artificial Sequence
  Description of Artificial Sequence Probes and PCR Primers
cagctgtgtgggtatgggaggg

SEQ ID NO: 50 - DNA, 27 bp, Artificial Sequence
  Description of Artificial Sequence Probes and PCR Primers
ctgaactgaatgtttatccaggccatc

SEQ ID NO: 51 - DNA, 27 bp, Artificial Sequence
  Description of Artificial Sequence Probes and PCR Primers
agccgattgtctgttgtgcccagtcat